(19) United States
(12) Patent Application Publication
(10) Pub. No.: US 2024/0295544 A1
(43) Pub. Date: Sep. 5, 2024

(54) ASSAY FOR SARS-CoV-2 INFECTION OF VULNERABLE HUMAN CELLS

(71) Applicant: The J. David Gladstone Institutes, a Testamentary Trust Established Under the Will of J. David Glads, San Francisco, CA (US)

(72) Inventors: Todd C. McDevitt, San Francisco, CA (US); Bruce Conklin, San Francisco, CA (US); Melanie Ott, Mill Valley, CA (US); Juan Perez-Bermejo, San Francisco, CA (US); Michael Sungwon Kang, San Francisco, CA (US); Sarah Rockwood, Kensington, CA (US); Camille Simoneau, San Francisco, CA (US); Gokul Ramadoss, Oakland, CA (US); David Joy, Oakland, CA (US)

(21) Appl. No.: 18/022,428

(22) PCT Filed: Aug. 24, 2021
(86) PCT No.: PCT/US21/47255

§ 371 Date: Feb. 21, 2023

Related U.S. Application Data
(60) Provisional application No. 63/069,361, filed on Aug. 24, 2020.

(51) Int. Cl. G01N 33/50; C12N 5/077; G01N 33/68

(52) U.S. Cl. CPC G01N 33/5061; C12N 5/0657; G01N 33/5035; G01N 33/6887; C12N 2503/02; C12N 2506/45; C12N 2510/00; G01N 2333/165

(57) ABSTRACT

Provided herein are methods and compositions useful for identifying compounds that can inhibit SARS-CoV-2 infection or the effects thereof, especially in cardiomyocytes (CMs), which are highly infectible by SARS-CoV-2 corona viruses.

### PRIORITY APPLICATION

This application is a U.S. National Stage Filing under 35 U.S.C. 371 from International Patent Application Serial No. PCT/US2021/047255, filed Aug. 24, 2021, published on Mar. 3, 2022 as WO2022/046706 which application claims benefit of priority to the filing date of U.S. Provisional Application Ser. No. 63/069,361, filed Aug. 24, 2020, the contents of which are specifically incorporated herein by reference in their entireties.

### GOVERNMENT SUPPORT

This invention was made with government support under ES032673 and RO1-HL135358 awarded by the National Institutes of Health, and under ERC 1648035 awarded by the National Science Foundation. The government has certain rights in the invention.

### INCORPORATION BY REFERENCE OF SEQUENCE LISTING PROVIDED AS A TEXT FILE

A Sequence Listing is provided herewith as a text file, “3730176US1.txt”, created on Feb. 14, 2024 and having a size of 446,507 bytes. The contents of the text file are incorporated by reference herein in their entirety.

### BACKGROUND

The World Health Organization has declared Covid-19 a global pandemic. A highly infectious coronavirus, officially called SARS-CoV-2, causes the Covid-19 disease. Even with the most effective containment strategies, the spread of the Covid-19 respiratory disease has only been slowed. The available vaccines are likely best way to prevent people from getting sick, but some refuse to be vaccinated and some vaccinated people can still suffer from Covid-19 infection. Compositions and methods to facilitate recovery from Covid-19 infection are needed.

### SUMMARY

Provided are methods and compositions useful for identifying compounds that can inhibit SARS-CoV-2 infection or the effects thereof. As illustrated herein, cardiomyocytes (CMs), are highly infectible by corona viruses, including SARS-CoV-2. Even low multiplicities of infection (MOI) of SARS-CoV-2 (e.g., about 1 virion particle per 1000 cells) can infect cardiomyocytes and support SARS-CoV-2 viral replication.

COVID-19 causes severe heart failure, but specific pathological consequences in cardiomyocytes have yet to be identified. Here the inventors describe the consequences of COVID-19 infection on cardiomyocytes, and upon the functioning of the heart. As demonstrated herein, human cardiomyocytes exposed to the virus exhibit significant myofibrillar disruption and a distinct patterns of sarcomeric fragmentation. Many cardiomyocytes exposed to coronavirus lack nuclear DNA by common detection methods, such as Hoechst or hematoxylin staining. In contrast, SARS-CoV-2 does not appear to infect induced pluripotent stem cells (iPSCs), endothelial cells, or cardiac fibroblasts. The adverse morphologic features of virally infected cardiomyocytes are distinct and potentially unique compared to other genetic or environmental stresses that are known to induce cardiomyopathy phenotypes.

Human iPSC-derived cardiac cells were used as described herein for infection with SARS-CoV-2 to reveal robust transcriptomic and morphological signatures in cardiomyocytes, which allowed identification of clear markers of viral damage in human autopsy specimens. Cardiomyocytes display a distinct pattern of sarcomere fragmentation, with specific cleavage of thick filaments, and COVID-19 autopsy samples displayed similar sarcomeric disruption. Numerous iPSC-cardiomyocytes lacked nuclear DNA. Surprisingly, enucleated cardiomyocytes were prevalent in the hearts of COVID-19 patients. These striking cytopathic features are useful for identifying new therapies for COVID-19-related heart failure.

Methods and assay mixtures are described herein that involve use of human cells, for example, cardiomyocytes or cells generated from human induced pluripotent stem cells (iPS) for identifying compounds useful for treatment of SARS-CoV-2. Screening of viral infection and cytopathic effects of such infection in cardiomyocytes can be performed in multi-well plate formats that are compatible with high-throughput screening platforms.

In some cases, cardiomyocytes derived from induced pluripotent stem cells of different genotypes are used in the assays, allowing identification of compounds for treatment of SARS-CoV-2 in patients with different genetically induced cardiac conditions.

The screening assay described herein provides multiple distinct visual indications of cytopathic effects induced by coronavirus that can be used to identify different cellular responses to coronavirus infection and to test whether compounds are useful therapeutics to attenuate adverse consequences of SARS-CoV-2 viral infection. The methods are highly sensitive and can provide information on multiple parameters useful for evaluating cytopathic effects of SARS-CoV-2 viral infection. Thus, in addition to serving as a frontline screening platform for prophylactic and therapeutic effects of the virus on cardiac cells, the methods also serve as a sensitive assay for distinct cytopathic effects that could adversely impact other human cells and tissues that are vulnerable to coronavirus infection and inflammatory responses.

The therapeutic target can, for example, be the titin protein at the M-line in relation to infection. Titin is involved in sarcomere assembly and function through its elastic adaptor and signaling domains. Titin's M-line region contains a unique kinase domain that may regulate sarcomere assembly via its substrate titin cap (T-cap). Studies indicate that the titin M-line region is required to form a continuous titin filament and to provide mechanical stability.

### DESCRIPTION OF THE FIGURES

FIG. 1A-1H illustrate that SARS-CoV-2 induces cytopathic effects in iPS-derived cardiac cell types, and productively infects cardiomyocytes. FIG. 1A graphically illustrates quantification of SARS-CoV-2 viral RNA by RT-qPCR quantification of the viral nucleocapsid (N5) gene in cell cultures exposed to SARS-CoV-2. Error bars: SEM. **: p-val<0.01, one-way ANOVE with Tukey's multiple comparisons. FIG. 1B graphically illustrates the toxicity of SARS-CoV-2 to cardiac cell types, as quantified by nuclear retention. Y-axis depicts the % of nuclei counted (relative to mock). Nuclei were counted automatically at 10× magnification (10 images/condition). Vehicle treatment (mock; left bars), heat inactivated SARS-CoV-2 (MOI=0.1; middle bars), and SARS-CoV-2 (MOI=0.006; right bars) nucleic counts are shown. FIG. 1C shows a representative image of a SARS-CoV-2 infected cardiomyocyte, as observed by transmission electron microscopy (TEM) of osmium tetroxide/potassium ferricyanide stained cells. Cells were exposed to SARS-CoV-2 virus for 48 hours at an MOI of 0.006 before fixation. This view shows the nucleus to the right, in addition to remnant ER-Golgi, with a closed membrane of viral particles. This image is a less magnified view of the images shown in FIG. 1D-1E; the line in the lower right corresponds to 0.5 μm. FIG. 1D shows an expanded view of the inset shown in FIG. 1C, further illustrating that SARS-CoV-2 virions were present and showing that a double membrane cannot be discerned by transmission electron microscopy. The line in the lower right corresponds to 0.4 μm. FIG. 1E shows an expanded view of the center of FIG. 1D, further illustrating that SARS-CoV-2 virions were present and showing the 500-750 nm diameter membrane and the 50-60 nm diameter viral particles within. The line in the lower right corresponds to 100 nm. FIG. 1F graphically illustrates ACE2 transcript levels in CMs compared to undifferentiated iPS cells as quantified by RT-qPCR quantification. **: p-value<0.01. FIG. 1G graphically illustrates the SARS-CoV-2 viral N5-fold change in infected iPSC and CM cells. Infection of iPSCs yielded no detectable levels of viral N5. FIG. 1H graphically illustrates the SARS-CoV-2 fold change as detected by viral N5 fold changes relative to the N5 levels in IPSCs.

FIG. 2A-2C illustrate pharmacological modulation of SARS-CoV-2 infection and host innate immune responses in CMs. FIG. 2A graphically illustrates viral Nucleocapsid (N5) levels of CM samples exposed to SARS-CoV-2 for 48 h (MOI=0.006) after 2 h pretreatment with the indicated reagents to block viral entry. RT-qPCR was used to quantify N5 levels. The agents used included a vehicle control (DMSO), an ACE2 blocking antibody (‘ACE2ab’), a PIKfyve inhibitor Apilimod, an autolysosome acidification blocker bafilomycin, a cathepsin-L inhibitor Z-Phe-Tyr(tBu)-diazomethylketone (Z-FY-DK), a serine protease inhibitor aprotinin, a cathepsin-B inhibitor CA-074, and a TMPRSS2 inhibitor camostat mesylate. Dots represent separate replicates. *: p-val<0.05, **: p-val<0.01. N>=3 for all conditions. One-way ANOVA with Tukey's multiple comparisons. FIG. 2B graphically illustrates SARS-CoV-2 RNA (N5) levels in CMs pretreated with different viral infection blocking agents as detected by RT-qPCR quantification of N5 levels. CMs were pretreated with either vehicle control (DMSO), ACE2 blocking antibody (‘ACE2ab’) or a cathepsin-B and -L blocker (E64D) for 2 hours before infection with SARS-CoV-2 (MOI=0.006). The graph depicts fold changes relative to a vehicle control (DMSO). Duplicates were analyzed for significance by one-way ANOVA with Tukey's multiple comparisons. ***: p-value<0.001. FIG. 2C graphically illustrates levels of factors that prime the cell's innate immune response in CM samples exposed to SARS-CoV-2 for 48 h (MOI=0.006) after 2 h pretreatment with the indicated reagents to block viral entry. Dots represent separate replicates. *: p-val<0.05, **: p-val<0.01. N>=3 for all conditions. One-way ANOVA with Tukey's multiple comparisons.

FIG. 3A-3J illustrate the transcriptional effects of SARS-CoV-2 exposure to cardiac cells. FIG. 3A graphically illustrates the percentage of total viral reads that map to the SARS-CoV-2 viral genome in multiple cell types. iPSCs, ECs or CFs were exposed at an MOI of 0.006, and CMs were exposed to the virus at three different MOIs: 0.001 (‘Low’), 0.01 (‘Mid’) and 0.1 (‘High’). **: p-val<0.01; ***: p-val<0.001. FIG. 3B graphically illustrates principal component analysis of transcriptomic samples. Dot shapes and colors represent the different cell types and whether they were exposed to SARS-CoV-2 virus and, in the case of CMs, the different MOIs used. FIG. 3C shows a loading plot for genes marking cardiomyocyte state (forward-slashed hatching ///), SARSCoV-2 infection related factors (no shading), and immune response (reverse-slashing \\\). FIG. 3D is a bar graph comparing genes involved in sarcomeric structure and myosin contractility between the high infection and mock infection CM groups. FIG. 3E graphically illustrates single cell transcript levels of ACE2 in iPS-derived cardiac cells. Each dot represents normalized transcript levels in a single cell. FIG. 3F graphically illustrates single cell transcript levels of FURIN in iPS-derived cardiac cells. Each dot represents normalized transcript levels in a single cell. FIG. 3G graphically illustrates single cell transcript levels of cathepsin-L (CTSL) in iPS-derived cardiac cells. Each dot represents normalized transcript levels in a single cell. FIG. 3H graphically illustrates single cell transcript levels of cathepsin-B (CTSB) in iPS-derived cardiac cells. Each dot represents normalized transcript levels in a single cell. FIG. 3I graphically illustrates single cell transcript levels of PIKfyve in iPS-derived cardiac cells. Each dot represents normalized transcript levels in a single cell. FIG. 3J graphically illustrates single cell transcript levels of DPP4 in iPS-derived cardiac cells. Each dot represents normalized transcript levels in a single cell.

FIG. 4A-4F illustrate cytopathological features induced by SARS-CoV-2 infection in CMs. FIG. 4A shows representative immunofluorescence images of myofibrillar fragmentation in CMs at different timepoints after exposure to SARS-CoV-2. White arrows indicate fragments consisting of two bands of cTnT positive staining. Scale bars: 50 μm. FIG. 4B graphically illustrates numbers of cells presenting myofibrillar fragmentation at 24 h and 48 h post-exposure to SARS-CoV-2 (defined as at least one event of a cTnT doublet unaligned and dissociated from other myofibrils). The number of cells was normalized to total number of nuclei in the images counted. Each dot represents a separate infection sample. Each replicate is the additive count of nine randomly acquired fields of view. ***: p-val<0.001. FIG. 4C shows representative images of immunostained cells infected with SARS-CoV-2, illustrating that cells staining positively for viral dsRNA are adjacent to other cells with different degrees of myofibrillar fragmentation. White squares indicate zoomed in areas, with labels corresponding to insets. White arrows point at examples of cTnT doublets (myofibrillar fragments). FIG. 4D shows representative images of stained CMs displaying myofibrillar fragmentation. White arrows indicate cTnT-ACTN2-cTnT staining positive fragments. Scale bars: 50 μm, inset view: 15 μm. FIG. 4E shows TEM images of sarcomeres in mock (‘healthy’) and SARS-CoV-2 infected (MOI=0.006) CM cultured cells (top). Darker gray arrows denote the sarcomeric z-disks; lighter gray arrows indicates M-line locations. Healthy sarcomeres display clear I and A-bands, but fragmented SARS-CoV-2 exposed sarcomeres only possess thin filaments. The image at the upper right is an expanded image of sarcomeric z-disks (arrows). The two images at the bottom are representative TEM image of a healthy nucleus (left), and a nucleus of a cell infected with SARS-CoV-2 (right). FIG. 4F shows an image of a cultured CM that was immunofluorescently stained after incubation with live SARS-CoV-2. The view to the right is an expanded view of the inset shown at the left, indicating that cells that have lost nuclear material.

FIG. 5A-5G illustrate pathological features of autopsy myocardial tissue from SARS-CoV-2 infected patients. FIG. 5A shows images of healthy neonatal left ventricle tissue stained with Hematoxylin and Eosin (H&E) to facilitate identification of the nucleus and other cellular structures. FIG. 5B shows images of H&E stained myocardial tissue from a COVID-19 patient with diagnosed myocarditis. Black boxes indicate the regions shown directly below that are at higher magnification. Arrows indicate cardiomyocytes showing a loss of nuclear material. FIG. 5C graphically illustrates the numbers of nuclei per field of view of intact myocardium and disrupted myocardium from SARS-CoV-2 patients. Statistical significance was determined by fitting to a Poisson generalized linear model, p-val<0.02. FIG. 5D shows representative H&E staining images of myocardial tissue from COVID-19 patients without diagnosed myocarditis. Darker gray arrows denote putative nuclear locations with loss of nuclear material. Lighter grey arrows indicate sarcomeric condensation. Black arrows indicate breakage at the intercalated disks between cardiomyocytes. FIG. 5E shows representative images from the myocardial tissue of a COVID-19 myocarditis patient immunohistochemical stained for troponin (cTnt, green in the original), collagen IV (grey in the original), and DAPI (blue in the original). Autofluorescence was also used to facilitate visualization of the images. Cardiomyocytes show diffuse and disorganized troponin staining with occasional cells in the blood vessel staining positively for troponin. White boxes indicate the regions shown directly below that are at higher magnification. White arrows indicate cardiac troponin T material in the cytoplasm of a mononuclear cell within a blood vessel. FIG. 5F shows images of a region of the heart from a COVID-19 patient denoting the transition from healthy to sick myocardium. White boxes indicate the regions shown to the right that are at higher magnification. The disrupted myocardium region is characterized by extensive breaks in α-actinin 2 (ACTN2) staining. FIG. 5G shows immunohistochemically stained images illustrating that viral nucleocapsid protein (magenta in the original; e.g., lower right-center) and α-actinin 2 (green in the original; striated tissue throughout) yielded no recognizable signal aside from occasional, unidentified puncta.

### DETAILED DESCRIPTION

As illustrated herein, cardiomyocytes (CMs) can easily be infected by corona viruses, including SARS-CoV-2. Methods are described herein for identifying compounds that can inhibit or prevent such infection.

Such methods can include (a) contacting cardiomyocytes with one or more test agents either before, during or after the cardiomyocytes have been contacted (infected) with corona viruses, for example SARS-CoV-2; and (b) observing whether the cardiomyocytes are enucleated, observing whether the cardiomyocytes have cleaved cardiac myofibrils, observing whether the cardiomyocytes have cleavages in their titin proteins. The assays can also include measuring the number or reproduction rate of the corona viruses compared to a control. The measurements can be performed at one or more time points after the cardiomyocytes are contacted with the one or more test agents. The control can be untreated cardiomyocytes, meaning cardiomyocytes that were not contacted with a test agent. In some cases, the control can be cardiomyocytes contacted with a compound or biological known to inhibit or prevent corona virus infection.

The cardiomyocytes can be obtained from a variety of sources, for example, from existing cardiomyocyte cell lines, from healthy subjects, and/or from patients with cardiac conditions or cardiac diseases. In some cases, the cardiomyocytes can be obtained from induced pluripotent stem cells (iPSCs), which can be generated from cells obtained from healthy subjects or from patients with cardiac conditions or cardiac diseases. For example, cardiomyocytes can be obtained from induced pluripotent stem cells (iPSCs) generated from cells with genetic mutations, including genetic mutations that adversely affect heart function, that adversely affect immune function, or a combination thereof. The cardiomyocytes can, in another example, be obtained from induced pluripotent stem cells (iPSCs) that have mutations in one or more of their immune-related genes, for example, in their innate immune genes. Such mutations can make an individual more vulnerable to COVID-19 infection.

### Test Agents

A variety of test agents (e.g., compounds and/or biological agents) can be tested to identify useful agent that reduce SARS-CoV-2 virally induced myofibrillar disruption, sarcomeric fragmentation, nuclear staining, enucleation, cardiac troponin solute levels, or a combination thereof in cardiomyocytes compared to a control assay of cardiomyocytes in the presence of SARS-CoV-2 virus without the test compound(s)/biological agents. For example, the test agents can be one or more small molecules, antibodies, nucleic acids, carbohydrates, proteins, peptides, or a combination thereof. Any such test agents can be tested and/or evaluated in the assays.

### Cells for Test Assays

A population of cardiomyocytes for testing can be derived from essentially any source and can be heterogeneous or homogeneous. In certain embodiments, the cells to be tested as described herein are adult cells, including adult cardiomyocytes from essentially any accessible source. In other embodiments, the cells used are cardiomyocytes generated from induced pluripotent stem cells (iPSCs). The cells used to generate the iPSCs can be adult cells, adult stem cells, progenitor cells, or somatic cells obtained from healthy subjects or from patients with cardiac conditions or cardiac diseases. In still other embodiments, the cells used to generate iPSCs include any type of cell from a newborn, including, but not limited to newborn cord blood, newborn stem cells, progenitor cells, and tissue-derived cells (e.g., somatic cells). Accordingly, a starting population of cells that is used to generate iPSCs, can be essentially any live somatic cell type.

The cardiomyocytes can be autologous or allogeneic cells (relative to a subject to be treated or who may receive the cells).

In some cases, cardiomyocytes from healthy subjects are used in the test assays. In other cases, cardiomyocytes from subjects with cardiac conditions are used in the test assays. Cardiomyocyte cell lines can be used in the test assays. Alternatively, the cardiomyocytes can be isolated from a healthy subject, a subject with a cardiac condition, or the cardiomyocytes can be generated from induced pluripotent stem cells (iPSCs) from either healthy subjects or subjects with a cardiac condition. For example, cardiomyocytes can be obtained from induced pluripotent stem cells (iPSCs) generated from cells with genetic mutations, including genetic mutations that adversely affect heart function, that adversely affect immune function, or a combination thereof. The cardiomyocytes can, in another example, be obtained from induced pluripotent stem cells (iPSCs) that have mutations in one or more of their immune-related genes, for example, in their innate immune genes. Such mutations can make an individual more vulnerable to COVID-19 infection.

Cardiomyocytes can be generated from induced pluripotent stem cells (iPSCs) by any convenient method. For example, the cardiomyocytes can be generated from iPSCs using the methods described in WO 2015/038704, which is incorporated herein by reference in its entirety.

Cardiomyocytes from subjects with a variety of cardiac diseases and conditions can be used in the assays described herein. For example, the cardiomyocytes can be from any subject with any cardiac pathology or cardiac dysfunction.

The terms “cardiac pathology” or “cardiac dysfunction” are used interchangeably and refer to any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathies), diseases such as angina pectoris, myocardial ischemia and/or infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart.

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder may be, for example, inflammatory, metabolic, toxic, infiltrative, fibroplastic, hematological, genetic, or unknown in origin. There are two general types of cardiomyopathies: ischemic (resulting from a lack of oxygen) and non-ischemic.

Ischemic cardiomyopathy is a chronic disorder caused by coronary artery disease (a disease in which there is atherosclerotic narrowing or occlusion of the coronary arteries on the surface of the heart). Coronary artery disease often leads to episodes of cardiac ischemia, in which the heart muscle is not supplied with enough oxygen-rich blood.

Non-ischemic cardiomyopathy is generally classified into three groups based primarily on clinical and pathological characteristics: dilated cardiomyopathy, hypertrophic cardiomyopathy and restrictive and infiltrative cardiomyopathy.

In another embodiment, the cardiac pathology is a genetic disease such as Duchenne muscular dystrophy and Emery Dreiffuss dilated cardiomyopathy.

For example, the cardiac pathology can be selected from the group consisting of congestive heart failure, myocardial infarction, cardiac ischemia, myocarditis and arrhythmia.

### Titin

Cardiac muscle is striated, like skeletal muscle, with actin and myosin arranged in sarcomeres to enable contractile function. The actin and myosin filaments have a specific and constant length of about a few micrometers. The filaments are organized into repeated subunits along the length of the myofibril. These subunits are called sarcomeres. Muscle cells are largely filled with myofibrils running parallel to each other along the long axis of the cell. The sarcomeric subunits of one myofibril are in nearly perfect alignment with those of the myofibrils next to it. This alignment provides optical properties so that cells to appear striped or striated.

Titin constitutes the third myofilament of cardiac muscle, with a single giant polypeptide spanning from Z-disk to the M-band region of the sarcomere. Titin has two general regions, an N-terminal I-band and a C-terminal A-band. An approximate 1.0 MDa region in the I-band is extensible and consists of tandemly arranged immunoglobulin (Ig)-like domains that make up proximal (near Z-disk) and distal (near A-I junction) segments, interspersed by the PEVK sequence (rich in proline, glutamate, valine, and lysine residues) and an N2B element.

The C-terminal titin region of about 2 MDa includes the A-band and is inextensible. This C-terminal region is composed of regular arrays of Ig and fibronectin type 3 (Fn3) modules forming so-called super-repeats. The A-band is thought to act as a protein-ruler and possesses kinase activity. An N-terminal Z-disc region and a C-terminal M-band region bind to the Z-line and M-line of the sarcomere, respectively, so that a single titin molecule spans half the length of a sarcomere. Titin also contains binding sites for muscle associated proteins and serves as an adhesion template for assembly of contractile machinery in muscle cells. The M-band is encoded by TTN exons 359-364.

Considerable variability exists in the I-band, the M-line, and the Z-disc regions of titin. Variability in the I-band region contributes to the differences in elasticity of different titin isoforms and, therefore, to the differences in elasticity of different muscle types. Mutations in this gene are associated with familial hypertrophic cardiomyopathy. Autoantibodies to titin are produced in patients with the autoimmune disease scleroderma.

The titin protein is encoded by the TTN gene, which is located on human chromosome 2, at NC_000002.12 (178525989..178807423, complement; see website at ncbi.nlm.nih.gov/gene?LinkName=protein_gene&from_uid=291045223). Alternative splicing of the TTN gene results in multiple transcript variants.

One example of a human titin protein sequence has UniProt accession number A0A0A0MRA3-1; this titin protein sequence is shown below as SEQ ID NO:1.

```
                10 20 30 40 50
MTTQAPTFTQ PLQSVVVLEG STATFEAHIS GFPVPEVSWF RDGQVISTST
                60 70 80 90 100
LPGVQISFSD GRAKLTIPAV TKANSGRYSL KATNGSGQAT STAELLVKAE
              110 120 130 140 150
TAPPNFVQRL QSMTVRQGSQ VRLQVRVTGI PTPVVKFYRD GAEIQSSLDF
              160 170 180 190 200
QISQEGDLYS LLIAEAYPED SGTYSVNATN SVGRATSTAE LLVQGEEEVP
              210 220 230 240 250
AKKTKTIVST AQISESRQTR IEKKIEAHED ARSIATVEMV IDGAAGQQLP
              260 270 280 290 300
HKTPPRIPPK PKSRSPTPPS IAAKAQLARQ QSPSPIRHSP SPVRHVRAPT
              310 320 330 340 350
PSPVRSVSPA ARISTSPIRS VRSPLLMRKT QASTVATGPE VPPPWKQEGY
              360 370 380 390 400
VASSSEAEMR ETTLTTSTQI RTEERWEGRY GVQEQVTISG AAGAAASVSA
              410 420 430 440 450
SASYAAEAVA TGAKEVKQDA DKSAAVATVV AAVDMARVRE PVISAVEQTA
              460 470 480 490 500
QRTTTTAVHI QPAQEQVRKE AEKTAVTKVV VAADKAKEQE LKSRTKEVIT
              510 520 530 540 550
TKQEQMHVTH EQIRKETEKT FVPKVVISAA KAKEQETRIS EEITKKQKQV
              560 570 580 590 600
TQEAIMKETR KTVVPKVIVA TPKVKEQDLV SRGREGITTK REQVQITQEK
              610 620 630 640 650
MRKEAEKTAL STIAVATAKA KEQETILRTR ETMATRQEQI QVTHGKVDVG
              660 670 680 690 700
KKAEAVATVV AAVDQARVRE PREPGHLEES YAQQTTLEYG YKERISAAKV
              710 720 730 740 750
AEPPQRPASE PHVVPKAVKP RVIQAPSETH IKTTDQKGMH ISSQIKKTTD
              760 770 780 790 800
LTTERLVHVD KRPRTASPHF TVSKISVPKT EHGYEASIAG SAIATLQKEL
              810 820 830 840 850
SATSSAQKIT KSVKAPTVKP SETRVRAEPT PLPQFPFADT PDTYKSEAGV
              860 870 880 890 900
EVKKEVGVSI TGTTVREERF EVLHGREAKV TETARVPAPV EIPVTPPTLV
              910 920 930 940 950
SGLKNVTVIE GESVTLECHI SGYPSPTVTW YREDYQIESS IDFQITFQSG
              960 970 980 990 1000
IARLMIREAF AEDSGRFTCS AVNEAGTVST SCYLAVQVSE EFEKETTAVT
            1010 1020 1030 1040 1050
EKFTTEEKRF VESRDVVMTD TSLTEEQAGP GEPAAPYFIT KPVVQKLVEG
            1060 1070 1080 1090 1100
GSVVFGCQVG GNPKPHVYWK KSGVPLTTGY RYKVSYNKQT GECKLVISMT
            1110 1120 1130 1140 1150
FADDAGEYTI VVRNKHGETS ASASLLEEAD YELLMKSQQE MLYQTQVTAF
            1160 1170 1180 1190 1200
VQEPKVGETA PGFVYSEYEK EYEKEQALIR KKMAKDTVVV RTYVEDQEFH
            1210 1220 1230 1240 1250
ISSFEERLIK EIEYRIIKTT LEELLEEDGE EKMAVDISES EAVESGEDSR
            1260 1270 1280 1290 1300
IKNYRILEGM GVTFHCKMSG YPLPKIAWYK DGKRIKHGER YQMDFLQDGR
            1310 1320 1330 1340 1350
ASLRIPVVLP EDEGIYTAFA SNIKGNAICS GKLYVEPAAP LGAPTYIPTL
            1360 1370 1380 1390 1400
EPVSRIRSLS PRSVSRSPIR MSPARMSPAR MSPARMSPAR MSPGRRLEET
            1410 1420 1430 1440 1450
DESQLERLYK PVFVLKPVSF KCLEGQTARF DLKVVGRPMP ETFWFHDGQQ
            1460 1470 1480 1490 1500
IVNDYTHKVV IKEDGTQSLI IVPATPSDSG EWTVVAQNRA GRSSISVILT
            1510 1520 1530 1540 1550
VEAVEHQVKP MFVEKLKNVN IKEGSRLEMK VRATGNPNPD IVWLKNSDII
            1560 1570 1580 1590 1600
VPHKYPKIRI EGTKGEAALK IDSTVSQDSA WYTATAINKA GRDTTRCKVN
            1610 1620 1630 1640 1650
VEVEFAEPEP ERKLIIPRGT YRAKEIAAPE LEPLHLRYGQ EQWEEGDLYD
            1660 1670 1680 1690 1700
KEKQQKPFFK KKLTSLRLKR FGPAHFECRL TPIGDPTMVV EWLHDGKPLE
            1710 1720 1730 1740 1750
AANRLRMINE FGYCSLDYGV AYSRDSGIIT CRATNKYGTD HTSATLIVKD
            1760 1770 1780 1790 1800
EKSLVEESQL PEGRKGLQRI EELERMAHEG ALTGVTTDQK EKQKPDIVLY
            1810 1820 1830 1840 1850
PEPVRVLEGE TARFRCRVTG YPQPKVNWYL NGQLIRKSKR FRVRYDGIHY
            1860 1870 1880 1890 1900
LDIVDCKSYD TGEVKVTAEN PEGVIEHKVK LEIQQREDFR SVLRRAPEPR
            1910 1920 1930 1940 1950
PEFHVHEPGK LQFEVQKVDR PVDTTETKEV VKLKRAERIT HEKVPEESEE
            1960 1970 1980 1990 2000
LRSKFKRRTE EGYYEAITAV ELKSRKKDES YEELLRKTKD ELLHWTKELT
            2010 2020 2030 2040 2050
EEEKKALAEE GKITIPTFKP DKIELSPSME APKIFERIQS QTVGQGSDAH
            2060 2070 2080 2090 2100
FRVRVVGKPD PECEWYKNGV KIERSDRIYW YWPEDNVCEL VIRDVTAEDS
            2110 2120 2130 2140 2150
ASIMVKAINI AGETSSHAFL LVQAKQLITF TQELQDVVAK EKDTMATFEC
            2160 2170 2180 2190 2200
ETSEPFVKVK WYKDGMEVHE GDKYRMHSDR KVHFLSILTI DTSDAEDYSC
            2210 2220 2230 2240 2250
VLVEDENVKT TAKLIVEGAV VEFVKELQDI EVPESYSGEL ECIVSPENIE
            2260 2270 2280 2290 2300
GKWYHNDVEL KSNGKYTITS RRGRQNLTVK DVTKEDQGEY SFVIDGKKTT
            2310 2320 2330 2340 2350
CKLKMKPRPI AILQGLSDQK VCEGDIVQLE VKVSLESVEG VWMKDGQEVQ
            2360 2370 2380 2390 2400
PSDRVHIVID KQSHMLLIED MTKEDAGNYS FTIPALGLST SGRVSVYSVD
            2410 2420 2430 2440 2450
VITPLKDVNV IEGTKAVLEC KVSVPDVTSV KWYLNDEQIK PDDRVQAIVK
            2460 2470 2480 2490 2500
GTKQRLVINR THASDEGPYK LIVGRVETNC NLSVEKIKII RGLRDLTCTE
            2510 2520 2530 2540 2550
TQNVVFEVEL SHSGIDVLWN FKDKEIKPSS KYKIEAHGKI YKLTVLNMMK
            2560 2570 2580 2590 2600
DDEGKYTFYA GENMTSGKLT VAGGAISKPL TDQTVAESQE AVFECEVANP
            2610 2620 2630 2640 2650
DSKGEWLRDG KHLPLTNNIR SESDGHKRRL IIAATKLDDI GEYTYKVATS
            2660 2670 2680 2690 2700
KTSAKLKVEA VKIKKTLKNL TVTETQDAVF TVELTHPNVK GVQWIKNGVV
            2710 2720 2730 2740 2750
LESNEKYAIS VKGTIYSLRI KNCAIVDESV YGFRLGRLGA SARLHVETVK
            2760 2770 2780 2790 2800
IIKKPKDVTA LENATVAFEV SVSHDTVPVK WFHKSVEIKP SDKHRLVSER
            2810 2820 2830 2840 2850
KVHKLMLQNI SPSDAGEYTA VVGQLECKAK LFVETLHITK TMKNIEVPET
            2860 2870 2880 2890 2900
KTASFECEVS HFNVPSMWLK NGVEIEMSEK FKIVVQGKLH QLIIMNTSTE
            2910 2920 2930 2940 2950
DSAEYTFVCG NDQVSATLTV TPIMITSMLK DINAEEKDTI TFEVTVNYEG
            2960 2970 2980 2990 3000
ISYKWLKNGV EIKSTDKCQM RTKKLTHSLN IRNVHFGDAA DYTFVAGKAT
            3010 3020 3030 3040 3050
STATLYVEAR HIEFRKHIKD IKVLEKKRAM FECEVSEPDI TVQWMKDDQE
            3060 3070 3080 3090 3100
LQITDRIKIQ KEKYVHRLLI PSTRMSDAGK YTVVAGGNVS TAKLFVEGRD
            3110 3120 3130 3140 3150
VRIRSIKKEV QVIEKQRAVV EFEVNEDDVD AHWYKDGIEI NFQVQERHKY
            3160 3170 3180 3190 3200
VVERRIHRMF ISETRQSDAG EYTFVAGRNR SSVTLYVNAP EPPQVLQELQ
            3210 3220 3230 3240 3250
PVTVQSGKPA RFCAVISGRP QPKISWYKEE QLLSTGFKCK FLHDGQEYTL
            3260 3270 3280 3290 3300
LLIEAFPEDA AVYTCEAKND YGVATTSASL SVEVPEVVSP DQEMPVYPPA
            3310 3320 3330 3340 3350
IITPLQDTVT SEGQPARFQC RVSGTDLKVS WYSKDKKIKP SRFFRMTQFE
            3360 3370 3380 3390 3400
DTYQLEIAEA YPEDEGTYTF VASNAVGQVS STANLSLEVQ ALDRQSSGKD
            3410 3420 3430 3440 3450
VRESTKSQAV ADSSFTKEES KISQKEIKSF QGSSYEYEVQ VFESVSQSSI
            3460 3470 3480 3490 3500
HTAASVQDTQ LCHTASLSQI AESTELSKEC AKESTGEAPK IFLHLQDVTV
            3510 3520 3530 3540 3550
KCGDTAQFLC VLKDDSFIDV TWTHEGAKIE ESERLKQSQN GNIQFLTICN
            3560 3570 3580 3590 3600
VQLVDQGLYS CIVHNDCGER TTSAVLSVEG APESILHERI EQEIEMEMKE
            3610 3620 3630 3640 3650
FSSSFLSAEE EGLHSAELQL SKINETLELL SESPVYPTKF DSEKEGTGPI
            3660 3670 3680 3690 3700
FIKEVSNADI SMGDVATLSV TVIGIPKPKI QWFFNGVLLT PSADYKFVFD
            3710 3720 3730 3740 3750
GDDHSLIILF TKLEDEGEYT CMASNDYGKT ICSAYLKINS KGEGHKDTET
            3760 3770 3780 3790 3800
ESAVAKSLEK LGGPCPPHFL KELKPIRCAQ GLPAIFEYTV VGEPAPTVTW
            3810 3820 3830 3840 3850
FKENKQLCTS VYYTIIHNPN GSGTFIVNDP QREDSGLYIC KAENMLGEST
            3860 3870 3880 3890 3900
CAAELLVLLE DTDMTDTPCK AKSTPEAPED FPQTPLKGPA VEALDSEQEI
            3910 3920 3930 3940 3950
ATFVKDTILK AALITEENQQ LSYEHIAKAN ELSSQLPLGA QELQSILEQD
            3960 3970 3980 3990 4000
KLTPESTREF LCINGSIHFQ PLKEPSPNLQ LQIVQSQKTF SKEGILMPEE
            4010 4020 4030 4040 4050
PETQAVLSDT EKIFPSAMSI EQINSLTVEP LKTLLAEPEG NYPQSSIEPP
            4060 4070 4080 4090 4100
MHSYLTSVAE EVLSPKEKTV SDTNREQRVT LQKQEAQSAL ILSQSLAEGH
            4110 4120 4130 4140 4150
VESLQSPDVM ISQVNYEPLV PSEHSCTEGG KILIESANPL ENAGQDSAVR
            4160 4170 4180 4190 4200
IEEGKSLRFP LALEEKQVLL KEEHSDNVVM PPDQIIESKR EPVAIKKVQE
            4210 4220 4230 4240 4250
VQGRDLLSKE SLLSGIPEEQ RLNLKIQICR ALQAAVASEQ PGLFSEWLRN
            4260 4270 4280 4290 4300
IEKVEVEAVN ITQEPRHIMC MYLVTSAKSV TEEVTIIIED VDPQMANLKM
            4310 4320 4330 4340 4350
ELRDALCAII YEEIDILTAE GPRIQQGAKT SLQEEMDSFS GSQKVEPITE
            4360 4370 4380 4390 4400
PEVESKYLIS TEEVSYFNVQ SRVKYLDATP VTKGVASAVV SDEKQDESLK
            4410 4420 4430 4440 4450
PSEEKEESSS ESGTEEVATV KIQEAEGGLI KEDGPMIHTP LVDTVSEEGD
            4460 4470 4480 4490 4500
IVHLTTSITN AKEVNWYFEN KLVPSDEKFK CLQDQNTYTL VIDKVNTEDH
            4510 4520 4530 4540 4550
QGEYVCEALN DSGKTATSAK LTVVKRAAPV IKRKIEPLEV ALGHLAKFTC
            4560 4570 4580 4590 4600
EIQSAPNVRF QWFKAGREIY ESDKCSIRSS KYISSLEILR TQVVDCGEYT
            4610 4620 4630 4640 4650
CKASNEYGSV SCTATLTVTV PGGEKKVRKL LPERKPEPKE EVVLKSVLRK
            4660 4670 4680 4690 4700
RPEEEEPKVE PKKLEKVKKP AVPEPPPPKP VEEVEVPTVT KRERKIPEPT
            4710 4720 4730 4740 4750
KVPEIKPAIP LPAPEPKPKP EAEVKTIKPP PVEPEPTPIA APVTVPVVGK
            4760 4770 4780 4790 4800
KAEAKAPKEE AAKPKGPIKG VPKKTPSPIE AERRKLRPGS GGEKPPDEAP
            4810 4820 4830 4840 4850
FTYQLKAVPL KFVKEIKDII LTESEFVGSS AIFECLVSPS TAITTWMKDG
            4860 4870 4880 4890 4900
SNIRESPKHR FIADGKDRKL HIIDVQLSDA GEYTCVLRLG NKEKTSTAKL
            4910 4920 4930 4940 4950
VVEELPVRFV KTLEEEVTVV KGQPLYLSCE LNKERDVVWR KDGKIVVEKP
            4960 4970 4980 4990 5000
GRIVPGVIGL MRALTINDAD DTDAGTYTVT VENANNLECS SCVKVVEVIR
            5010 5020 5030 5040 5050
DWLVKPIRDQ HVKPKGTAIF ACDIAKDTPN IKWFKGYDEI PAEPNDKTEI
            5060 5070 5080 5090 5100
LRDGNHLYLK IKNAMPEDIA EYAVEIEGKR YPAKLTLGER EVELLKPIED
            5110 5120 5130 5140 5150
VTIYEKESAS FDAEISEADI PGQWKLKGEL LRPSPTCEIK AEGGKRFLTL
            5160 5170 5180 5190 5200
HKVKLDQAGE VLYQALNAIT TAILTVKEIE LDFAVPLKDV TVPERRQARF
            5210 5220 5230 5240 5250
ECVLTREANV IWSKGPDIIK SSDKFDIIAD GKKHILVIND SQFDDEGVYT
            5260 5270 5280 5290 5300
AEVEGKKTSA RLFVTGIRLK FMSPLEDQTV KEGETATFVC ELSHEKMHVV
            5310 5320 5330 5340 5350
WFKNDAKLHT SRTVLISSEG KTHKLEMKEV TLDDISQIKA QVKELSSTAQ
            5360 5370 5380 5390 5400
LKVLEADPYF TVKLHDKTAV EKDEITLKCE VSKDVPVKWF KDGEEIVPSP
            5410 5420 5430 5440 5450
KYSIKADGLR RILKIKKADL KDKGEYVCDC GTDKTKANVT VEARLIKVEK
            5460 5470 5480 5490 5500
PLYGVEVFVG ETAHFEIELS EPDVHGQWKL KGQPLTASPD CEIIEDGKKH
            5510 5520 5530 5540 5550
ILILHNCQLG MTGEVSFQAA NAKSAANLKV KELPLIFITP LSDVKVFEKD
            5560 5570 5580 5590 5600
EAKFECEVSR EPKTFRWLKG TQEITGDDRF ELIKDGTKHS MVIKSAAFED
            5610 5620 5630 5640 5650
EAKYMFEAED KHTSGKLIIE GIRLKFLTPL KDVTAKEKES AVFTVELSHD
            5660 5670 5680 5690 5700
NIRVKWFKND QRLHTTRSVS MQDEGKTHSI TFKDLSIDDT SQIRVEAMGM
            5710 5720 5730 5740 5750
SSEAKLTVLE GDPYFTGKLQ DYTGVEKDEV ILQCEISKAD APVKWFKDGK
            5760 5770 5780 5790 5800
EIKPSKNAVI KADGKKRMLI LKKALKSDIG QYTCDCGTDK TSGKLDIEDR
            5810 5820 5830 5840 5850
EIKLVRPLHS VEVMETETAR FETEISEDDI HANWKLKGEA LLQTPDCEIK
            5860 5870 5880 5890 5900
EEGKIHSLVL HNCRLDQTGG VDFQAANVKS SAHLRVKPRV IGLLRPLKDV
            5910 5920 5930 5940 5950
TVTAGETATF DCELSYEDIP VEWYLKGKKL EPSDKVVPRS EGKVHTLTLR
            5960 5970 5980 5990 6000
DVKLEDAGEV QLTAKDFKTH ANLFVKEPPV EFTKPLEDQT VEEGATAVLE
            6010 6020 6030 6040 6050
CEVSRENAKV KWFKNGTEIL KSKKYEIVAD GRVRKLVIHD CTPEDIKTYT
            6060 6070 6080 6090 6100
CDAKDFKTSC NLNVVPPHVE FLRPLTDLQV REKEMARFEC ELSRENAKVK
            6110 6120 6130 6140 6150
WFKDGAEIKK GKKYDIISKG AVRILVINKC LLDDEAEYSC EVRTARTSGM
            6160 6170 6180 6190 6200
LTVLEEEAVF TKNLANIEVS ETDTIKLVCE VSKPGAEVIW YKGDEEIIET
            6210 6220 6230 6240 6250
GRYEILTEGR KRILVIQNAH LEDAGNYNCR LPSSRTDGKV KVHELAAEFI
            6260 6270 6280 6290 6300
SKPQNLEILE GEKAEFVCSI SKESFPVQWK RDDKTLESGD KYDVIADGKK
            6310 6320 6330 6340 6350
RVLVVKDATL QDMGTYVVMV GAARAAAHLT VIEKLRIVVP LKDTRVKEQQ
            6360 6370 6380 6390 6400
EVVENCEVNT EGAKAKWFRN EEAIFDSSKY IILQKDLVYT LRIRDAHLDD
            6410 6420 6430 6440 6450
QANYNVSLTN HRGENVKSAA NLIVEEEDLR IVEPLKDIET MEKKSVTFWC
            6460 6470 6480 6490 6500
KVNRLNVTLK WTKNGEEVPF DNRVSYRVDK YKHMLTIKDC GFPDEGEYIV
            6510 6520 6530 6540 6550
TAGQDKSVAE LLIIEAPTEF VEHLEDQTVT EFDDAVFSCQ LSREKANVKW
            6560 6570 6580 6590 6600
YRNGREIKEG KKYKFEKDGS IHRLIIKDCR LDDECEYACG VEDRKSRARL
            6610 6620 6630 6640 6650
FVEEIPVEII RPPQDILEAP GADVVFLAEL NKDKVEVQWL RNNMVVVQGD
            6660 6670 6680 6690 6700
KHQMMSEGKI HRLQICDIKP RDQGEYRFIA KDKEARAKLE LAAAPKIKTA
            6710 6720 6730 6740 6750
DQDLVVDVGK PLTMVVPYDA YPKAEAEWFK ENEPLSTKTI DTTAEQTSFR
            6760 6770 6780 6790 6800
ILEAKKGDKG RYKIVLQNKH GKAEGFINLK VIDVPGPVRN LEVTETEDGE
            6810 6820 6830 6840 6850
VSLAWEEPLT DGGSKIIGYV VERRDIKRKT WVLATDRAES CEFTVTGLQK
            6860 6870 6880 6890 6900
GGVEYLFRVS ARNRVGTGEP VETDNPVEAR SKYDVPGPPL NVTITDVNRF
            6910 6920 6930 6940 6950
GVSLTWEPPE YDGGAEITNY VIELRDKTSI RWDTAMTVRA EDLSATVTDV
            6960 6970 6980 6990 7000
VEGQEYSFRV RAQNRIGVGK PSAATPFVKV ADPIERPSPP VNLTSSDQTQ
            7010 7020 7030 7040 7050
SSVQLKWEPP LKDGGSPILG YIIERCEEGK DNWIRCNMKL VPELTYKVTG
            7060 7070 7080 7090 7100
LEKGNKYLYR VSAENKAGVS DPSEILGPLT ADDAFVEPTM DLSAFKDGLE
            7110 7120 7130 7140 7150
VIVPNPITIL VPSTGYPRPT ATWCFGDKVL ETGDRVKMKT LSAYAELVIS
            7160 7170 7180 7190 7200
PSERSDKGIY TLKLENRVKT ISGEIDVNVI ARPSAPKELK FGDITKDSVH
            7210 7220 7230 7240 7250
LTWEPPDDDG GSPLTGYVVE KREVSRKTWT KVMDFVTDLE FTVPDLVQGK
            7260 7270 7280 7290 7300
EYLFKVCARN KCGPGEPAYV DEPVNMSTPA TVPDPPENVK WRDRTANSIF
            7310 7320 7330 7340 7350
LTWDPPKNDG GSRIKGYIVE RCPRGSDKWV ACGEPVAETK MEVTGLEEGK
            7360 7370 7380 7390 7400
WYAYRVKALN RQGASKPSRP TEEIQAVDTQ EAPEIFLDVK LLAGLTVKAG
            7410 7420 7430 7440 7450
TKIELPATVT GKPEPKITWT KADMILKQDK RITIENVPKK STVTIVDSKR
            7460 7470 7480 7490 7500
SDTGTYIIEA VNVCGRATAV VEVNVLDKPG PPAAFDITDV TNESCLLTWN
            7510 7520 7530 7540 7550
PPRDDGGSKI TNYVVERRAT DSEVWHKLSS TVKDTNFKAT KLIPNKEYIF
            7560 7570 7580 7590 7600
RVAAENMYGV GEPVQASPIT AKYQFDPPGP PTRLEPSDIT KDAVTLTWCE
            7610 7620 7630 7640 7650
PDDDGGSPIT GYWVERLDPD TDKWVRCNKM PVKDTTYRVK GLTNKKKYRF
            7660 7670 7680 7690 7700
RVLAENLAGP GKPSKSTEPI LIKDPIDPPW PPGKPTVKDV GKTSVRLNWT
            7710 7720 7730 7740 7750
KPEHDGGAKI ESYVIEMLKT GTDEWVRVAE GVPTTQHLLP GLMEGQEYSF
            7760 7770 7780 7790 7800
RVRAVNKAGE SEPSEPSDPV LCREKLYPPS PPRWLEVINI TKNTADLKWT
            7810 7820 7830 7840 7850
VPEKDGGSPI TNYIVEKRDV RRKGWQTVDT TVKDTKCTVT PLTEGSLYVF
            7860 7870 7880 7890 7900
RVAAENAIGQ SDYTEIEDSV LAKDTFTTPG PPYALAVVDV TKRHVDLKWE
            7910 7920 7930 7940 7950
PPKNDGGRPI QRYVIEKKER LGTRWVKAGK TAGPDCNFRV TDVIEGTEVQ
            7960 7970 7980 7990 8000
FQVRAENEAG VGHPSEPTEI LSIEDPTSPP SPPLDLHVTD AGRKHIAIAW
            8010 8020 8030 8040 8050
KPPEKNGGSP IIGYHVEMCP VGTEKWMRVN SRPIKDLKFK VEEGVVPDKE
            8060 8070 8080 8090 8100
YVLRVRAVNA IGVSEPSEIS ENVVAKDPDC KPTIDLETHD IIVIEGEKLS
            8110 8120 8130 8140 8150
IPVPFRAVPV PTVSWHKDGK EVKASDRLTM KNDHISAHLE VPKSVRADAG
            8160 8170 8180 8190 8200
IYTITLENKL GSATASINVK VIGLPGPCKD IKASDITKSS CKLTWEPPEF
            8210 8220 8230 8240 8250
DGGTPILHYV LERREAGRRT YIPVMSGENK LSWTVKDLIP NGEYFFRVKA
            8260 8270 8280 8290 8300
VNKVGGGEYI ELKNPVIAQD PKQPPDPPVD VEVHNPTAEA MTITWKPPLY
            8310 8320 8330 8340 8350
DGGSKIMGYI IEKIAKGEER WKRCNEHLVP ILTYTAKGLE EGKEYQFRVR
            8360 8370 8380 8390 8400
AENAAGISEP SRATPPTKAV DPIDAPKVIL RTSLEVKRGD EIALDASISG
            8410 8420 8430 8440 8450
SPYPTITWIK DENVIVPEEI KKRAAPLVRR RKGEVQEEEP FVLPLTQRLS
            8460 8470 8480 8490 8500
IDNSKKGESQ LRVRDSLRPD HGLYMIKVEN DHGIAKAPCT VSVLDTPGPP
            8510 8520 8530 8540 8550
INFVFEDIRK TSVLCKWEPP LDDGGSEIIN YTLEKKDKTK PDSEWIVVTS
            8560 8570 8580 8590 8600
TLRHCKYSVT KLIEGKEYLF RVRAENRFGP GPPCVSKPLV AKDPFGPPDA
            8610 8620 8630 8640 8650
PDKPIVEDVT SNSMLVKWNE PKDNGSPILG YWLEKREVNS THWSRVNKSL
            8660 8670 8680 8690 8700
LNALKANVDG LLEGLTYVFR VCAENAAGPG KFSPPSDPKT AHDPISPPGP
            8710 8720 8730 8740 8750
PIPRVTDTSS TTIELEWEPP AFNGGGEIVG YFVDKQLVGT NEWSRCTEKM
            8760 8770 8780 8790 8800
IKVRQYTVKE IREGADYKLR VSAVNAAGEG PPGETQPVTV AEPQEPPAVE
            8810 8820 8830 8840 8850
LDVSVKGGIQ IMAGKTLRIP AVVTGRPVPT KVWTKEEGEL DKDRVVIDNV
            8860 8870 8880 8890 8900
GTKSELIIKD ALRKDHGRYV ITATNSCGSK FAAARVEVFD VPGPVLDLKP
            8910 8920 8930 8940 8950
VVTNRKMCLL NWSDPEDDGG SEITGFIIER KDAKMHTWRQ PIETERSKCD
            8960 8970 8980 8990 9000
ITGLLEGQEY KFRVIAKNKF GCGPPVEIGP ILAVDPLGPP TSPERLTYTE
            9010 9020 9030 9040 9050
RTKSTITLDW KEPRSNGGSP IQGYIIEKRR HDKPDFERVN KRLCPTTSFL
            9060 9070 9080 9090 9100
VENLDEHQMY EFRVKAVNEI GESEPSLPLN VVIQDDEVPP TIKLRLSVRG
            9110 9120 9130 9140 9150
DTIKVKAGEP VHIPADVTGL PMPKIEWSKN ETVIEKPTDA LQITKEEVSR
            9160 9170 9180 9190 9200
SEAKTELSIP KAVREDKGTY TVTASNRLGS VFRNVHVEVY DRPSPPRNLA
            9210 9220 9230 9240 9250
VTDIKAESCY LTWDAPLDNG GSEITHYVID KRDASRKKAE WEEVTNTAVE
            9260 9270 9280 9290 9300
KRYGIWKLIP NGQYEFRVRA VNKYGISDEC KSDKVVIQDP YRLPGPPGKP
            9310 9320 9330 9340 9350
KVLARTKGSM LVSWTPPLDN GGSPITGYWL EKREEGSPYW SRVSRAPITK
            9360 9370 9380 9390 9400
VGLKGVEFNV PRLLEGVKYQ FRAMAINAAG IGPPSEPSDP EVAGDPIFPP
            9410 9420 9430 9440 9450
GPPSCPEVKD KTKSSISLGW KPPAKDGGSP IKGYIVEMQE EGTTDWKRVN
            9460 9470 9480 9490 9500
EPDKLITTCE CVVPNLKELR KYRFRVKAVN EAGESEPSDT TGEIPATDIQ
            9510 9520 9530 9540 9550
EEPEVFIDIG AQDCLVCKAG SQIRIPAVIK GRPTPKSSWE FDGKAKKAMK
            9560 9570 9580 9590 9600
DGVHDIPEDA QLETAENSSV IIIPECKRSH TGKYSITAKN KAGQKTANCR
            9610 9620 9630 9640 9650
VKVMDVPGPP KDLKVSDITR GSCRLSWKMP DDDGGDRIKG YVIEKRTIDG
            9660 9670 9680 9690 9700
KAWTKVNPDC GSTTFVVPDL LSEQQYFFRV RAENRFGIGP PVETIQRTTA
            9710 9720 9730 9740 9750
RDPIYPPDPP IKLKIGLITK NTVHLSWKPP KNDGGSPVTH YIVECLAWDP
            9760 9770 9780 9790 9800
TGTKKEAWRQ CNKRDVEELQ FTVEDLVEGG EYEFRVKAVN AAGVSKPSAT
            9810 9820 9830 9840 9850
VGPVTVKDQT CPPSIDLKEF MEVEEGTNVN IVAKIKGVPF PTLTWFKAPP
            9860 9870 9880 9890 9900
KKPDNKEPVL YDTHVNKLVV DDTCTLVIPQ SRRSDTGLYT ITAVNNLGTA
            9910 9920 9930 9940 9950
SKEMRLNVLG RPGPPVGPIK FESVSADQMT LSWFPPKDDG GSKITNYVIE
            9960 9970 9980 9990 10000
KREANRKTWV HVSSEPKECT YTIPKLLEGH EYVFRIMAQN KYGIGEPLDS
          10010 10020 10030 10040 10050
EPETARNLFS VPGAPDKPTV SSVTRNSMTV NWEEPEYDGG SPVTGYWLEM
          10060 10070 10080 10090 10100
KDTTSKRWKR VNRDPIKAMT LGVSYKVTGL IEGSDYQFRV YAINAAGVGP
          10110 10120 10130 10140 10150
ASLPSDPATA RDPIAPPGPP FPKVTDWTKS SADLEWSPPL KDGGSKVTGY
          10160 10170 10180 10190 10200
IVEYKEEGKE EWEKGKDKEV RGTKLVVTGL KEGAFYKFRV RAVNIAGIGE
          10210 10220 10230 10240 10250
PGEVTDVIEM KDRLVSPDLQ LDASVRDRIV VHAGGVIRII AYVSGKPPPT
          10260 10270 10280 10290 10300
VTWNMNERTL PQEATIETTA ISSSMVIKNC QRSHQGVYSL LAKNEAGERK
          10310 10320 10330 10340 10350
KTIIVDVLDV PGPVGTPFLA HNLTNESCKL TWFSPEDDGG SPITNYVIEK
          10360 10370 10380 10390 10400
RESDRRAWTP VTYTVTRQNA TVQGLIQGKA YFFRIAAENS IGMGPFVETS
          10410 10420 10430 10440 10450
EALVIREPIT VPERPEDLEV KEVTKNTVTL TWNPPKYDGG SEIINYVLES
          10460 10470 10480 10490 10500
RLIGTEKFHK VTNDNLLSRK YTVKGLKEGD TYEYRVSAVN IVGQGKPSFC
          10510 10520 10530 10540 10550
TKPITCKDEL APPTLHLDER DKLTIRVGEA FALTGRYSGK PKPKVSWFKD
          10560 10570 10580 10590 10600
EADVLEDDRT HIKTTPATLA LEKIKAKRSD SGKYCVVVEN STGSRKGFCQ
          10610 10620 10630 10640 10650
VNVVDRPGPP VGPVSFDEVT KDYMVISWKP PLDDGGSKIT NYIIEKKEVG
          10660 10670 10680 10690 10700
KDVWMPVTSA SAKTTCKVSK LLEGKDYIFR IHAENLYGIS DPLVSDSMKA
          10710 10720 10730 10740 10750
KDRFRVPDAP DQPIVTEVTK DSALVTWNKP HDGGKPITNY ILEKRETMSK
          10760 10770 10780 10790 10800
RWARVTKDPI HPYTKFRVPD LLEGCQYEFR VSAENEIGIG DPSPPSKPVF
          10810 10820 10830 10840 10850
AKDPIAKPSP PVNPEAIDTT CNSVDLTWQP PRHDGGSKIL GYIVEYQKVG
          10860 10870 10880 10890 10900
DEEWRRANHT PESCPETKYK VTGLRDGQTY KFRVLAVNAA GESDPAHVPE
          10910 10920 10930 10940 10950
PVLVKDRLEP PELILDANMA REQHIKVGDT LRLSAIIKGV PFPKVTWKKE
          10960 10970 10980 10990 11000
DRDAPTKARI DVTPVGSKLE IRNAAHEDGG IYSLTVENPA GSKTVSVKVL
          11010 11020 11030 11040 11050
VLDKPGPPRD LEVSEIRKDS CYLTWKEPLD DGGSVITNYV VERRDVASAQ
          11060 11070 11080 11090 11100
WSPLSATSKK KSHFAKHLNE GNQYLFRVAA ENQYGRGPFV ETPKPIKALD
          11110 11120 11130 11140 11150
PLHPPGPPKD LHHVDVDKTE VSLVWNKPDR DGGSPITGYL VEYQEEGTQD
          11160 11170 11180 11190 11200
WIKFKTVTNL ECVVTGLQQG KTYRFRVKAE NIVGLGLPDT TIPIECQEKL
          11210 11220 11230 11240 11250
VPPSVELDVK LIEGLVVKAG TTVRFPAIIR GVPVPTAKWT TDGSEIKTDE
          11260 11270 11280 11290 11300
HYTVETDNES SVLTIKNCLR RDTGEYQITV SNAAGSKTVA VHLTVLDVPG
          11310 11320 11330 11340 11350
PPTGPINILD VTPEHMTISW QPPKDDGGSP VINYIVEKQD TRKDTWGVVS
          11360 11370 11380 11390 11400
SGSSKTKLKI PHLQKGCEYV FRVRAENKIG VGPPLDSTPT VAKHKFSPPS
          11410 11420 11430 11440 11450
PPGKPVVTDI TENAATVSWT LPKSDGGSPI TGYYMERREV TGKWVRVNKT
          11460 11470 11480 11490 11500
PIADLKFRVT GLYEGNTYEF RVFAENLAGL SKPSPSSDPI KACRPIKPPG
          11510 11520 11530 11540 11550
PPINPKLKDK SRETADLVWT KPLSDGGSPI LGYVVECQKP GTAQWNRINK
          11560 11570 11580 11590 11600
DELIRQCAFR VPGLIEGNEY RFRIKAANIV GEGEPRELAE SVIAKDILHP
          11610 11620 11630 11640 11650
PEVELDVTCR DVITVRVGQT IRILARVKGR PEPDITWTKE GKVLVREKRV
          11660 11670 11680 11690 11700
DLIQDLPRVE LQIKEAVRAD HGKYIISAKN SSGHAQGSAI VNVLDRPGPC
          11710 11720 11730 11740 11750
QNLKVTNVTK ENCTISWENP LDNGGSEITN FIVEYRKPNQ KGWSIVASDV
          11760 11770 11780 11790 11800
TKRLIKANLL ANNEYYFRVC AENKVGVGPT IETKTPILAI NPIDRPGEPE
          11810 11820 11830 11840 11850
NLHIADKGKT FVYLKWRRPD YDGGSPNLSY HVERRLKGSD DWERVHKGSI
          11860 11870 11880 11890 11900
KETHYMVDRC VENQIYEFRV QTKNEGGESD WVKTEEVVVK EDLQKPVLDL
          11910 11920 11930 11940 11950
KLSGVLTVKA GDTIRLEAGV RGKPFPEVAW TKDKDATDLT RSPRVKIDTR
          11960 11970 11980 11990 12000
ADSSKFSLTK AKRSDGGKYV VTATNTAGSF VAYATVNVLD KPGPVRNLKI
          12010 12020 12030 12040 12050
VDVSSDRCTV CWDPPEDDGG CEIQNYILEK CETKRMVWST YSATVLTPGT
          12060 12070 12080 12090 12100
TVTRLIEGNE YIFRVRAENK IGTGPPTESK PVIAKTKYDK PGRPDPPEVT
          12110 12120 12130 12140 12150
KVSKEEMTVV WNPPEYDGGK SITGYFLEKK EKHSTRWVPV NKSAIPERRM
          12160 12170 12180 12190 12200
KVQNLLPDHE YQFRVKAENE IGIGEPSLPS RPVVAKDPIE PPGPPTNFRV
          12210 12220 12230 12240 12250
VDTTKHSITL GWGKPVYDGG APIIGYVVEM RPKIADASPD EGWKRCNAAA
          12260 12270 12280 12290 12300
QLVRKEFTVT SLDENQEYEF RVCAQNQVGI GRPAELKEAI KPKEILEPPE
          12310 12320 12330 12340 12350
IDLDASMRKL VIVRAGCPIR LFAIVRGRPA PKVTWRKVGI DNVVRKGQVD
          12360 12370 12380 12390 12400
LVDTMAFLVI PNSTRDDSGK YSLTLVNPAG EKAVFVNVRV LDTPGPVSDL
          12410 12420 12430 12440 12450
KVSDVTKTSC HVSWAPPEND GGSQVTHYIV EKREADRKTW STVTPEVKKT
          12460 12470 12480 12490 12500
SFHVTNLVPG NEYYFRVTAV NEYGPGVPTD VPKPVLASDP LSEPDPPRKL
          12510 12520 12530 12540 12550
EVTEMTKNSA TLAWLPPLRD GGAKIDGYIT SYREEEQPAD RWTEYSVVKD
          12560 12570 12580 12590 12600
LSLVVTGLKE GKKYKFRVAA RNAVGVSLPR EAEGVYEAKE QLLPPKILMP
          12610 12620 12630 12640 12650
EQITIKAGKK LRIEAHVYGK PHPTCKWKKG EDEVVTSSHL AVHKADSSSI
          12660 12670 12680 12690 12700
LIIKDVTRKD SGYYSLTAEN SSGTDTQKIK VVVMDAPGPP QPPFDISDID
          12710 12720 12730 12740 12750
ADACSLSWHI PLEDGGSNIT NYIVEKCDVS RGDWVTALAS VTKTSCRVGK
          12760 12770 12780 12790 12800
LIPGQEYIFR VRAENREGIS EPLTSPKMVA QFPFGVPSEP KNARVTKVNK
          12810 12820 12830 12840 12850
DCIFVAWDRP DSDGGSPIIG YLIERKERNS LLWVKANDTL VRSTEYPCAG
          12860 12870 12880 12890 12900
LVEGLEYSFR IYALNKAGSS PPSKPTEYVT ARMPVDPPGK PEVIDVTKST
          12910 12920 12930 12940 12950
VSLIWARPKH DGGSKIIGYF VEACKLPGDK WVRCNTAPHQ IPQEEYTATG
          12960 12970 12980 12990 13000
LEEKAQYQFR AIARTAVNIS PPSEPSDPVT ILAENVPPRI DLSVAMKSLL
          13010 13020 13030 13040 13050
TVKAGTNVCL DATVFGKPMP TVSWKKDGTL LKPAEGIKMA MQRNLCTLEL
          13060 13070 13080 13090 13100
FSVNRKDSGD YTITAENSSG SKSATIKLKV LDKPGPPASV KINKMYSDRA
          13110 13120 13130 13140 13150
MLSWEPPLED GGSEITNYIV DKRETSRPNW AQVSATVPIT SCSVEKLIEG
          13160 13170 13180 13190 13200
HEYQFRICAE NKYGVGDPVF TEPAIAKNPY DPPGRCDPPV ISNITKDHMT
          13210 13220 13230 13240 13250
VSWKPPADDG GSPITGYLLE KRETQAVNWT KVNRKPIIER TLKATGLQEG
          13260 13270 13280 13290 13300
TEYEFRVTAI NKAGPGKPSD ASKAAYARDP QYPPGPPAFP KVYDTTRSSV
          13310 13320 13330 13340 13350
SLSWGKPAYD GGSPIIGYLV EVKRADSDNW VRCNLPQNLQ KTRFEVTGLM
          13360 13370 13380 13390 13400
EDTQYQFRVY AVNKIGYSDP SDVPDKHYPK DILIPPEGEL DADLRKTLIL
          13410 13420 13430 13440 13450
RAGVTMRLYV PVKGRPPPKI TWSKPNVNLR DRIGLDIKST DFDTFLRCEN
          13460 13470 13480 13490 13500
VNKYDAGKYI LTLENSCGKK EYTIVVKVLD TPGPPVNVTV KEISKDSAYV
          13510 13520 13530 13540 13550
TWEPPIIDGG SPIINYVVQK RDAERKSWST VTTECSKTSF RVANLEEGKS
          13560 13570 13580 13590 13600
YFFRVFAENE YGIGDPGETR DAVKASQTPG PVVDLKVRSV SKSSCSIGWK
          13610 13620 13630 13640 13650
KPHSDGGSRI IGYVVDFLTE ENKWQRVMKS LSLQYSAKDL TEGKEYTFRV
          13660 13670 13680 13690 13700
SAENENGEGT PSEITVVARD DVVAPDLDLK GLPDLCYLAK ENSNFRLKIP
          13710 13720 13730 13740 13750
IKGKPAPSVS WKKGEDPLAT DTRVSVESSA VNTTLIVYDC QKSDAGKYTI
          13760 13770 13780 13790 13800
TLKNVAGTKE GTISIKVVGK PGIPTGPIKF DEVTAEAMTL KWAPPKDDGG
          13810 13820 13830 13840 13850
SEITNYILEK RDSVNNKWVT CASAVQKTTF RVTRLHEGME YTFRVSAENK
          13860 13870 13880 13890 13900
YGVGEGLKSE PIVARHPFDV PDAPPPPNIV DVRHDSVSLT WTDPKKTGGS
          13910 13920 13930 13940 13950
PITGYHLEFK ERNSLLWKRA NKTPIRMRDF KVTGLTEGLE YEFRVMAINL
          13960 13970 13980 13990 14000
AGVGKPSLPS EPVVALDPID PPGKPEVINI TRNSVTLIWT EPKYDGGHKL
          14010 14020 14030 14040 14050
TGYIVEKRDL PSKSWMKANH VNVPECAFTV TDLVEGGKYE FRIRAKNTAG
          14060 14070 14080 14090 14100
AISAPSESTE TIICKDEYEA PTIVLDPTIK DGLTIKAGDT IVLNAISILG
          14110 14120 14130 14140 14150
KPLPKSSWSK AGKDIRPSDI TQITSTPTSS MLTIKYATRK DAGEYTITAT
          14160 14170 14180 14190 14200
NPFGTKVEHV KVTVLDVPGP PGPVEISNVS AEKATLTWTP PLEDGGSPIK
          14210 14220 14230 14240 14250
SYILEKRETS RLLWTVVSED IQSCRHVATK LIQGNEYIFR VSAVNHYGKG
          14260 14270 14280 14290 14300
EPVQSEPVKM VDRFGPPGPP EKPEVSNVTK NTATVSWKRP VDDGGSEITG
          14310 14320 14330 14340 14350
YHVERREKKS LRWVRAIKTP VSDLRCKVTG LQEGSTYEFR VSAENRAGIG
          14360 14370 14380 14390 14400
PPSEASDSVL MKDAAYPPGP PSNPHVTDTT KKSASLAWGK PHYDGGLEIT
          14410 14420 14430 14440 14450
GYVVEHQKVG DEAWIKDTTG TALRITQFVV PDLQTKEKYN FRISAINDAG
          14460 14470 14480 14490 14500
VGEPAVIPDV EIVEREMAPD FELDAELRRT LVVRAGLSIR IFVPIKGRPA
          14510 14520 14530 14540 14550
PEVTWTKDNI NLKNRANIEN TESFTLLIIP ECNRYDTGKF VMTIENPAGK
          14560 14570 14580 14590 14600
KSGFVNVRVL DTPGPVLNLR PTDITKDSVT LHWDLPLIDG GSRITNYIVE
          14610 14620 14630 14640 14650
KREATRKSYS TATTKCHKCT YKVTGLSEGC EYFFRVMAEN EYGIGEPTET
          14660 14670 14680 14690 14700
TEPVKASEAP SPPDSLNIMD ITKSTVSLAW PKPKHDGGSK ITGYVIEAQR
          14710 14720 14730 14740 14750
KGSDQWTHIT TVKGLECVVR NLTEGEEYTF QVMAVNSAGR SAPRESRPVI
          14760 14770 14780 14790 14800
VKEQTMLPEL DLRGIYQKLV IAKAGDNIKV EIPVLGRPKP TVTWKKGDQI
          14810 14820 14830 14840 14850
LKQTQRVNFE TTATSTILNI NECVRSDSGP YPLTARNIVG EVGDVITIQV
          14860 14870 14880 14890 14900
HDIPGPPTGP IKFDEVSSDF VTFSWDPPEN DGGVPISNYV VEMRQTDSTT
          14910 14920 14930 14940 14950
WVELATTVIR TTYKATRLTT GLEYQFRVKA QNRYGVGPGI TSACIVANYP
          14960 14970 14980 14990 15000
FKVPGPPGTP QVTAVTKDSM TISWHEPLSD GGSPILGYHV ERKERNGILW
          15010 15020 15030 15040 15050
QTVSKALVPG NIFKSSGLTD GIAYEFRVIA ENMAGKSKPS KPSEPMLALD
          15060 15070 15080 15090 15100
PIDPPGKPVP LNITRHTVTL KWAKPEYTGG FKITSYIVEK RDLPNGRWLK
          15110 15120 15130 15140 15150
ANFSNILENE FTVSGLTEDA AYEFRVIAKN AAGAISPPSE PSDAITCRDD
          15160 15170 15180 15190 15200
VEAPKIKVDV KFKDTVILKA GEAFRLEADV SGRPPPTMEW SKDGKELEGT
          15210 15220 15230 15240 15250
AKLEIKIADF STNLVNKDST RRDSGAYTLT ATNPGGFAKH IFNVKVLDRP
          15260 15270 15280 15290 15300
GPPEGPLAVT EVTSEKCVLS WFPPLDDGGA KIDHYIVQKR ETSRLAWTNV
          15310 15320 15330 15340 15350
ASEVQVTKLK VTKLLKGNEY IFRVMAVNKY GVGEPLESEP VLAVNPYGPP
          15360 15370 15380 15390 15400
DPPKNPEVTT ITKDSMVVCW GHPDSDGGSE IINYIVERRD KAGQRWIKCN
          15410 15420 15430 15440 15450
KKTLTDLRYK VSGLTEGHEY EFRIMAENAA GISAPSPTSP FYKACDTVFK
          15460 15470 15480 15490 15500
PGPPGNPRVL DTSRSSISIA WNKPIYDGGS EITGYMVEIA LPEEDEWQIV
          15510 15520 15530 15540 15550
TPPAGLKATS YTITGLTENQ EYKIRIYAMN SEGLGEPALV PGTPKAEDRM
          15560 15570 15580 15590 15600
LPPEIELDAD LRKVVTIRAC CTLRLFVPIK GRPAPEVKWA RDHGESLDKA
          15610 15620 15630 15640 15650
SIESTSSYTL LIVGNVNRFD SGKYILTVEN SSGSKSAFVN VRVLDTPGPP
          15660 15670 15680 15690 15700
QDLKVKEVTK TSVTLTWDPP LLDGGSKIKN YIVEKRESTR KAYSTVATNC
          15710 15720 15730 15740 15750
HKTSWKVDQL QEGCSYYFRV LAENEYGIGL PAETAESVKA SERPLPPGKI
          15760 15770 15780 15790 15800
TLMDVTRNSV SLSWEKPEHD GGSRILGYIV EMQTKGSDKW ATCATVKVTE
          15810 15820 15830 15840 15850
ATITGLIQGE EYSFRVSAQN EKGISDPRQL SVPVIAKDLV IPPAFKLLEN
          15860 15870 15880 15890 15900
TFTVLAGEDL KVDVPFIGRP TPAVTWHKDN VPLKQTTRVN AESTENNSLL
          15910 15920 15930 15940 15950
TIKDACREDV GHYVVKLINS AGEAIETLNV IVLDKPGPPT GPVKMDEVTA
          15960 15970 15980 15990 16000
DSITLSWGPP KYDGGSSINN YIVEKRDTST TTWQIVSATV ARTTIKACRL
          16010 16020 16030 16040 16050
KTGCEYQFRI AAENRYGKST YLNSEPTVAQ YPFKVPGPPG TPVVTLSSRD
          16060 16070 16080 16090 16100
SMEVQWNEPI SDGGSRVIGY HLERKERNSI LWVKLNKTPI PQTKFKTTGL
          16110 16120 16130 16140 16150
EEGVEYEFRV SAENIVGIGK PSKVSECYVA RDPCDPPGRP EAIIVTRNSV
          16160 16170 16180 16190 16200
TLQWKKPTYD GGSKITGYIV EKKELPEGRW MKASFTNIID THFEVTGLVE
          16210 16220 16230 16240 16250
DHRYEFRVIA RNAAGVFSEP SESTGAITAR DEVDPPRISM DPKYKDTIVV
          16260 16270 16280 16290 16300
HAGESFKVDA DIYGKPIPTI QWIKGDQELS NTARLEIKST DFATSLSVKD
          16310 16320 16330 16340 16350
AVRVDSGNYI LKAKNVAGER SVTVNVKVLD RPGPPEGPVV ISGVTAEKCT
          16360 16370 16380 16390 16400
LAWKPPLQDG GSDIINYIVE RRETSRLVWT VVDANVQTLS CKVTKLLEGN
          16410 16420 16430 16440 16450
EYTFRIMAVN KYGVGEPLES EPVVAKNPFV VPDAPKAPEV TTVTKDSMIV
          16460 16470 16480 16490 16500
VWERPASDGG SEILGYVLEK RDKEGIRWTR CHKRLIGELR LRVTGLIENH
          16510 16520 16530 16540 16550
DYEFRVSAEN AAGLSEPSPP SAYQKACDPI YKPGPPNNPK VIDITRSSVF
          16560 16570 16580 16590 16600
LSWSKPIYDG GCEIQGYIVE KCDVSVGEWT MCTPPTGINK TNIEVEKLLE
          16610 16620 16630 16640 16650
KHEYNFRICA INKAGVGEHA DVPGPIIVEE KLEAPDIDLD LELRKIINIR
          16660 16670 16680 16690 16700
AGGSLRLFVP IKGRPTPEVK WGKVDGEIRD AAIIDVTSSF TSLVLDNVNR
          16710 16720 16730 16740 16750
YDSGKYTLTL ENSSGTKSAF VTVRVLDTPS PPVNLKVTEI TKDSVSITWE
          16760 16770 16780 16790 16800
PPLLDGGSKI KNYIVEKREA TRKSYAAVVT NCHKNSWKID QLQEGCSYYF
          16810 16820 16830 16840 16850
RVTAENEYGI GLPAQTADPI KVAEVPQPPG KITVDDVTRN SVSLSWTKPE
          16860 16870 16880 16890 16900
HDGGSKIIQY IVEMQAKHSE KWSECARVKS LQAVITNLTQ GEEYLFRVVA
          16910 16920 16930 16940 16950
VNEKGRSDPR SLAVPIVAKD LVIEPDVKPA FSSYSVQVGQ DLKIEVPISG
          16960 16970 16980 16990 17000
RPKPTITWTK DGLPLKQTTR INVTDSLDLT TLSIKETHKD DGGQYGITVA
          17010 17020 17030 17040 17050
NVVGQKTASI EIVTLDKPDP PKGPVKFDDV SAESITLSWN PPLYTGGCQI
          17060 17070 17080 17090 17100
TNYIVQKRDT TTTVWDVVSA TVARTTLKVT KLKTGTEYQF RIFAENRYGQ
          17110 17120 17130 17140 17150
SFALESDPIV AQYPYKEPGP PGTPFATAIS KDSMVIQWHE PVNNGGSPVI
          17160 17170 17180 17190 17200
GYHLERKERN SILWTKVNKT IIHDTQFKAQ NLEEGIEYEF RVYAENIVGV
          17210 17220 17230 17240 17250
GKASKNSECY VARDPCDPPG TPEPIMVKRN EITLQWTKPV YDGGSMITGY
          17260 17270 17280 17290 17300
IVEKRDLPDG RWMKASFTNV IETQFTVSGL TEDQRYEFRV IAKNAAGAIS
          17310 17320 17330 17340 17350
KPSDSTGPIT AKDEVELPRI SMDPKFRDTI VVNAGETFRL EADVHGKPLP
          17360 17370 17380 17390 17400
TIEWLRGDKE IEESARCEIK NTDFKALLIV KDAIRIDGGQ YILRASNVAG
          17410 17420 17430 17440 17450
SKSFPVNVKV LDRPGPPEGP VQVTGVTSEK CSLTWSPPLQ DGGSDISHYV
          17460 17470 17480 17490 17500
VEKRETSRLA WTVVASEVVT NSLKVTKLLE GNEYVFRIMA VNKYGVGEPL
          17510 17520 17530 17540 17550
ESAPVLMKNP FVLPGPPKSL EVTNIAKDSM TVCWNRPDSD GGSEIIGYIV
          17560 17570 17580 17590 17600
EKRDRSGIRW IKCNKRRITD LRLRVTGLTE DHEYEFRVSA ENAAGVGEPS
          17610 17620 17630 17640 17650
PATVYYKACD PVFKPGPPTN AHIVDTTKNS ITLAWGKPIY DGGSEILGYV
          17660 17670 17680 17690 17700
VEICKADEEE WQIVTPQTGL RVTRFEISKL TEHQEYKIRV CALNKVGLGE
          17710 17720 17730 17740 17750
ATSVPGTVKP EDKLEAPELD LDSELRKGIV VRAGGSARIH IPFKGRPTPE
          17760 17770 17780 17790 17800
ITWSREEGEF TDKVQIEKGV NYTQLSIDNC DRNDAGKYIL KLENSSGSKS
          17810 17820 17830 17840 17850
AFVTVKVLDT PGPPQNLAVK EVRKDSAFLV WEPPIIDGGA KVKNYVIDKR
          17860 17870 17880 17890 17900
ESTRKAYANV SSKCSKTSFK VENLTEGAIY YFRVMAENEF GVGVPVETVD
          17910 17920 17930 17940 17950
AVKAAEPPSP PGKVTLTDVS QTSASLMWEK PEHDGGSRVL GYVVEMQPKG
          17960 17970 17980 17990 18000
TEKWSIVAES KVCNAVVTGL SSGQEYQFRV KAYNEKGKSD PRVLGVPVIA
          18010 18020 18030 18040 18050
KDLTIQPSLK LPFNTYSIQA GEDLKIEIPV IGRPRPNISW VKDGEPLKQT
          18060 18070 18080 18090 18100
TRVNVEETAT STVLHIKEGN KDDFGKYTVT ATNSAGTATE NLSVIVLEKP
          18110 18120 18130 18140 18150
GPPVGPVRED EVSADFVVIS WEPPAYTGGC QISNYIVEKR DTTTTTWHMV
          18160 18170 18180 18190 18200
SATVARTTIK ITKLKTGTEY QFRIFAENRY GKSAPLDSKA VIVQYPFKEP
          18210 18220 18230 18240 18250
GPPGTPFVTS ISKDQMLVQW HEPVNDGGTK IIGYHLEQKE KNSILWVKLN
          18260 18270 18280 18290 18300
KTPIQDTKFK TTGLDEGLEY EFKVSAENIV GIGKPSKVSE CFVARDPCDP
          18310 18320 18330 18340 18350
PGRPEAIVIT RNNVTLKWKK PAYDGGSKIT GYIVEKKDLP DGRWMKASFT
          18360 18370 18380 18390 18400
NVLETEFTVS GLVEDQRYEF RVIARNAAGN FSEPSDSSGA ITARDEIDAP
          18410 18420 18430 18440 18450
NASLDPKYKD VIVVHAGETF VLEADIRGKP IPDVVWSKDG KELEETAARM
          18460 18470 18480 18490 18500
EIKSTIQKTT LVVKDCIRTD GGQYILKLSN VGGTKSIPIT VKVLDRPGPP
          18510 18520 18530 18540 18550
EGPLKVTGVT AEKCYLAWNP PLQDGGANIS HYIIEKRETS RLSWTQVSTE
          18560 18570 18580 18590 18600
VQALNYKVTK LLPGNEYIFR VMAVNKYGIG EPLESGPVTA CNPYKPPGPP
          18610 18620 18630 18640 18650
STPEVSAITK DSMVVTWARP VDDGGTEIEG YILEKRDKEG VRWTKCNKKT
          18660 18670 18680 18690 18700
LTDLRLRVTG LTEGHSYEFR VAAENAAGVG EPSEPSVFYR ACDALYPPGP
          18710 18720 18730 18740 18750
PSNPKVTDTS RSSVSLAWSK PIYDGGAPVK GYVVEVKEAA ADEWTTCTPP
          18760 18770 18780 18790 18800
TGLQGKQFTV TKLKENTEYN FRICAINSEG VGEPATLPGS VVAQERIEPP
          18810 18820 18830 18840 18850
EIELDADLRK VVVLRASATL RLFVTIKGRP EPEVKWEKAE GILTDRAQIE
          18860 18870 18880 18890 18900
VTSSFTMLVI DNVTRFDSGR YNLTLENNSG SKTAFVNVRV LDSPSAPVNL
          18910 18920 18930 18940 18950
TIREVKKDSV TLSWEPPLID GGAKITNYIV EKRETTRKAY ATITNNCTKT
          18960 18970 18980 18990 19000
TFRIENLQEG CSYYFRVLAS NEYGIGLPAE TTEPVKVSEP PLPPGRVTLV
          19010 19020 19030 19040 19050
DVTRNTATIK WEKPESDGGS KITGYVVEMQ TKGSEKWSTC TQVKTLEATI
          19060 19070 19080 19090 19100
SGLTAGEEYV FRVAAVNEKG RSDPRQLGVP VIARDIEIKP SVELPFHTEN
          19110 19120 19130 19140 19150
VKAREQLKID VPFKGRPQAT VNWRKDGQTL KETTRVNVSS SKTVTSLSIK
          19160 19170 19180 19190 19200
EASKEDVGTY ELCVSNSAGS ITVPITIIVL DRPGPPGPIR IDEVSCDSIT
          19210 19220 19230 19240 19250
ISWNPPEYDG GCQISNYIVE KKETTSTTWH IVSQAVARTS IKIVRLTTGS
          19260 19270 19280 19290 19300
EYQFRVCAEN RYGKSSYSES SAVVAEYPFS PPGPPGTPKV VHATKSTMLV
          19310 19320 19330 19340 19350
TWQVPVNDGG SRVIGYHLEY KERSSILWSK ANKILIADTQ MKVSGLDEGL
          19360 19370 19380 19390 19400
MYEYRVYAEN IAGIGKCSKS CEPVPARDPC DPPGQPEVTN ITRKSVSLKW
          19410 19420 19430 19440 19450
SKPHYDGGAK ITGYIVERRE LPDGRWLKCN YTNIQETYFE VTELTEDQRY
          19460 19470 19480 19490 19500
EFRVFARNAA DSVSEPSEST GPIIVKDDVE PPRVMMDVKF RDVIVVKAGE
          19510 19520 19530 19540 19550
VLKINADIAG RPLPVISWAK DGIEIEERAR TEIISTDNHT LLTVKDCIRR
          19560 19570 19580 19590 19600
DTGQYVLTLK NVAGTRSVAV NCKVLDKPGP PAGPLEINGL TAEKCSLSWG
          19610 19620 19630 19640 19650
RPQEDGGADI DYYIVEKRET SHLAWTICEG ELQMTSCKVT KLLKGNEYIF
          19660 19670 19680 19690 19700
RVTGVNKYGV GEPLESVAIK ALDPFTVPSP PTSLEITSVT KESMTLCWSR
          19710 19720 19730 19740 19750
PESDGGSEIS GYIIERREKN SLRWVRVNKK PVYDLRVKST GLREGCEYEY
          19760 19770 19780 19790 19800
RVYAENAAGL SLPSETSPLI RAEDPVFLPS PPSKPKIVDS GKTTITIAWV
          19810 19820 19830 19840 19850
KPLFDGGAPI TGYTVEYKKS DDTDWKTSIQ SLRGTEYTIS GLTTGAEYVF
          19860 19870 19880 19890 19900
RVKSVNKVGA SDPSDSSDPQ IAKEREEEPL FDIDSEMRKT LIVKAGASFT
          19910 19920 19930 19940 19950
MTVPFRGRPV PNVLWSKPDT DLRTRAYVDT TDSRTSLTIE NANRNDSGKY
          19960 19970 19980 19990 20000
TLTIQNVLSA ASLTLVVKVL DTPGPPTNIT VQDVTKESAV LSWDVPENDG
          20010 20020 20030 20040 20050
GAPVKNYHIE KREASKKAWV SVINNCNRLS YKVTNLQEGA IYYFRVSGEN
          20060 20070 20080 20090 20100
EFGVGIPAET KEGVKITEKP SPPEKLGVTS ISKDSVSLTW LKPEHDGGSR
          20110 20120 20130 20140 20150
IVHYVVEALE KGQKNWVKCA VAKSTHHVVS GLRENSEYFF RVFAENQAGL
          20160 20170 20180 20190 20200
SDPRELLLPV LIKEQLEPPE IDMKNFPSHT VYVRAGSNLK VDIPISGKPL
          20210 20220 20230 20240 20250
PKVTLSRDGV PLKATMRENT EITAENLTIN LKESVTADAG RYEITAANSS
          20260 20270 20280 20290 20300
GTTKAFINIV VLDRPGPPTG PVVISDITEE SVTLKWEPPK YDGGSQVTNY
          20310 20320 20330 20340 20350
ILLKRETSTA VWTEVSATVA RTMMKVMKLT TGEEYQFRIK AENREGISDH
          20360 20370 20380 20390 20400
IDSACVTVKL PYTTPGPPST PWVTNVTRES ITVGWHEPVS NGGSAVVGYH
          20410 20420 20430 20440 20450
LEMKDRNSIL WQKANKLVIR TTHFKVTTIS AGLIYEFRVY AENAAGVGKP
          20460 20470 20480 20490 20500
SHPSEPVLAI DACEPPRNVR ITDISKNSVS LSWQQPAFDG GSKITGYIVE
          20510 20520 20530 20540 20550
RRDLPDGRWT KASFTNVTET QFIISGLTQN SQYEFRVFAR NAVGSISNPS
          20560 20570 20580 20590 20600
EVVGPITCID SYGGPVIDLP LEYTEVVKYR AGTSVKLRAG ISGKPAPTIE
          20610 20620 20630 20640 20650
WYKDDKELQT NALVCVENTT DLASILIKDA DRINSGCYEL KLRNAMGSAS
          20660 20670 20680 20690 20700
ATIRVQILDK PGPPGGPIEF KTVTAEKITL LWRPPADDGG AKITHYIVEK
          20710 20720 20730 20740 20750
RETSRVVWSM VSEHLEECII TTTKIIKGNE YIFRVRAVNK YGIGEPLESD
          20760 20770 20780 20790 20800
SVVAKNAFVT PGPPGIPEVT KITKNSMTVV WSRPIADGGS DISGYFLEKR
          20810 20820 20830 20840 20850
DKKSLGWFKV LKETIRDTRQ KVTGLTENSD YQYRVCAVNA AGQGPFSEPS
          20860 20870 20880 20890 20900
EFYKAADPID PPGPPAKIRI ADSTKSSITL GWSKPVYDGG SAVTGYVVEI
          20910 20920 20930 20940 20950
RQGEEEEWTT VSTKGEVRTT EYVVSNLKPG VNYYFRVSAV NCAGQGEPIE
          20960 20970 20980 20990 21000
MNEPVQAKDI LEAPEIDLDV ALRTSVIAKA GEDVQVLIPF KGRPPPTVTW
          21010 21020 21030 21040 21050
RKDEKNLGSD ARYSIENTDS SSLLTIPQVT RNDTGKYILT IENGVGEPKS
          21060 21070 21080 21090 21100
STVSVKVLDT PAACQKLQVK HVSRGTVTLL WDPPLIDGGS PIINYVIEKR
          21110 21120 21130 21140 21150
DATKRTWSVV SHKCSSTSFK LIDLSEKTPF FFRVLAENEI GIGEPCETTE
          21160 21170 21180 21190 21200
PVKAAEVPAP IRDLSMKDST KTSVILSWTK PDFDGGSVIT EYVVERKGKG
          21210 21220 21230 21240 21250
EQTWSHAGIS KTCEIEVSQL KEQSVLEFRV FAKNEKGLSD PVTIGPITVK
          21260 21270 21280 21290 21300
ELIITPEVDL SDIPGAQVTV RIGHNVHLEL PYKGKPKPSI SWLKDGLPLK
          21310 21320 21330 21340 21350
ESEFVRFSKT ENKITLSIKN AKKEHGGKYT VILDNAVCRI AVPITVITLG
          21360 21370 21380 21390 21400
PPSKPKGPIR FDEIKADSVI LSWDVPEDNG GGEITCYSIE KRETSQTNWK
          21410 21420 21430 21440 21450
MVCSSVARTT FKVPNLVKDA EYQFRVRAEN RYGVSQPLVS SIIVAKHQFR
          21460 21470 21480 21490 21500
IPGPPGKPVI YNVTSDGMSL TWDAPVYDGG SEVTGFHVEK KERNSILWQK
          21510 21520 21530 21540 21550
VNTSPISGRE YRATGLVEGL DYQFRVYAEN SAGLSSPSDP SKFTLAVSPV
          21560 21570 21580 21590 21600
DPPGTPDYID VTRETITLKW NPPLRDGGSK IVGYSIEKRQ GNERWVRCNF
          21610 21620 21630 21640 21650
TDVSECQYTV TGLSPGDRYE FRIIARNAVG TISPPSQSSG IIMTRDENVP
          21660 21670 21680 21690 21700
PIVEFGPEYF DGLIIKSGES LRIKALVQGR PVPRVTWFKD GVEIEKRMNM
          21710 21720 21730 21740 21750
EITDVLGSTS LFVRDATRDH RGVYTVEAKN ASGSAKAEIK VKVQDTPGKV
          21760 21770 21780 21790 21800
VGPIRFTNIT GEKMTLWWDA PLNDGCAPIT HYIIEKRETS RLAWALIEDK
          21810 21820 21830 21840 21850
CEAQSYTAIK LINGNEYQFR VSAVNKFGVG RPLDSDPVVA QIQYTVPDAP
          21860 21870 21880 21890 21900
GIPEPSNITG NSITLTWARP ESDGGSEIQQ YILERREKKS TRWVKVISKR
          21910 21920 21930 21940 21950
PISETRFKVT GLTEGNEYEF HVMAENAAGV GPASGISRLI KCREPVNPPG
          21960 21970 21980 21990 22000
PPTVVKVTDT SKTTVSLEWS KPVEDGGMEI IGYIIEMCKA DLGDWHKVNA
          22010 22020 22030 22040 22050
EACVKTRYTV TDLQAGEEYK FRVSAINGAG KGDSCEVTGT IKAVDRLTAP
          22060 22070 22080 22090 22100
ELDIDANFKQ THVVRAGASI RLFIAYQGRP TPTAVWSKPD SNLSLRADIH
          22110 22120 22130 22140 22150
TTDSFSTLTV ENCNRNDAGK YTLTVENNSG SKSITFTVKV LDTPGPPGPI
          22160 22170 22180 22190 22200
TFKDVTRGSA TLMWDAPLLD GGARIHHYVV EKREASRRSW QVISEKCTRQ
          22210 22220 22230 22240 22250
IFKVNDLAEG VPYYFRVSAV NEYGVGEPYE MPEPIVATEQ PAPPRRLDVV
          22260 22270 22280 22290 22300
DTSKSSAVLA WLKPDHDGGS RITGYLLEMR QKGSDFWVEA GHTKQLTFTV
          22310 22320 22330 22340 22350
ERLVEKTEYE FRVKAKNDAG YSEPREAFSS VIIKEPQIEP TADLTGITNQ
          22360 22370 22380 22390 22400
LITCKAGSPF TIDVPISGRP APKVTWKLEE MRLKETDRVS ITTTKDRTTL
          22410 22420 22430 22440 22450
TVKDSMRGDS GRYFLTLENT AGVKTFSVTV VVIGRPGPVT GPIEVSSVSA
          22460 22470 22480 22490 22500
ESCVLSWGEP KDGGGTEITN YIVEKRESGT TAWQLVNSSV KRTQIKVTHL
          22510 22520 22530 22540 22550
TKYMEYSFRV SSENRFGVSK PLESAPIIAE HPFVPPSAPT RPEVYHVSAN
          22560 22570 22580 22590 22600
AMSIRWEEPY HDGGSKIIGY WVEKKERNTI LWVKENKVPC LECNYKVTGL
          22610 22620 22630 22640 22650
VEGLEYQFRT YALNAAGVSK ASEASRPIMA QNPVDAPGRP EVTDVTRSTV
          22660 22670 22680 22690 22700
SLIWSAPAYD GGSKVVGYII ERKPVSEVGD GRWLKCNYTI VSDNFFTVTA
          22710 22720 22730 22740 22750
LSEGDTYEFR VLAKNAAGVI SKGSESTGPV TCRDEYAPPK AELDARLHGD
          22760 22770 22780 22790 22800
LVTIRAGSDL VLDAAVGGKP EPKIIWTKGD KELDLCEKVS LQYTGKRATA
          22810 22820 22830 22840 22850
VIKFCDRSDS GKYTLTVKNA SGTKAVSVMV KVLDSPGPCG KLTVSRVTQE
          22860 22870 22880 22890 22900
KCTLAWSLPQ EDGGAEITHY IVERRETSRL NWVIVEGECP TLSYVVTRLI
          22910 22920 22930 22940 22950
KNNEYIFRVR AVNKYGPGVP VESEPIVARN SFTIPSPPGI PEEVGTGKEH
          22960 22970 22980 22990 23000
IIIQWTKPES DGGNEISNYL VDKREKKSLR WTRVNKDYVV YDTRLKVTSL
          23010 23020 23030 23040 23050
MEGCDYQFRV TAVNAAGNSE PSEASNFISC REPSYTPGPP SAPRVVDTTK
          23060 23070 23080 23090 23100
HSISLAWTKP MYDGGTDIVG YVLEMQEKDT DQWYRVHTNA TIRNTEFTVP
          23110 23120 23130 23140 23150
DLKMGQKYSF RVAAVNVKGM SEYSESIAEI EPVERIEIPD LELADDLKKT
          23160 23170 23180 23190 23200
VTIRAGASLR LMVSVSGRPP PVITWSKQGI DLASRAIIDT TESYSLLIVD
          23210 23220 23230 23240 23250
KVNRYDAGKY TIEAENQSGK KSATVLVKVY DTPGPCPSVK VKEVSRDSVT
          23260 23270 23280 23290 23300
ITWEIPTIDG GAPVNNYIVE KREAAMRAFK TVTTKCSKTL YRISGLVEGT
          23310 23320 23330 23340 23350
MYYFRVLPEN IYGIGEPCET SDAVLVSEVP LVPAKLEVVD VTKSTVTLAW
          23360 23370 23380 23390 23400
EKPLYDGGSR LTGYVLEACK AGTERWMKVV TLKPTVLEHT VTSLNEGEQY
          23410 23420 23430 23440 23450
LFRIRAQNEK GVSEPRETVT AVTVQDLRVL PTIDLSTMPQ KTIHVPAGRP
          23460 23470 23480 23490 23500
VELVIPIAGR PPPAASWFFA GSKLRESERV TVETHTKVAK LTIRETTIRD
          23510 23520 23530 23540 23550
TGEYTLELKN VTGTTSETIK VIILDKPGPP TGPIKIDEID ATSITISWEP
          23560 23570 23580 23590 23600
PELDGGAPLS GYVVEQRDAH RPGWLPVSES VTRSTFKFTR LTEGNEYVER
          23610 23620 23630 23640 23650
VAATNRFGIG SYLQSEVIEC RSSIRIPGPP ETLQIFDVSR DGMTLTWYPP
          23660 23670 23680 23690 23700
EDDGGSQVTG YIVERKEVRA DRWVRVNKVP VTMTRYRSTG LTEGLEYEHR
          23710 23720 23730 23740 23750
VTAINARGSG KPSRPSKPIV AMDPIAPPGK PQNPRVTDTT RTSVSLAWSV
          23760 23770 23780 23790 23800
PEDEGGSKVT GYLIEMQKVD QHEWTKCNTT PTKIREYTLT HLPQGAEYRF
          23810 23820 23830 23840 23850
RVLACNAGGP GEPAEVPGTV KVTEMLEYPD YELDERYQEG IFVRQGGVIR
          23860 23870 23880 23890 23900
LTIPIKGKPF PICKWTKEGQ DISKRAMIAT SETHTELVIK EADRGDSGTY
          23910 23920 23930 23940 23950
DLVLENKCGK KAVYIKVRVI GSPNSPEGPL EYDDIQVRSV RVSWRPPADD
          23960 23970 23980 23990 24000
GGADILGYIL ERREVPKAAW YTIDSRVRGT SLVVKGLKEN VEYHFRVSAE
          24010 24020 24030 24040 24050
NQFGISKPLK SEEPVTPKTP LNPPEPPSNP PEVLDVTKSS VSLSWSRPKD
          24060 24070 24080 24090 24100
DGGSRVTGYY IERKETSTDK WVRHNKTQIT TTMYTVTGLV PDAEYQFRII
          24110 24120 24130 24140 24150
AQNDVGLSET SPASEPVVCK DPFDKPSQPG ELEILSISKD SVTLQWEKPE
          24160 24170 24180 24190 24200
CDGGKEILGY WVEYRQSGDS AWKKSNKERI KDKQFTIGGL LEATEYEFRV
          24210 24220 24230 24240 24250
FAENETGLSR PRRTAMSIKT KLTSGEAPGI RKEMKDVTTK LGEAAQLSCQ
          24260 24270 24280 24290 24300
IVGRPLPDIK WYRFGKELIQ SRKYKMSSDG RTHTLTVMTE EQEDEGVYTC
          24310 24320 24330 24340 24350
IATNEVGEVE TSSKLLLQAT PQFHPGYPLK EKYYGAVGST LRLHVMYIGR
          24360 24370 24380 24390 24400
PVPAMTWFHG QKLLQNSENI TIENTEHYTH LVMKNVQRKT HAGKYKVQLS
          24410 24420 24430 24440 24450
NVFGTVDAIL DVEIQDKPDK PTGPIVIEAL LKNSAVISWK PPADDGGSWI
          24460 24470 24480 24490 24500
TNYVVEKCEA KEGAEWQLVS SAISVTTCRI VNLTENAGYY FRVSAQNTFG
          24510 24520 24530 24540 24550
ISDPLEVSSV VIIKSPFEKP GAPGKPTITA VTKDSCVVAW KPPASDGGAK
          24560 24570 24580 24590 24600
IRNYYLEKRE KKQNKWISVT TEEIRETVFS VKNLIEGLEY EFRVKCENLG
          24610 24620 24630 24640 24650
GESEWSEISE PITPKSDVPI QAPHFKEELR NLNVRYQSNA TLVCKVTGHP
          24660 24670 24680 24690 24700
KPIVKWYRQG KEIIADGLKY RIQEFKGGYH QLIIASVTDD DATVYQVRAT
          24710 24720 24730 24740 24750
NQGGSVSGTA SLEVEVPAKI HLPKTLEGMG AVHALRGEVV SIKIPFSGKP
          24760 24770 24780 24790 24800
DPVITWQKGQ DLIDNNGHYQ VIVTRSFTSL VFPNGVERKD AGFYVVCAKN
          24810 24820 24830 24840 24850
RFGIDQKTVE LDVADVPDPP RGVKVSDVSR DSVNLTWTEP ASDGGSKITN
          24860 24870 24880 24890 24900
YIVEKCATTA ERWLRVGQAR ETRYTVINLF GKTSYQFRVI AENKFGLSKP
          24910 24920 24930 24940 24950
SEPSEPTITK EDKTRAMNYD EEVDETREVS MTKASHSSTK ELYEKYMIAE
          24960 24970 24980 24990 25000
DLGRGEFGIV HRCVETSSKK TYMAKFVKVK GTDQVLVKKE ISILNIARHR
          25010 25020 25030 25040 25050
NILHLHESFE SMEELVMIFE FISGLDIFER INTSAFELNE REIVSYVHQV
          25060 25070 25080 25090 25100
CEALQFLHSH NIGHFDIRPE NIIYQTRRSS TIKIIEFGQA RQLKPGDNFR
          25110 25120 25130 25140 25150
LLFTAPEYYA PEVHQHDVVS TATDMWSLGT LVYVLLSGIN PFLAETNQQI
          25160 25170 25180 25190 25200
IENIMNAEYT FDEEAFKEIS IEAMDFVDRL LVKERKSRMT ASEALQHPWL
          25210 25220 25230 25240 25250
KQKIERVSTK VIRTLKHRRY YHTLIKKDLN MVVSAARISC GGAIRSQKGV
          25260 25270 25280 25290 25300
SVAKVKVASI EIGPVSGQIM HAVGEEGGHV KYVCKIENYD QSTQVTWYFG
          25310 25320 25330 25340 25350
VRQLENSEKY EITYEDGVAI LYVKDITKLD DGTYRCKVVN DYGEDSSYAE
          25360 25370 25380 25390 25400
LFVKGVREVY DYYCRRTMKK IKRRTDTMRL LERPPEFTLP LYNKTAYVGE
          25410 25420 25430 25440 25450
NVRFGVTITV HPEPHVTWYK SGQKIKPGDN DKKYTFESDK GLYQLTINSV
          25460 25470 25480 25490 25500
TTDDDAEYTV VARNKYGEDS CKAKLTVTLH PPPTDSTLRP MFKRLLANAE
          25510 25520 25530 25540 25550
CQEGQSVCFE IRVSGIPPPT LKWEKDGQPL SLGPNIEIIH EGLDYYALHI
          25560 25570 25580 25590 25600
RDTLPEDTGY YRVTATNTAG STSCQAHLQV ERLRYKKQEF KSKEEHERHV
          25610 25620 25630 25640 25650
QKQIDKTLRM AEILSGTESV PLTQVAKEAL REAAVLYKPA VSTKTVKGEF
          25660 25670 25680 25690 25700
RLEIEEKKEE RKLRMPYDVP EPRKYKQTTI EEDQRIKQFV PMSDMKWYKK
          25710 25720 25730 25740 25750
IRDQYEMPGK LDRVVQKRPK RIRLSRWEQF YVMPLPRITD QYRPKWRIPK
          25760 25770 25780 25790 25800
LSQDDLEIVR PARRRTPSPD YDFYYRPRRR SLGDISDEEL LLPIDDYLAM
          25810 25820 25830 25840 25850
KRTEEERLRL EEELELGFSA SPPSRSPPHF ELSSLRYSSP QAHVKVEETR
          25860 25870 25880 25890 25900
KDFRYSTYHI PTKAEASTSY AELRERHAQA AYRQPKQRQR IMAEREDEEL
          25910 25920 25930 25940 25950
LRPVTTTQHL SEYKSELDEM SKEEKSRKKS RRQREVTEIT EIEEEYEISK
          25960 25970 25980 25990 26000
HAQRESSSSA SRLLRRRRSL SPTYIELMRP VSELIRSRPQ PAEEYEDDTE
          26010 26020 26030 26040 26050
RRSPTPERTR PRSPSPVSSE RSLSRFERSA RFDIFSRYES MKAALKTQKT
          26060 26070 26080 26090 26100
SERKYEVLSQ QPFTLDHAPR ITLRMRSHRV PCGQNTRFIL NVQSKPTAEV
          26110 26120 26130 26140 26150
KWYHNGVELQ ESSKIHYTNT SGVLTLEILD CHTDDSGTYR AVCTNYKGEA
          26160 26170 26180 26190 26200
SDYATLDVTG GDYTTYASQR RDEEVPRSVF PELTRTEAYA VSSFKKTSEM
          26210 26220 26230 26240 26250
EASSSVREVK SQMTETRESL SSYEHSASAE MKSAALEEKS LEEKSTTRKI
          26260 26270 26280 26290 26300
KTTLAARILT KPRSMTVYEG ESARFSCDTD GEPVPTVTWL RKGQVLSTSA
          26310 26320 26330 26340 26350
RHQVTTTKYK STFEISSVQA SDEGNYSVVV ENSEGKQEAE FTLTIQKARV
          26360 26370 26380 26390 26400
TEKAVTSPPR VKSPEPRVKS PEAVKSPKRV KSPEPSHPKA VSPTETKPTP
          26410 26420 26430 26440 26450
TEKVQHLPVS APPKITQFLK AEASKEIAKL TCVVESSVLR AKEVTWYKDG
          26460 26470 26480 26490 26500
KKLKENGHFQ FHYSADGTYE LKINNLTESD QGEYVCEISG EGGTSKTNLQ
          26510 26520 26530 26540 26550
FMGQAFKSIH EKVSKISETK KSDQKTTEST VTRKTEPKAP EPISSKPVIV
          26560 26570 26580 26590 26600
TGLQDTTVSS DSVAKFAVKA TGEPRPTAIW TKDGKAITQG GKYKLSEDKG
          26610 26620 26630 26640 26650
GFFLEIHKTD TSDSGLYTCT VKNSAGSVSS SCKLTIKAIK DTEAQKVSTQ
          26660 26670 26680 26690 26700
KTSEITPQKK AVVQEEISQK ALRSEEIKMS EAKSQEKLAL KEEASKVLIS
          26710 26720 26730 26740 26750
EEVKKSAATS LEKSIVHEEI TKTSQASEEV RTHAEIKAFS TQMSINEGQR
          26760 26770 26780 26790 26800
LVLKANIAGA TDVKWVLNGV ELTNSEEYRY GVSGSDQTLT IKQASHRDEG
          26810 26820 26830 26840 26850
ILTCISKTKE GIVKCQYDLT LSKELSDAPA FISQPRSQNI NEGQNVLFTC
          26860 26870 26880 26890 26900
EISGEPSPEI EWFKNNLPIS ISSNVSISRS RNVYSLEIRN ASVSDSGKYT
          26910 26920 26930 26940 26950
IKAKNFRGQC SATASLMVLP LVEEPSREVV LRTSGDTSLQ GSFSSQSVQM
          26960 26970 26980 26990 27000
SASKQEASFS SFSSSSASSM TEMKFASMSA QSMSSMQESF VEMSSSSFMG
          27010 27020 27030 27040 27050
ISNMTQLESS TSKMLKAGIR GIPPKIEALP SDISIDEGKV LTVACAFTGE
          27060 27070 27080 27090 27100
PTPEVTWSCG GRKIHSQEQG RFHIENTDDL TTLIIMDVQK QDGGLYTLSL
          27110
GNEFGSDSAT VNIHIRSI
```

Another example of a titin protein sequence is provided by UniProt as accession number Q8WZ42.

The N2-B splice variant of titin encodes the major N2-B cardiac muscle isoform, which lacks multiple exons in the region encoding PEVK repeats. This results in a shortened PEVK region in isoform N2-B compared to isoform IC. A sequence for such a human N2-B titin isoform is shown below (NCBI accession number NP 003310.4), provided below as SEQ ID NO:2.

| | |
|---|---|
| 1 | MTTQAPTFTQ PLQSVVVLEG STATFEAHIS GFPVPEVSWF |
| 41 | RDGQVISTST LPGVQISFSD GRAKLTIPAV TKANSGRYSL |
| 81 | KATNGSGQAT STAELLVKAE TAPPNFVQRL QSMTVRQGSQ |
| 121 | VRLQVRVTGI PTPVVKFYRD GAEIQSSLDF QISQEGDLYS |
| 161 | LLIAEAYPED SGTYSVNATN SVGRATSTAE LLVQGEEEVP |
| 201 | AKKTKTIVST AQISESRQTR IEKKIEAHFD ARSIATVEMV |
| 241 | IDGAAGQQLP HKTPPRIPPK PKSRSPTPPS IAAKAQLARQ |
| 281 | QSPSPIRHSP SPVRHVRAPT PSPVRSVSPA ARISTSPIRS |
| 321 | VRSPLLMRKT QASTVATGPE VPPPWKQEGY VASSSEAEMR |
| 361 | ETTLTTSTQI RTEERWEGRY GVQEQVTISG AAGAAASVSA |
| 401 | SASYAAEAVA TGAKEVKQDA DKSAAVATVV AAVDMARVRE |
| 441 | PVISAVEQTA QRTTTTAVHI QPAQEQVRKE AEKTAVTKVV |
| 481 | VAADKAKEQE LKSRTKEVIT TKQEQMHVTH EQIRKETEKT |
| 521 | FVPKVVISAA KAKEQETRIS EEITKKQKQV TQEAIMKETR |
| 561 | KTVVPKVIVA TPKVKEQDLV SRGREGITTK REQVQITQEK |
| 601 | MRKEAEKTAL STIAVATAKA KEQETILRTR ETMATRQEQI |
| 641 | QVTHGKVDVG KKAEAVATVV AAVDQARVRE PREPGHLEES |
| 681 | YAQQTTLEYG YKERISAAKV AEPPQRPASE PHVVPKAVKP |
| 721 | RVIQAPSETH IKTTDQKGMH ISSQIKKTTD LTTERLVHVD |
| 761 | KRPRTASPHF TVSKISVPKT EHGYEASIAG SAIATLQKEL |
| 781 | SATSSAQKIT KSVKAPTVKP SETRVRAEPT PLPQFPFADT |
| 841 | PDTYKSEAGV EVKKEVGVSI TGTTVREERF EVLHGREAKV |
| 881 | TETARVPAPV EIPVTPPTLVVSGLKNVTVIE GESVTLECHI |
| 921 | SGYPSPTVTW YREDYQIESS IDFQITFQSG IARLMIREAF |
| 961 | AEDSGRFTCS AVNEAGTVST SCYLAVQVSE EFEKETTAVT |
| 1001 | EKFTTEEKRF VESRDVVMTD TSLTEEQAGP GEPAAPYFIT |
| 1041 | KPVVQKLVEG GSVVFGCQVG GNPKPHVYWK KSGVPLTTGY |
| 1081 | RYKVSYNKQT GECKLVISMT FADDAGEYTI VVRNKHGETS |
| 1121 | ASASLLEEAD YELLMKSQQE MLYQTQVTAF VQEPKVGETA |
| 1161 | PGFVYSEYEK EYEKEQALIR KKMAKDTVVV RTYVEDQEFH |
| 1201 | ISSFEERLIK EIEYRIIKTT LEELLEEDGE EKMAVDISES |
| 1241 | EAVESGFDSR IKNYRILEGM GVTFHCKMSG YPLPKIAWYK |
| 1281 | DGKRIKHGER YQMDFLQDGR ASLRIPVVLP EDEGIYTAFA |
| 1321 | SNIKGNAICS GKLYVEPAAP LGAPTYIPTL EPVSRIRSLS |
| 1361 | PRSVSRSPIR MSPARMSPAR MSPARMSPAR MSPGRRLEET |
| 1401 | DESQLERLYK PVFVLKPVSF KCLEGQTARF DLKVVGRPMP |
| 1441 | ETFWFHDGQQ IVNDYTHKVV IKEDGTQSLI IVPATPSDSG |
| 1481 | EWTVVAQNRA GRSSISVILT VEAVEHQVKP MFVEKLKNVN |
| 1521 | IKEGSRLEMK VRATGNPNPD IVWLKNSDII VPHKYPKIRI |
| 1561 | EGTKGEAALK IDSTVSQDSA WYTATAINKA GRDTTRCKVN |
| 1601 | VEVEFAEPEP ERKLIIPRGT YRAKEIAAPE LEPLHLRYGQ |
| 1641 | EQWEEGDLYD KEKQQKPFFK KKLTSLRLKR FGPAHFECRL |
| 1681 | TPIGDPTMVV EWLHDGKPLE AANRLRMINE FGYCSLDYGV |
| 1721 | AYSRDSGIIT CRATNKYGTD HTSATLIVKD EKSLVEESQL |
| 1761 | PEGRKGLQRI EELERMAHEG ALTGVTTDQK EKQKPDIVLY |
| 1801 | PEPVRVLEGE TARFRCRVTG YPQPKVNWYL NGQLIRKSKR |
| 1841 | FRVRYDGIHY LDIVDCKSYD TGEVKVTAEN PEGVIEHKVK |
| 1881 | LEIQQREDFR SVLRRAPEPR PEFHVHEPGK LQFEVQKVDR |
| 1921 | PVDTTETKEV VKLKRAERIT HEKVPEESEE LRSKFKRRTE |
| 1961 | EGYYEAITAV ELKSRKKDES YEELLRKTKD ELLHWTKELT |
| 2001 | EEEKKALAEE GKITIPTFKP DKIELSPSME APKIFERIQS |
| 2041 | QTVGQGSDAH FRVRVVGKPD PECEWYKNGV KIERSDRIYW |
| 2081 | YWPEDNVCEL VIRDVTAEDS ASIMVKAINI AGETSSHAFL |
| 2121 | LVQAKQLITF TQELQDVVAK EKDTMATFEC ETSEPFVKVK |
| 2161 | WYKDGMEVHE GDKYRMHSDR KVHFLSILTI DTSDAEDYSC |
| 2201 | VLVEDENVKT TAKLIVEGAV VEFVKELQDI EVPESYSGEL |
| 2241 | ECIVSPENIE GKWYHNDVEL KSNGKYTITS RRGRQNLTVK |
| 2281 | DVTKEDQGEY SFVIDGKKTT CKLKMKPRPI AILQGLSDQK |
| 2321 | VCEGDIVQLE VKVSLESVEG VWMKDGQEVQ PSDRVHIVID |
| 2361 | KQSHMLLIED MTKEDAGNYS FTIPALGLST SGRVSVYSVD |
| 2401 | VITPLKDVNV IEGTKAVLEC KVSVPDVTSV KWYLNDEQIK |
| 2441 | PDDRVQAIVK GTKQRLVINR THASDEGPYK LIVGRVETNC |
| 2481 | NLSVEKIKII RGLRDLTCTE TQNVVFEVEL SHSGIDVLWN |
| 2521 | FKDKEIKPSS KYKIEAHGKI YKLTVLNMMK DDEGKYTFYA |
| 2561 | GENMTSGKLT VAGGAISKPL TDQTVAESQE AVFECEVANP |
| 2601 | DSKGEWLRDG KHLPLTNNIR SESDGHKRRL IIAATKLDDI |
| 2641 | GEYTYKVATS KTSAKLKVEA VKIKKTLKNL TVTETQDAVE |
| 2681 | TVELTHPNVK GVQWIKNGVV LESNEKYAIS VKGTIYSLRI |
| 2721 | KNCAIVDESV YGFRLGRLGA SARLHVETVK IIKKPKDVTA |
| 2761 | LENATVAFEV SVSHDTVPVK WFHKSVEIKP SDKHRLVSER |
| 2801 | KVHKLMLQNI SPSDAGEYTA VVGQLECKAK LFVETLHITK |
| 2841 | TMKNIEVPET KTASFECEVS HFNVPSMWLK NGVEIEMSEK |
| 2881 | FKIVVQGKLH QLIIMNTSTE DSAEYTFVCG NDQVSATLTV |
| 2921 | TPIMITSMLK DINAEEKDTI TFEVTVNYEG ISYKWLKNGV |
| 2961 | EIKSTDKCQM RTKKLTHSLN IRNVHFGDAA DYTFVAGKAT |
| 3001 | STATLYVEAR HIEFRKHIKD IKVLEKKRAM FECEVSEPDI |
| 3041 | TVQWMKDDQE LQITDRIKIQ KEKYVHRLLI PSTRMSDAGK |
| 3081 | YTVVAGGNVS TAKLFVEGRD VRIRSIKKEV QVIEKQRAVV |
| 3121 | EFEVNEDDVD AHWYKDGIEI NFQVQERHKY VVERRIHRMF |
| 3161 | ISETRQSDAG EYTFVAGRNR SSVTLYVNAP EPPQVLQELQ |
| 3201 | PVTVQSGKPA RFCAVISGRP QPKISWYKEE QLLSTGFKCK |
| 3241 | FLHDGQEYTL LLIEAFPEDA AVYTCEAKND YGVATTSASL |
| 3281 | SVEVPEVVSP DQEMPVYPPA IITPLQDTVT SEGQPARFQC |
| 3321 | RVSGTDLKVS WYSKDKKIKP SRFFRMTQFE DTYQLEIAEA |
| 3361 | YPEDEGTYTF VASNAVGQVS STANLSLEAP ESILHERIEQ |
| 3401 | EIEMEMKEFS SSFLSAEEEG LHSAELQLSK INETLELLSE |
| 3441 | SPVYPTKFDS EKEGTGPIFI KEVSNADISM GDVATLSVTV |
| 3481 | IGIPKPKIQW FFNGVLLTPS ADYKFVFDGD DHSLIILFTK |
| 3521 | LEDEGEYTCM ASNDYGKTIC SAYLKINSKG EGHKDTETES |
| 3561 | AVAKSLEKLG GPCPPHFLKE LKPIRCAQGL PAIFEYTVVG |
| 3601 | EPAPTVTWFK ENKQLCTSVY YTIIHNPNGS GTFIVNDPQR |
| 3641 | EDSGLYICKA ENMLGESTCA AELLVLLEDT DMTDTPCKAK |
| 3681 | STPEAPEDFP QTPLKGPAVE ALDSEQEIAT FVKDTILKAA |
| 3721 | LITEENQQLS YEHIAKANEL SSQLPLGAQE LQSILEQDKL |
| 3761 | TPESTREFLC INGSIHFQPL KEPSPNLQLQ IVQSQKTFSK |
| 3801 | EGILMPEEPE TQAVLSDTEK IFPSAMSIEQ INSLTVEPLK |
| 3841 | TLLAEPEGNY PQSSIEPPMH SYLTSVAEEV LSPKEKTVSD |
| 3881 | TNREQRVTLQ KQEAQSALIL SQSLAEGHVE SLQSPDVMIS |
| 3921 | QVNYEPLVPS EHSCTEGGKI LIESANPLEN AGQDSAVRIE |
| 3961 | EGKSLRFPLA LEEKQVLLKE EHSDNVVMPP DQIIESKREP |
| 4001 | VAIKKVQEVQ GRDLLSKESL LSGIPEEQRL NLKIQICRAL |
| 4041 | QAAVASEQPG LFSEWLRNIE KVEVEAVNIT QEPRHIMCMY |
| 4081 | LVTSAKSVTE EVTIIIEDVD PQMANLKMEL RDALCAIIYE |
| 4121 | EIDILTAEGP RIQQGAKTSL QEEMDSFSGS QKVEPITEPE |
| 4161 | VESKYLISTE EVSYFNVQSR VKYLDATPVT KGVASAVVSD |
| 4201 | EKQDESLKPS EEKEESSSES GTEEVATVKI QEAEGGLIKE |
| 4241 | DGPMIHTPLV DTVSEEGDIV HLTTSITNAK EVNWYFENKL |
| 4281 | VPSDEKFKCL QDQNTYTLVI DKVNTEDHQG EYVCEALNDS |
| 4321 | GKTATSAKLT VVKRAAPVIK RKIEPLEVAL GHLAKFTCEI |
| 4361 | QSAPNVRFQW FKAGREIYES DKCSIRSSKY ISSLEILRTQ |
| 4401 | VVDCGEYTCK ASNEYGSVSC TATLTVTVPG GEKKVRKLLP |
| 4441 | ERKPEPKEEV VLKSVLRKRP EEEEPKVEPK KLEKVKKPAV |
| 4481 | PEPPPPKPVE EVEVPTVTKR ERKIPEPTKV PEIKPAIPLP |
| 4521 | APEPKPKPEA EVKTIKPPPV EPEPTPIAAP VTVPVVGKKA |
| 4561 | EAKAPKEEAA KPKGPIKGVP KKTPSPIEAE RRKLRPGSGG |
| 4601 | EKPPDEAPFT YQLKAVPLKF VKEIKDIILT ESEFVGSSAI |
| 4641 | FECLVSPSTA ITTWMKDGSN IRESPKHRFI ADGKDRKLHI |
| 4681 | IDVQLSDAGE YTCVLRLGNK EKTSTAKLVV EELPVRFVKT |
| 4721 | LEEEVTVVKG QPLYLSCELN KERDVVWRKD GKIVVEKPGR |
| 4761 | IVPGVIGLMR ALTINDADDT DAGTYTVTVE NANNLECSSC |
| 4801 | VKVVEVIRDW LVKPIRDQHV KPKGTAIFAC DIAKDTPNIK |
| 4841 | WFKGYDEIPA EPNDKTEILR DGNHLYLKIK NAMPEDIAEY |
| 4881 | AVEIEGKRYP AKLTLGEREV ELLKPIEDVT IYEKESASFD |
| 4921 | AEISEADIPG QWKLKGELLR PSPTCEIKAE GGKRFLTLHK |
| 4961 | VKLDQAGEVL YQALNAITTA ILTVKEIELD FAVPLKDVTV |
| 5001 | PERRQARFEC VLTREANVIW SKGPDIIKSS DKFDIIADGK |
| 5041 | KHILVINDSQ FDDEGVYTAE VEGKKTSARL FVTGIRLKFM |
| 5081 | SPLEDQTVKE GETATFVCEL SHEKMHVVWF KNDAKLHTSR |
| 5121 | TVLISSEGKT HKLEMKEVTL DDISQIKAQV KELSSTAQLK |
| 5161 | VLEADPYFTV KLHDKTAVEK DEITLKCEVS KDVPVKWFKD |
| 5201 | GEEIVPSPKY SIKADGLRRI LKIKKADLKD KGEYVCDCGT |
| 5241 | DKTKANVTVE ARLIKVEKPL YGVEVFVGET AHFEIELSEP |
| 5281 | DVHGQWKLKG QPLTASPDCE IIEDGKKHIL ILHNCQLGMT |
| 5321 | GEVSFQAANA KSAANLKVKE LPLIFITPLS DVKVFEKDEA |
| 5361 | KFECEVSREP KTFRWLKGTQ EITGDDRFEL IKDGTKHSMV |
| 5401 | IKSAAFEDEA KYMFEAEDKH TSGKLIIEGI RLKFLTPLKD |
| 5441 | VTAKEKESAV FTVELSHDNI RVKWFKNDQR LHTTRSVSMQ |
| 5481 | DEGKTHSITF KDLSIDDTSQ IRVEAMGMSS EAKLTVLEGD |
| 5521 | PYFTGKLQDY TGVEKDEVIL QCEISKADAP VKWFKDGKEI |
| 5561 | KPSKNAVIKA DGKKRMLILK KALKSDIGQY TCDCGTDKTS |
| 5601 | GKLDIEDREI KLVRPLHSVE VMETETARFE TEISEDDIHA |
| 5641 | NWKLKGEALL QTPDCEIKEE GKIHSLVLHN CRLDQTGGVD |
| 5681 | FQAANVKSSA HLRVKPRVIG LLRPLKDVTV TAGETATFDC |
| 5721 | ELSYEDIPVE WYLKGKKLEP SDKVVPRSEG KVHTLTLRDV |
| 5761 | KLEDAGEVQL TAKDFKTHAN LFVKEPPVEF TKPLEDQTVE |
| 5801 | EGATAVLECE VSRENAKVKW FKNGTEILKS KKYEIVADGR |
| 5841 | VRKLVIHDCT PEDIKTYTCD AKDFKTSCNL NVVPPHVEFL |
| 5881 | RPLTDLQVRE KEMARFECEL SRENAKVKWF KDGAEIKKGK |
| 5921 | KYDIISKGAV RILVINKCLL DDEAEYSCEV RTARTSGMLT |
| 5961 | VLEEEAVFTK NLANIEVSET DTIKLVCEVS KPGAEVIWYK |
| 6001 | GDEEIIETGR YEILTEGRKR ILVIQNAHLE DAGNYNCRLP |
| 6041 | SSRTDGKVKV HELAAEFISK PQNLEILEGE KAEFVCSISK |
| 6081 | ESFPVQWKRD DKTLESGDKY DVIADGKKRV LVVKDATLQD |
| 6121 | MGTYVVMVGA ARAAAHLTVI EKLRIVVPLK DTRVKEQQEV |
| 6161 | VFNCEVNTEG AKAKWFRNEE AIFDSSKYII LQKDLVYTLR |
| 6201 | IRDAHLDDQA NYNVSLINHR GENVKSAANL IVEEEDLRIV |
| 6241 | EPLKDIETME KKSVTFWCKV NRLNVTLKWT KNGEEVPFDN |
| 6281 | RVSYRVDKYK HMLTIKDCGF PDEGEYIVTA GQDKSVAELL |
| 6321 | IIEAPTEFVE HLEDQTVTEF DDAVFSCQLS REKANVKWYR |
| 6361 | NGREIKEGKK YKFEKDGSIH RLIIKDCRLD DECEYACGVE |
| 6401 | DRKSRARLFV EEIPVEIIRP PQDILEAPGA DVVFLAELNK |
| 6441 | DKVEVQWLRN NMVVVQGDKH QMMSEGKIHR LQICDIKPRD |
| 6481 | QGEYRFIAKD KEARAKLELA AAPKIKTADQ DLVVDVGKPL |
| 6521 | TMVVPYDAYP KAEAEWFKEN EPLSTKTIDT TAEQTSFRIL |
| 6561 | EAKKGDKGRY KIVLQNKHGK AEGFINLKVI DVPGPVRNLE |
| 6601 | VTETFDGEVS LAWEEPLTDG GSKIIGYVVE RRDIKRKTWV |
| 6641 | LATDRAESCE FTVTGLQKGG VEYLFRVSAR NRVGTGEPVE |
| 6681 | TDNPVEARSK YDVPGPPLNV TITDVNRFGV SLTWEPPEYD |
| 6721 | GGAEITNYVI ELRDKTSIRW DTAMTVRAED LSATVTDVVE |
| 6761 | GQEYSFRVRA QNRIGVGKPS AATPFVKVAD PIERPSPPVN |
| 6801 | LTSSDQTQSS VQLKWEPPLK DGGSPILGYI IERCEEGKDN |
| 6841 | WIRCNMKLVP ELTYKVTGLE KGNKYLYRVS AENKAGVSDP |
| 6881 | SEILGPLTAD DAFVEPTMDL SAFKDGLEVI VPNPITILVP |
| 6921 | STGYPRPTAT WCFGDKVLET GDRVKMKTLS AYAELVISPS |
| 6961 | ERSDKGIYTL KLENRVKTIS GEIDVNVIAR PSAPKELKFG |
| 7001 | DITKDSVHLT WEPPDDDGGS PLTGYVVEKR EVSRKTWTKV |
| 7041 | MDFVTDLEFT VPDLVQGKEY LFKVCARNKC GPGEPAYVDE |
| 7081 | PVNMSTPATV PDPPENVKWR DRTANSIFLT WDPPKNDGGS |
| 7121 | RIKGYIVERC PRGSDKWVAC GEPVAETKME VTGLEEGKWY |
| 7161 | AYRVKALNRQ GASKPSRPTE EIQAVDTQEA PEIFLDVKLL |
| 7201 | AGLTVKAGTK IELPATVTGK PEPKITWTKA DMILKQDKRI |
| 7241 | TIENVPKKST VTIVDSKRSD TGTYIIEAVN VCGRATAVVE |
| 7281 | VNVLDKPGPP AAFDITDVTN ESCLLTWNPP RDDGGSKITN |
| 7321 | YVVERRATDS EVWHKLSSTV KDTNFKATKL IPNKEYIFRV |
| 7361 | AAENMYGVGE PVQASPITAK YQFDPPGPPT RLEPSDITKD |
| 7401 | AVTLTWCEPD DDGGSPITGY WVERLDPDTD KWVRCNKMPV |
| 7441 | KDTTYRVKGL TNKKKYRFRV LAENLAGPGK PSKSTEPILI |
| 7481 | KDPIDPPWPP GKPTVKDVGK TSVRLNWTKP EHDGGAKIES |
| 7521 | YVIEMLKTGT DEWVRVAEGV PTTQHLLPGL MEGQEYSFRV |
| 7561 | RAVNKAGESE PSEPSDPVLC REKLYPPSPP RWLEVINITK |
| 7601 | NTADLKWTVP EKDGGSPITN YIVEKRDVRR KGWQTVDTTV |
| 7641 | KDTKCTVTPL TEGSLYVFRV AAENAIGQSD YTEIEDSVLA |
| 7681 | KDTFTTPGPP YALAVVDVTK RHVDLKWEPP KNDGGRPIQR |
| 7721 | YVIEKKERLG TRWVKAGKTA GPDCNFRVTD VIEGTEVQFQ |
| 7761 | VRAENEAGVG HPSEPTEILS IEDPTSPPSP PLDLHVTDAG |
| 7801 | RKHIAIAWKP PEKNGGSPII GYHVEMCPVG TEKWMRVNSR |
| 7841 | PIKDLKFKVE EGVVPDKEYV LRVRAVNAIG VSEPSEISEN |
| 7881 | VVAKDPDCKP TIDLETHDII VIEGEKLSIP VPFRAVPVPT |
| 7921 | VSWHKDGKEV KASDRLTMKN DHISAHLEVP KSVRADAGIY |
| 7961 | TITLENKLGS ATASINVKVI GLPGPCKDIK ASDITKSSCK |
| 8001 | LTWEPPEFDG GTPILHYVLE RREAGRRTYI PVMSGENKLS |
| 8041 | WTVKDLIPNG EYFFRVKAVN KVGGGEYIEL KNPVIAQDPK |
| 8081 | QPPDPPVDVE VHNPTAEAMT ITWKPPLYDG GSKIMGYIIE |
| 8121 | KIAKGEERWK RCNEHLVPIL TYTAKGLEEG KEYQFRVRAE |
| 8161 | NAAGISEPSR ATPPTKAVDP IDAPKVILRT SLEVKRGDEI |
| 8201 | ALDASISGSP YPTITWIKDE NVIVPEEIKK RAAPLVRRRK |
| 8241 | GEVQEEEPFV LPLTQRLSID NSKKGESQLR VRDSLRPDHG |
| 8281 | LYMIKVENDH GIAKAPCTVS VLDTPGPPIN FVFEDIRKTS |
| 8321 | VLCKWEPPLD DGGSEIINYT LEKKDKTKPD SEWIVVTSTL |
| 8361 | RHCKYSVTKL IEGKEYLFRV RAENRFGPGP PCVSKPLVAK |
| 8401 | DPFGPPDAPD KPIVEDVTSN SMLVKWNEPK DNGSPILGYW |
| 8441 | LEKREVNSTH WSRVNKSLLN ALKANVDGLL EGLTYVFRVC |
| 8481 | AENAAGPGKF SPPSDPKTAH DPISPPGPPI PRVTDTSSTT |
| 8521 | IELEWEPPAF NGGGEIVGYF VDKQLVGTNE WSRCTEKMIK |
| 8561 | VRQYTVKEIR EGADYKLRVS AVNAAGEGPP GETQPVTVAE |
| 8601 | PQEPPAVELD VSVKGGIQIM AGKTLRIPAV VTGRPVPTKV |
| 8641 | WTKEEGELDK DRVVIDNVGT KSELIIKDAL RKDHGRYVIT |
| 8681 | ATNSCGSKFA AARVEVFDVP GPVLDLKPVV TNRKMCLLNW |
| 8721 | SDPEDDGGSE ITGFIIERKD AKMHTWRQPI ETERSKCDIT |
| 8761 | GLLEGQEYKF RVIAKNKFGC GPPVEIGPIL AVDPLGPPTS |
| 8801 | PERLTYTERT KSTITLDWKE PRSNGGSPIQ GYIIEKRRHD |
| 8841 | KPDFERVNKR LCPTTSFLVE NLDEHQMYEF RVKAVNEIGE |
| 8881 | SEPSLPLNVV IQDDEVPPTI KLRLSVRGDT IKVKAGEPVH |
| 8921 | IPADVTGLPM PKIEWSKNET VIEKPTDALQ ITKEEVSRSE |
| 8961 | AKTELSIPKA VREDKGTYTV TASNRLGSVF RNVHVEVYDR |
| 9001 | PSPPRNLAVT DIKAESCYLT WDAPLDNGGS EITHYVIDKR |
| 9041 | DASRKKAEWE EVTNTAVEKR YGIWKLIPNG QYEFRVRAVN |
| 9081 | KYGISDECKS DKVVIQDPYR LPGPPGKPKV LARTKGSMLV |
| 9121 | SWTPPLDNGG SPITGYWLEK REEGSPYWSR VSRAPITKVG |
| 9161 | LKGVEFNVPR LLEGVKYQFR AMAINAAGIG PPSEPSDPEV |
| 9201 | AGDPIFPPGP PSCPEVKDKT KSSISLGWKP PAKDGGSPIK |
| 9241 | GYIVEMQEEG TTDWKRVNEP DKLITTCECV VPNLKELRKY |
| 9281 | RFRVKAVNEA GESEPSDTTG EIPATDIQEE PEVFIDIGAQ |
| 9321 | DCLVCKAGSQ IRIPAVIKGR PTPKSSWEFD GKAKKAMKDG |
| 9361 | VHDIPEDAQL ETAENSSVII IPECKRSHTG KYSITAKNKA |
| 9401 | GQKTANCRVK VMDVPGPPKD LKVSDITRGS CRLSWKMPDD |
| 9441 | DGGDRIKGYV IEKRTIDGKA WTKVNPDCGS TTFVVPDLLS |
| 9481 | EQQYFFRVRA ENRFGIGPPV ETIQRTTARD PIYPPDPPIK |
| 9521 | LKIGLITKNT VHLSWKPPKN DGGSPVTHYI VECLAWDPTG |
| 9561 | TKKEAWRQCN KRDVEELQFT VEDLVEGGEY EFRVKAVNAA |
| 9601 | GVSKPSATVG PVTVKDQTCP PSIDLKEFME VEEGTNVNIV |
| 9641 | AKIKGVPFPT LTWFKAPPKK PDNKEPVLYD THVNKLVVDD |
| 9681 | TCTLVIPQSR RSDTGLYTIT AVNNLGTASK EMRLNVLGRP |
| 9721 | GPPVGPIKFE SVSADQMTLS WFPPKDDGGS KITNYVIEKR |
| 9761 | EANRKTWVHV SSEPKECTYT IPKLLEGHEY VFRIMAQNKY |
| 9801 | GIGEPLDSEP ETARNLFSVP GAPDKPTVSS VTRNSMTVNW |
| 9841 | EEPEYDGGSP VTGYWLEMKD TTSKRWKRVN RDPIKAMTLG |
| 9881 | VSYKVTGLIE GSDYQFRVYA INAAGVGPAS LPSDPATARD |
| 9921 | PIAPPGPPFP KVTDWTKSSA DLEWSPPLKD GGSKVTGYIV |
| 9961 | EYKEEGKEEW EKGKDKEVRG TKLVVTGLKE GAFYKFRVRA |
| 10001 | VNIAGIGEPG EVTDVIEMKD RLVSPDLQLD ASVRDRIVVH |
| 10041 | AGGVIRIIAY VSGKPPPTVT WNMNERTLPQ EATIETTAIS |
| 10081 | SSMVIKNCQR SHQGVYSLLA KNEAGERKKT IIVDVLDVPG |
| 10121 | PVGTPFLAHN LTNESCKLTW FSPEDDGGSP ITNYVIEKRE |
| 10161 | SDRRAWTPVT YTVTRQNATV QGLIQGKAYF FRIAAENSIG |
| 10201 | MGPFVETSEA LVIREPITVP ERPEDLEVKE VTKNTVTLTW |
| 10241 | NPPKYDGGSE IINYVLESRL IGTEKFHKVT NDNLLSRKYT |
| 10281 | VKGLKEGDTY EYRVSAVNIV GQGKPSFCTK PITCKDELAP |
| 10321 | PTLHLDERDK LTIRVGEAFA LTGRYSGKPK PKVSWFKDEA |
| 10361 | DVLEDDRTHI KTTPATLALE KIKAKRSDSG KYCVVVENST |
| 10401 | GSRKGFCQVN VVDRPGPPVG PVSFDEVTKD YMVISWKPPL |
| 10441 | DDGGSKITNY IIEKKEVGKD VWMPVTSASA KTTCKVSKLL |
| 10481 | EGKDYIFRIH AENLYGISDP LVSDSMKAKD RFRVPDAPDQ |
| 10521 | PIVTEVTKDS ALVTWNKPHD GGKPITNYIL EKRETMSKRW |
| 10561 | ARVTKDPIHP YTKFRVPDLL EGCQYEFRVS AENEIGIGDP |
| 10601 | SPPSKPVFAK DPIAKPSPPV NPEAIDTTCN SVDLTWQPPR |
| 10641 | HDGGSKILGY IVEYQKVGDE EWRRANHTPE SCPETKYKVT |
| 10681 | GLRDGQTYKF RVLAVNAAGE SDPAHVPEPV LVKDRLEPPE |
| 10721 | LILDANMARE QHIKVGDTLR LSAIIKGVPF PKVTWKKEDR |
| 10761 | DAPTKARIDV TPVGSKLEIR NAAHEDGGIY SLTVENPAGS |
| 10801 | KTVSVKVLVL DKPGPPRDLE VSEIRKDSCY LTWKEPLDDG |
| 10841 | GSVITNYVVE RRDVASAQWS PLSATSKKKS HFAKHLNEGN |
| 10881 | QYLFRVAAEN QYGRGPFVET PKPIKALDPL HPPGPPKDLH |
| 10921 | HVDVDKTEVS LVWNKPDRDG GSPITGYLVE YQEEGTQDWI |
| 10961 | KFKTVTNLEC VVTGLQQGKT YRFRVKAENI VGLGLPDTTI |
| 11001 | PIECQEKLVP PSVELDVKLI EGLVVKAGTT VRFPAIIRGV |
| 11041 | PVPTAKWTTD GSEIKTDEHY TVETDNFSSV LTIKNCLRRD |
| 11081 | TGEYQITVSN AAGSKTVAVH LTVLDVPGPP TGPINILDVT |
| 11121 | PEHMTISWQP PKDDGGSPVI NYIVEKQDTR KDTWGVVSSG |
| 11161 | SSKTKLKIPH LQKGCEYVFR VRAENKIGVG PPLDSTPTVA |
| 11201 | KHKFSPPSPP GKPVVTDITE NAATVSWTLP KSDGGSPITG |
| 11241 | YYMERREVTG KWVRVNKTPI ADLKFRVTGL YEGNTYEFRV |
| 11281 | FAENLAGLSK PSPSSDPIKA CRPIKPPGPP INPKLKDKSR |
| 11321 | ETADLVWTKP LSDGGSPILG YVVECQKPGT AQWNRINKDE |
| 11361 | LIRQCAFRVP GLIEGNEYRF RIKAANIVGE GEPRELAESV |
| 11401 | IAKDILHPPE VELDVTCRDV ITVRVGQTIR ILARVKGRPE |
| 11441 | PDITWTKEGK VLVREKRVDL IQDLPRVELQ IKEAVRADHG |
| 11481 | KYIISAKNSS GHAQGSAIVN VLDRPGPCQN LKVTNVTKEN |
| 11521 | CTISWENPLD NGGSEITNFI VEYRKPNQKG WSIVASDVTK |
| 11561 | RLIKANLLAN NEYYFRVCAE NKVGVGPTIE TKTPILAINP |
| 11601 | IDRPGEPENL HIADKGKTFV YLKWRRPDYD GGSPNLSYHV |
| 11641 | ERRLKGSDDW ERVHKGSIKE THYMVDRCVE NQIYEFRVQT |
| 11681 | KNEGGESDWV KTEEVVVKED LQKPVLDLKL SGVLTVKAGD |
| 11721 | TIRLEAGVRG KPFPEVAWTK DKDATDLTRS PRVKIDTRAD |
| 11761 | SSKFSLTKAK RSDGGKYVVT ATNTAGSFVA YATVNVLDKP |
| 11801 | GPVRNLKIVD VSSDRCTVCW DPPEDDGGCE IQNYILEKCE |
| 11841 | TKRMVWSTYS ATVLTPGTTV TRLIEGNEYI FRVRAENKIG |
| 11881 | TGPPTESKPV IAKTKYDKPG RPDPPEVTKV SKEEMTVVWN |
| 11921 | PPEYDGGKSI TGYFLEKKEK HSTRWVPVNK SAIPERRMKV |
| 11961 | QNLLPDHEYQ FRVKAENEIG IGEPSLPSRP VVAKDPIEPP |
| 12001 | GPPTNFRVVD TTKHSITLGW GKPVYDGGAP IIGYVVEMRP |
| 12041 | KIADASPDEG WKRCNAAAQL VRKEFTVTSL DENQEYEFRV |
| 12081 | CAQNQVGIGR PAELKEAIKP KEILEPPEID LDASMRKLVI |
| 12121 | VRAGCPIRLF AIVRGRPAPK VTWRKVGIDN VVRKGQVDLV |
| 12161 | DTMAFLVIPN STRDDSGKYS LTLVNPAGEK AVFVNVRVLD |
| 12201 | TPGPVSDLKV SDVTKTSCHV SWAPPENDGG SQVTHYIVEK |
| 12241 | READRKTWST VTPEVKKTSF HVTNLVPGNE YYFRVTAVNE |
| 12281 | YGPGVPTDVP KPVLASDPLS EPDPPRKLEV TEMTKNSATL |
| 12321 | AWLPPLRDGG AKIDGYITSY REEEQPADRW TEYSVVKDLS |
| 12361 | LVVTGLKEGK KYKFRVAARN AVGVSLPREA EGVYEAKEQL |
| 12401 | LPPKILMPEQ ITIKAGKKLR IEAHVYGKPH PTCKWKKGED |
| 12441 | EVVTSSHLAV HKADSSSILI IKDVTRKDSG YYSLTAENSS |
| 12481 | GTDTQKIKVV VMDAPGPPQP PFDISDIDAD ACSLSWHIPL |
| 12521 | EDGGSNITNY IVEKCDVSRG DWVTALASVT KTSCRVGKLI |
| 12561 | PGQEYIFRVR AENRFGISEP LTSPKMVAQF PFGVPSEPKN |
| 12601 | ARVTKVNKDC IFVAWDRPDS DGGSPIIGYL IERKERNSLL |
| 12641 | WVKANDTLVR STEYPCAGLV EGLEYSFRIY ALNKAGSSPP |
| 12681 | SKPTEYVTAR MPVDPPGKPE VIDVTKSTVS LIWARPKHDG |
| 12721 | GSKIIGYFVE ACKLPGDKWV RCNTAPHQIP QEEYTATGLE |
| 12761 | EKAQYQFRAI ARTAVNISPP SEPSDPVTIL AENVPPRIDL |
| 12801 | SVAMKSLLTV KAGTNVCLDA TVFGKPMPTV SWKKDGTLLK |
| 12841 | PAEGIKMAMQ RNLCTLELFS VNRKDSGDYT ITAENSSGSK |
| 12881 | SATIKLKVLD KPGPPASVKI NKMYSDRAML SWEPPLEDGG |
| 12921 | SEITNYIVDK RETSRPNWAQ VSATVPITSC SVEKLIEGHE |
| 12961 | YQFRICAENK YGVGDPVFTE PAIAKNPYDP PGRCDPPVIS |
| 13001 | NITKDHMTVS WKPPADDGGS PITGYLLEKR ETQAVNWTKV |
| 13041 | NRKPIIERTL KATGLQEGTE YEFRVTAINK AGPGKPSDAS |
| 13081 | KAAYARDPQY PPGPPAFPKV YDTTRSSVSL SWGKPAYDGG |
| 13121 | SPIIGYLVEV KRADSDNWVR CNLPQNLQKT RFEVTGLMED |
| 13161 | TQYQFRVYAV NKIGYSDPSD VPDKHYPKDI LIPPEGELDA |
| 13201 | DLRKTLILRA GVTMRLYVPV KGRPPPKITW SKPNVNLRDR |
| 13241 | IGLDIKSTDF DTFLRCENVN KYDAGKYILT LENSCGKKEY |
| 13281 | TIVVKVLDTP GPPVNVTVKE ISKDSAYVTW EPPIIDGGSP |
| 13321 | IINYVVQKRD AERKSWSTVT TECSKTSFRV ANLEEGKSYF |
| 13361 | FRVFAENEYG IGDPGETRDA VKASQTPGPV VDLKVRSVSK |
| 13401 | SSCSIGWKKP HSDGGSRIIG YVVDFLTEEN KWQRVMKSLS |
| 13441 | LQYSAKDLTE GKEYTFRVSA ENENGEGTPS EITVVARDDV |
| 13481 | VAPDLDLKGL PDLCYLAKEN SNFRLKIPIK GKPAPSVSWK |
| 13521 | KGEDPLATDT RVSVESSAVN TTLIVYDCQK SDAGKYTITL |
| 13561 | KNVAGTKEGT ISIKVVGKPG IPTGPIKFDE VTAEAMTLKW |
| 13601 | APPKDDGGSE ITNYILEKRD SVNNKWVTCA SAVQKTTFRV |
| 13641 | TRLHEGMEYT FRVSAENKYG VGEGLKSEPI VARHPFDVPD |
| 13681 | APPPPNIVDV RHDSVSLTWT DPKKTGGSPI TGYHLEFKER |
| 13721 | NSLLWKRANK TPIRMRDFKV TGLTEGLEYE FRVMAINLAG |
| 13761 | VGKPSLPSEP VVALDPIDPP GKPEVINITR NSVTLIWTEP |
| 13801 | KYDGGHKLTG YIVEKRDLPS KSWMKANHVN VPECAFTVTD |
| 13841 | LVEGGKYEFR IRAKNTAGAI SAPSESTETI ICKDEYEAPT |
| 13881 | IVLDPTIKDG LTIKAGDTIV LNAISILGKP LPKSSWSKAG |
| 13921 | KDIRPSDITQ ITSTPTSSML TIKYATRKDA GEYTITATNP |
| 13961 | FGTKVEHVKV TVLDVPGPPG PVEISNVSAE KATLTWTPPL |
| 14001 | EDGGSPIKSY ILEKRETSRL LWTVVSEDIQ SCRHVATKLI |
| 14041 | QGNEYIFRVS AVNHYGKGEP VQSEPVKMVD RFGPPGPPEK |
| 14081 | PEVSNVTKNT ATVSWKRPVD DGGSEITGYH VERREKKSLR |
| 14121 | WVRAIKTPVS DLRCKVTGLQ EGSTYEFRVS AENRAGIGPP |
| 14161 | SEASDSVLMK DAAYPPGPPS NPHVTDTTKK SASLAWGKPH |
| 14201 | YDGGLEITGY VVEHQKVGDE AWIKDTTGTA LRITQFVVPD |
| 14241 | LQTKEKYNFR ISAINDAGVG EPAVIPDVEI VEREMAPDFE |
| 14281 | LDAELRRTLV VRAGLSIRIF VPIKGRPAPE VTWTKDNINL |
| 14321 | KNRANIENTE SFTLLIIPEC NRYDTGKFVM TIENPAGKKS |
| 14361 | GFVNVRVLDT PGPVLNLRPT DITKDSVTLH WDLPLIDGGS |
| 14401 | RITNYIVEKR EATRKSYSTA TTKCHKCTYK VTGLSEGCEY |
| 14441 | FFRVMAENEY GIGEPTETTE PVKASEAPSP PDSLNIMDIT |
| 14481 | KSTVSLAWPK PKHDGGSKIT GYVIEAQRKG SDQWTHITTV |
| 14521 | KGLECVVRNL TEGEEYTFQV MAVNSAGRSA PRESRPVIVK |
| 14561 | EQTMLPELDL RGIYQKLVIA KAGDNIKVEI PVLGRPKPTV |
| 14601 | TWKKGDQILK QTQRVNFETT ATSTILNINE CVRSDSGPYP |
| 14641 | LTARNIVGEV GDVITIQVHD IPGPPTGPIK FDEVSSDFVT |
| 14681 | FSWDPPENDG GVPISNYVVE MRQTDSTTWV ELATTVIRTT |
| 14721 | YKATRLTTGL EYQFRVKAQN RYGVGPGITS ACIVANYPFK |
| 14761 | VPGPPGTPQV TAVTKDSMTI SWHEPLSDGG SPILGYHVER |
| 14801 | KERNGILWQT VSKALVPGNI FKSSGLTDGI AYEFRVIAEN |
| 14841 | MAGKSKPSKP SEPMLALDPI DPPGKPVPLN ITRHTVTLKW |
| 14881 | AKPEYTGGFK ITSYIVEKRD LPNGRWLKAN FSNILENEFT |
| 14921 | VSGLTEDAAY EFRVIAKNAA GAISPPSEPS DAITCRDDVE |
| 14961 | APKIKVDVKF KDTVILKAGE AFRLEADVSG RPPPTMEWSK |
| 15001 | DGKELEGTAK LEIKIADFST NLVNKDSTRR DSGAYTLTAT |
| 15041 | NPGGFAKHIF NVKVLDRPGP PEGPLAVTEV TSEKCVLSWF |
| 15081 | PPLDDGGAKI DHYIVQKRET SRLAWTNVAS EVQVTKLKVT |
| 15121 | KLLKGNEYIF RVMAVNKYGV GEPLESEPVL AVNPYGPPDP |
| 15161 | PKNPEVTTIT KDSMVVCWGH PDSDGGSEII NYIVERRDKA |
| 15201 | GQRWIKCNKK TLTDLRYKVS GLTEGHEYEF RIMAENAAGI |
| 15241 | SAPSPTSPFY KACDTVFKPG PPGNPRVLDT SRSSISIAWN |
| 15281 | KPIYDGGSEI TGYMVEIALP EEDEWQIVTP PAGLKATSYT |
| 15321 | ITGLTENQEY KIRIYAMNSE GLGEPALVPG TPKAEDRMLP |
| 15361 | PEIELDADLR KVVTIRACCT LRLFVPIKGR PAPEVKWARD |
| 15401 | HGESLDKASI ESTSSYTLLI VGNVNRFDSG KYILTVENSS |
| 15441 | GSKSAFVNVR VLDTPGPPQD LKVKEVTKTS VTLTWDPPLL |
| 15481 | DGGSKIKNYI VEKRESTRKA YSTVATNCHK TSWKVDQLQE |
| 15521 | GCSYYFRVLA ENEYGIGLPA ETAESVKASE RPLPPGKITL |
| 15561 | MDVTRNSVSL SWEKPEHDGG SRILGYIVEM QTKGSDKWAT |
| 15601 | CATVKVTEAT ITGLIQGEEY SFRVSAQNEK GISDPRQLSV |
| 15641 | PVIAKDLVIP PAFKLLFNTF TVLAGEDLKV DVPFIGRPTP |
| 15681 | AVTWHKDNVP LKQTTRVNAE STENNSLLTI KDACREDVGH |
| 15721 | YVVKLTNSAG EAIETLNVIV LDKPGPPTGP VKMDEVTADS |
| 15761 | ITLSWGPPKY DGGSSINNYI VEKRDTSTTT WQIVSATVAR |
| 15801 | TTIKACRLKT GCEYQFRIAA ENRYGKSTYL NSEPTVAQYP |
| 15841 | FKVPGPPGTP VVTLSSRDSM EVQWNEPISD GGSRVIGYHL |
| 15881 | ERKERNSILW VKLNKTPIPQ TKFKTTGLEE GVEYEFRVSA |
| 15921 | ENIVGIGKPS KVSECYVARD PCDPPGRPEA IIVTRNSVTL |
| 15961 | QWKKPTYDGG SKITGYIVEK KELPEGRWMK ASFTNIIDTH |
| 16001 | FEVTGLVEDH RYEFRVIARN AAGVFSEPSE STGAITARDE |
| 16041 | VDPPRISMDP KYKDTIVVHA GESFKVDADI YGKPIPTIQW |
| 16081 | IKGDQELSNT ARLEIKSTDF ATSLSVKDAV RVDSGNYILK |
| 16121 | AKNVAGERSV TVNVKVLDRP GPPEGPVVIS GVTAEKCTLA |
| 16161 | WKPPLQDGGS DIINYIVERR ETSRLVWTVV DANVQTLSCK |
| 16201 | VTKLLEGNEY TFRIMAVNKY GVGEPLESEP VVAKNPFVVP |
| 16241 | DAPKAPEVTT VTKDSMIVVW ERPASDGGSE ILGYVLEKRD |
| 16281 | KEGIRWTRCH KRLIGELRLR VTGLIENHDY EFRVSAENAA |
| 16321 | GLSEPSPPSA YQKACDPIYK PGPPNNPKVI DITRSSVFLS |
| 16361 | WSKPIYDGGC EIQGYIVEKC DVSVGEWTMC TPPTGINKTN |
| 16401 | IEVEKLLEKH EYNFRICAIN KAGVGEHADV PGPIIVEEKL |
| 16441 | EAPDIDLDLE LRKIINIRAG GSLRLFVPIK GRPTPEVKWG |
| 16481 | KVDGEIRDAA IIDVTSSFTS LVLDNVNRYD SGKYTLTLEN |
| 16521 | SSGTKSAFVT VRVLDTPSPP VNLKVTEITK DSVSITWEPP |
| 16561 | LLDGGSKIKN YIVEKREATR KSYAAVVTNC HKNSWKIDQL |
| 16601 | QEGCSYYFRV TAENEYGIGL PAQTADPIKV AEVPQPPGKI |
| 16641 | TVDDVTRNSV SLSWTKPEHD GGSKIIQYIV EMQAKHSEKW |
| 16681 | SECARVKSLQ AVITNLTQGE EYLFRVVAVN EKGRSDPRSL |
| 16721 | AVPIVAKDLV IEPDVKPAFS SYSVQVGQDL KIEVPISGRP |
| 16761 | KPTITWTKDG LPLKQTTRIN VTDSLDLTTL SIKETHKDDG |
| 16801 | GQYGITVANV VGQKTASIEI VTLDKPDPPK GPVKFDDVSA |
| 16841 | ESITLSWNPP LYTGGCQITN YIVQKRDTTT TVWDVVSATV |
| 16881 | ARTTLKVTKL KTGTEYQFRI FAENRYGQSF ALESDPIVAQ |
| 16921 | YPYKEPGPPG TPFATAISKD SMVIQWHEPV NNGGSPVIGY |
| 16961 | HLERKERNSI LWTKVNKTII HDTQFKAQNL EEGIEYEFRV |
| 17001 | YAENIVGVGK ASKNSECYVA RDPCDPPGTP EPIMVKRNEI |
| 17041 | TLQWTKPVYD GGSMITGYIV EKRDLPDGRW MKASFTNVIE |
| 17081 | TQFTVSGLTE DQRYEFRVIA KNAAGAISKP SDSTGPITAK |
| 17121 | DEVELPRISM DPKFRDTIVV NAGETFRLEA DVHGKPLPTI |
| 17161 | EWLRGDKEIE ESARCEIKNT DFKALLIVKD AIRIDGGQYI |
| 17201 | LRASNVAGSK SFPVNVKVLD RPGPPEGPVQ VTGVTSEKCS |
| 17241 | LTWSPPLQDG GSDISHYVVE KRETSRLAWT VVASEVVTNS |
| 17281 | LKVTKLLEGN EYVFRIMAVN KYGVGEPLES APVLMKNPFV |
| 17321 | LPGPPKSLEV TNIAKDSMTV CWNRPDSDGG SEIIGYIVEK |
| 17361 | RDRSGIRWIK CNKRRITDLR LRVTGLTEDH EYEFRVSAEN |
| 17401 | AAGVGEPSPA TVYYKACDPV FKPGPPTNAH IVDTTKNSIT |
| 17441 | LAWGKPIYDG GSEILGYVVE ICKADEEEWQ IVTPQTGLRV |
| 17481 | TRFEISKLTE HQEYKIRVCA LNKVGLGEAT SVPGTVKPED |
| 17521 | KLEAPELDLD SELRKGIVVR AGGSARIHIP FKGRPTPEIT |
| 17561 | WSREEGEFTD KVQIEKGVNY TQLSIDNCDR NDAGKYILKL |
| 17601 | ENSSGSKSAF VTVKVLDTPG PPQNLAVKEV RKDSAFLVWE |
| 17641 | PPIIDGGAKV KNYVIDKRES TRKAYANVSS KCSKTSFKVE |
| 17681 | NLTEGAIYYF RVMAENEFGV GVPVETVDAV KAAEPPSPPG |
| 17721 | KVTLTDVSQT SASLMWEKPE HDGGSRVLGY VVEMQPKGTE |
| 17761 | KWSIVAESKV CNAVVTGLSS GQEYQFRVKA YNEKGKSDPR |
| 17801 | VLGVPVIAKD LTIQPSLKLP FNTYSIQAGE DLKIEIPVIG |
| 17841 | RPRPNISWVK DGEPLKQTTR VNVEETATST VLHIKEGNKD |
| 17881 | DFGKYTVTAT NSAGTATENL SVIVLEKPGP PVGPVREDEV |
| 17921 | SADFVVISWE PPAYTGGCQI SNYIVEKRDT TTTTWHMVSA |
| 17961 | TVARTTIKIT KLKTGTEYQF RIFAENRYGK SAPLDSKAVI |
| 18001 | VQYPFKEPGP PGTPFVTSIS KDQMLVQWHE PVNDGGTKII |
| 18041 | GYHLEQKEKN SILWVKLNKT PIQDTKFKTT GLDEGLEYEF |
| 18081 | KVSAENIVGI GKPSKVSECF VARDPCDPPG RPEAIVITRN |
| 18121 | NVTLKWKKPA YDGGSKITGY IVEKKDLPDG RWMKASFTNV |
| 18161 | LETEFTVSGL VEDQRYEFRV IARNAAGNES EPSDSSGAIT |
| 18201 | ARDEIDAPNA SLDPKYKDVI VVHAGETFVL EADIRGKPIP |
| 18241 | DVVWSKDGKE LEETAARMEI KSTIQKTTLV VKDCIRTDGG |
| 18281 | QYILKLSNVG GTKSIPITVK VLDRPGPPEG PLKVTGVTAE |
| 18321 | KCYLAWNPPL QDGGANISHY IIEKRETSRL SWTQVSTEVQ |
| 18361 | ALNYKVTKLL PGNEYIFRVM AVNKYGIGEP LESGPVTACN |
| 18401 | PYKPPGPPST PEVSAITKDS MVVTWARPVD DGGTEIEGYI |
| 18441 | LEKRDKEGVR WTKCNKKTLT DLRLRVTGLT EGHSYEFRVA |
| 18481 | AENAAGVGEP SEPSVFYRAC DALYPPGPPS NPKVTDTSRS |
| 18521 | SVSLAWSKPI YDGGAPVKGY VVEVKEAAAD EWTTCTPPTG |
| 18561 | LQGKQFTVTK LKENTEYNFR ICAINSEGVG EPATLPGSVV |
| 18601 | AQERIEPPEI ELDADLRKVV VLRASATLRL FVTIKGRPEP |
| 18641 | EVKWEKAEGI LTDRAQIEVT SSFTMLVIDN VTRFDSGRYN |
| 18681 | LTLENNSGSK TAFVNVRVLD SPSAPVNLTI REVKKDSVTL |
| 18721 | SWEPPLIDGG AKITNYIVEK RETTRKAYAT ITNNCTKTTF |
| 18761 | RIENLQEGCS YYFRVLASNE YGIGLPAETT EPVKVSEPPL |
| 18801 | PPGRVTLVDV TRNTATIKWE KPESDGGSKI TGYVVEMQTK |
| 18841 | GSEKWSTCTQ VKTLEATISG LTAGEEYVFR VAAVNEKGRS |
| 18881 | DPRQLGVPVI ARDIEIKPSV ELPFHTFNVK AREQLKIDVP |
| 18921 | FKGRPQATVN WRKDGQTLKE TTRVNVSSSK TVTSLSIKEA |
| 18961 | SKEDVGTYEL CVSNSAGSIT VPITIIVLDR PGPPGPIRID |
| 19001 | EVSCDSITIS WNPPEYDGGC QISNYIVEKK ETTSTTWHIV |
| 19041 | SQAVARTSIK IVRLTTGSEY QFRVCAENRY GKSSYSESSA |
| 19081 | VVAEYPFSPP GPPGTPKVVH ATKSTMLVTW QVPVNDGGSR |
| 19121 | VIGYHLEYKE RSSILWSKAN KILIADTQMK VSGLDEGLMY |
| 19161 | EYRVYAENIA GIGKCSKSCE PVPARDPCDP PGQPEVTNIT |
| 19201 | RKSVSLKWSK PHYDGGAKIT GYIVERRELP DGRWLKCNYT |
| 19241 | NIQETYFEVT ELTEDQRYEF RVFARNAADS VSEPSESTGP |
| 19281 | IIVKDDVEPP RVMMDVKFRD VIVVKAGEVL KINADIAGRP |
| 19321 | LPVISWAKDG IEIEERARTE IISTDNHTLL TVKDCIRRDT |
| 19361 | GQYVLTLKNV AGTRSVAVNC KVLDKPGPPA GPLEINGLTA |
| 19401 | EKCSLSWGRP QEDGGADIDY YIVEKRETSH LAWTICEGEL |
| 19441 | QMTSCKVTKL LKGNEYIFRV TGVNKYGVGE PLESVAIKAL |
| 19481 | DPFTVPSPPT SLEITSVTKE SMTLCWSRPE SDGGSEISGY |
| 19521 | IIERREKNSL RWVRVNKKPV YDLRVKSTGL REGCEYEYRV |
| 19561 | YAENAAGLSL PSETSPLIRA EDPVFLPSPP SKPKIVDSGK |
| 19601 | TTITIAWVKP LFDGGAPITG YTVEYKKSDD TDWKTSIQSL |
| 19641 | RGTEYTISGL TTGAEYVFRV KSVNKVGASD PSDSSDPQIA |
| 19681 | KEREEEPLFD IDSEMRKTLI VKAGASFTMT VPFRGRPVPN |
| 19721 | VLWSKPDTDL RTRAYVDTTD SRTSLTIENA NRNDSGKYTL |
| 19761 | TIQNVLSAAS LTLVVKVLDT PGPPTNITVQ DVTKESAVLS |
| 19801 | WDVPENDGGA PVKNYHIEKR EASKKAWVSV TNNCNRLSYK |
| 19841 | VTNLQEGAIY YFRVSGENEF GVGIPAETKE GVKITEKPSP |
| 19881 | PEKLGVTSIS KDSVSLTWLK PEHDGGSRIV HYVVEALEKG |
| 19921 | QKNWVKCAVA KSTHHVVSGL RENSEYFFRV FAENQAGLSD |
| 19961 | PRELLLPVLI KEQLEPPEID MKNFPSHTVY VRAGSNLKVD |
| 20001 | IPISGKPLPK VTLSRDGVPL KATMRENTEI TAENLTINLK |
| 20041 | ESVTADAGRY EITAANSSGT TKAFINIVVL DRPGPPTGPV |
| 20081 | VISDITEESV TLKWEPPKYD GGSQVTNYIL LKRETSTAVW |
| 20121 | TEVSATVART MMKVMKLTTG EEYQFRIKAE NRFGISDHID |
| 20161 | SACVTVKLPY TTPGPPSTPW VTNVTRESIT VGWHEPVSNG |
| 20201 | GSAVVGYHLE MKDRNSILWQ KANKLVIRTT HFKVTTISAG |
| 20241 | LIYEFRVYAE NAAGVGKPSH PSEPVLAIDA CEPPRNVRIT |
| 20281 | DISKNSVSLS WQQPAFDGGS KITGYIVERR DLPDGRWTKA |
| 20321 | SFTNVTETQF IISGLTQNSQ YEFRVFARNA VGSISNPSEV |
| 20361 | VGPITCIDSY GGPVIDLPLE YTEVVKYRAG TSVKLRAGIS |
| 20401 | GKPAPTIEWY KDDKELQTNA LVCVENTTDL ASILIKDADR |
| 20441 | LNSGCYELKL RNAMGSASAT IRVQILDKPG PPGGPIEFKT |
| 20481 | VTAEKITLLW RPPADDGGAK ITHYIVEKRE TSRVVWSMVS |
| 20521 | EHLEECIITT TKIIKGNEYI FRVRAVNKYG IGEPLESDSV |
| 20561 | VAKNAFVTPG PPGIPEVTKI TKNSMTVVWS RPIADGGSDI |
| 20601 | SGYFLEKRDK KSLGWFKVLK ETIRDTRQKV TGLTENSDYQ |
| 20641 | YRVCAVNAAG QGPFSEPSEF YKAADPIDPP GPPAKIRIAD |
| 20681 | STKSSITLGW SKPVYDGGSA VTGYVVEIRQ GEEEEWTTVS |
| 20721 | TKGEVRTTEY VVSNLKPGVN YYFRVSAVNC AGQGEPIEMN |
| 20761 | EPVQAKDILE APEIDLDVAL RTSVIAKAGE DVQVLIPFKG |
| 20801 | RPPPTVTWRK DEKNLGSDAR YSIENTDSSS LLTIPQVTRN |
| 20841 | DTGKYILTIE NGVGEPKSST VSVKVLDTPA ACQKLQVKHV |
| 20881 | SRGTVTLLWD PPLIDGGSPI INYVIEKRDA TKRTWSVVSH |
| 20921 | KCSSTSFKLI DLSEKTPFFF RVLAENEIGI GEPCETTEPV |
| 20961 | KAAEVPAPIR DLSMKDSTKT SVILSWTKPD FDGGSVITEY |
| 21001 | VVERKGKGEQ TWSHAGISKT CEIEVSQLKE QSVLEFRVFA |
| 21041 | KNEKGLSDPV TIGPITVKEL IITPEVDLSD IPGAQVTVRI |
| 21081 | GHNVHLELPY KGKPKPSISW LKDGLPLKES EFVRFSKTEN |
| 21121 | KITLSIKNAK KEHGGKYTVI LDNAVCRIAV PITVITLGPP |
| 21161 | SKPKGPIRFD EIKADSVILS WDVPEDNGGG EITCYSIEKR |
| 21201 | ETSQTNWKMV CSSVARTTFK VPNLVKDAEY QFRVRAENRY |
| 21241 | GVSQPLVSSI IVAKHQFRIP GPPGKPVIYN VTSDGMSLTW |
| 21281 | DAPVYDGGSE VTGFHVEKKE RNSILWQKVN TSPISGREYR |
| 21321 | ATGLVEGLDY QFRVYAENSA GLSSPSDPSK FTLAVSPVDP |
| 21361 | PGTPDYIDVT RETITLKWNP PLRDGGSKIV GYSIEKRQGN |
| 21401 | ERWVRCNFTD VSECQYTVTG LSPGDRYEFR IIARNAVGTI |
| 21441 | SPPSQSSGII MTRDENVPPI VEFGPEYFDG LIIKSGESLR |
| 21481 | IKALVQGRPV PRVTWFKDGV EIEKRMNMEI TDVLGSTSLF |
| 21521 | VRDATRDHRG VYTVEAKNAS GSAKAEIKVK VQDTPGKVVG |
| 21561 | PIRFTNITGE KMTLWWDAPL NDGCAPITHY IIEKRETSRL |
| 21601 | AWALIEDKCE AQSYTAIKLI NGNEYQFRVS AVNKFGVGRP |
| 21641 | LDSDPVVAQI QYTVPDAPGI PEPSNITGNS ITLTWARPES |
| 21681 | DGGSEIQQYI LERREKKSTR WVKVISKRPI SETRFKVTGL |
| 21721 | TEGNEYEFHV MAENAAGVGP ASGISRLIKC REPVNPPGPP |
| 21761 | TVVKVTDTSK TTVSLEWSKP VEDGGMEIIG YIIEMCKADL |
| 21801 | GDWHKVNAEA CVKTRYTVTD LQAGEEYKFR VSAINGAGKG |
| 21841 | DSCEVTGTIK AVDRLTAPEL DIDANFKQTH VVRAGASIRL |
| 21881 | FIAYQGRPTP TAVWSKPDSN LSLRADIHTT DSFSTLTVEN |
| 21921 | CNRNDAGKYT LTVENNSGSK SITFTVKVLD TPGPPGPITF |
| 21961 | KDVTRGSATL MWDAPLLDGG ARIHHYVVEK REASRRSWQV |
| 22001 | ISEKCTRQIF KVNDLAEGVP YYFRVSAVNE YGVGEPYEMP |
| 22041 | EPIVATEQPA PPRRLDVVDT SKSSAVLAWL KPDHDGGSRI |
| 22081 | TGYLLEMRQK GSDFWVEAGH TKQLTFTVER LVEKTEYEFR |
| 22121 | VKAKNDAGYS EPREAFSSVI IKEPQIEPTA DLTGITNQLI |
| 22161 | TCKAGSPFTI DVPISGRPAP KVTWKLEEMR LKETDRVSIT |
| 22201 | TTKDRTTLTV KDSMRGDSGR YFLTLENTAG VKTFSVTVVV |
| 22241 | IGRPGPVTGP IEVSSVSAES CVLSWGEPKD GGGTEITNYI |
| 22281 | VEKRESGTTA WQLVNSSVKR TQIKVTHLTK YMEYSFRVSS |
| 22321 | ENRFGVSKPL ESAPIIAEHP FVPPSAPTRP EVYHVSANAM |
| 22361 | SIRWEEPYHD GGSKIIGYWV EKKERNTILW VKENKVPCLE |
| 22401 | CNYKVTGLVE GLEYQFRTYA LNAAGVSKAS EASRPIMAQN |
| 22441 | PVDAPGRPEV TDVTRSTVSL IWSAPAYDGG SKVVGYIIER |
| 22481 | KPVSEVGDGR WLKCNYTIVS DNFFTVTALS EGDTYEFRVL |
| 22521 | AKNAAGVISK GSESTGPVTC RDEYAPPKAE LDARLHGDLV |
| 22561 | TIRAGSDLVL DAAVGGKPEP KIIWTKGDKE LDLCEKVSLQ |
| 22601 | YTGKRATAVI KFCDRSDSGK YTLTVKNASG TKAVSVMVKV |
| 22641 | LDSPGPCGKL TVSRVTQEKC TLAWSLPQED GGAEITHYIV |
| 22681 | ERRETSRLNW VIVEGECPTL SYVVTRLIKN NEYIFRVRAV |
| 22721 | NKYGPGVPVE SEPIVARNSF TIPSPPGIPE EVGTGKEHII |
| 22761 | IQWTKPESDG GNEISNYLVD KREKKSLRWT RVNKDYVVYD |
| 22801 | TRLKVTSLME GCDYQFRVTA VNAAGNSEPS EASNFISCRE |
| 22841 | PSYTPGPPSA PRVVDTTKHS ISLAWTKPMY DGGTDIVGYV |
| 22881 | LEMQEKDTDQ WYRVHTNATI RNTEFTVPDL KMGQKYSFRV |
| 22921 | AAVNVKGMSE YSESIAEIEP VERIEIPDLE LADDLKKTVT |
| 22961 | IRAGASLRLM VSVSGRPPPV ITWSKQGIDL ASRAIIDTTE |
| 23001 | SYSLLIVDKV NRYDAGKYTI EAENQSGKKS ATVLVKVYDT |
| 23041 | PGPCPSVKVK EVSRDSVTIT WEIPTIDGGA PVNNYIVEKR |
| 23081 | EAAMRAFKTV TTKCSKTLYR ISGLVEGTMY YFRVLPENIY |
| 23121 | GIGEPCETSD AVLVSEVPLV PAKLEVVDVT KSTVTLAWEK |
| 23161 | PLYDGGSRLT GYVLEACKAG TERWMKVVTL KPTVLEHTVT |
| 23201 | SLNEGEQYLF RIRAQNEKGV SEPRETVTAV TVQDLRVLPT |
| 23241 | IDLSTMPQKT IHVPAGRPVE LVIPIAGRPP PAASWFFAGS |
| 23281 | KLRESERVTV ETHTKVAKLT IRETTIRDTG EYTLELKNVT |
| 23321 | GTTSETIKVI ILDKPGPPTG PIKIDEIDAT SITISWEPPE |
| 23361 | LDGGAPLSGY VVEQRDAHRP GWLPVSESVT RSTFKFTRLT |
| 23401 | EGNEYVFRVA ATNRFGIGSY LQSEVIECRS SIRIPGPPET |
| 23441 | LQIFDVSRDG MTLTWYPPED DGGSQVTGYI VERKEVRADR |
| 23481 | WVRVNKVPVT MTRYRSTGLT EGLEYEHRVT AINARGSGKP |
| 23521 | SRPSKPIVAM DPIAPPGKPQ NPRVTDTTRT SVSLAWSVPE |
| 23561 | DEGGSKVTGY LIEMQKVDQH EWTKCNTTPT KIREYTLTHL |
| 23601 | PQGAEYRFRV LACNAGGPGE PAEVPGTVKV TEMLEYPDYE |
| 23641 | LDERYQEGIF VRQGGVIRLT IPIKGKPFPI CKWTKEGQDI |
| 23681 | SKRAMIATSE THTELVIKEA DRGDSGTYDL VLENKCGKKA |
| 23721 | VYIKVRVIGS PNSPEGPLEY DDIQVRSVRV SWRPPADDGG |
| 23761 | ADILGYILER REVPKAAWYT IDSRVRGTSL VVKGLKENVE |
| 23801 | YHFRVSAENQ FGISKPLKSE EPVTPKTPLN PPEPPSNPPE |
| 23841 | VLDVTKSSVS LSWSRPKDDG GSRVTGYYIE RKETSTDKWV |
| 23881 | RHNKTQITTT MYTVTGLVPD AEYQFRIIAQ NDVGLSETSP |
| 23921 | ASEPVVCKDP FDKPSQPGEL EILSISKDSV TLQWEKPECD |
| 23961 | GGKEILGYWV EYRQSGDSAW KKSNKERIKD KQFTIGGLLE |
| 24001 | ATEYEFRVFA ENETGLSRPR RTAMSIKTKL TSGEAPGIRK |
| 24041 | EMKDVTTKLG EAAQLSCQIV GRPLPDIKWY RFGKELIQSR |
| 24081 | KYKMSSDGRT HTLTVMTEEQ EDEGVYTCIA TNEVGEVETS |
| 24121 | SKLLLQATPQ FHPGYPLKEK YYGAVGSTLR LHVMYIGRPV |
| 24161 | PAMTWFHGQK LLQNSENITI ENTEHYTHLV MKNVQRKTHA |
| 24201 | GKYKVQLSNV FGTVDAILDV EIQDKPDKPT GPIVIEALLK |
| 24241 | NSAVISWKPP ADDGGSWITN YVVEKCEAKE GAEWQLVSSA |
| 24281 | ISVTTCRIVN LTENAGYYFR VSAQNTFGIS DPLEVSSVVI |
| 24321 | IKSPFEKPGA PGKPTITAVT KDSCVVAWKP PASDGGAKIR |
| 24361 | NYYLEKREKK QNKWISVTTE EIRETVFSVK NLIEGLEYEF |
| 24401 | RVKCENLGGE SEWSEISEPI TPKSDVPIQA PHFKEELRNL |
| 24441 | NVRYQSNATL VCKVTGHPKP IVKWYRQGKE IIADGLKYRI |
| 24481 | QEFKGGYHQL IIASVTDDDA TVYQVRATNQ GGSVSGTASL |
| 24521 | EVEVPAKIHL PKTLEGMGAV HALRGEVVSI KIPFSGKPDP |
| 24561 | VITWQKGQDL IDNNGHYQVI VTRSFTSLVF PNGVERKDAG |
| 24601 | FYVVCAKNRF GIDQKTVELD VADVPDPPRG VKVSDVSRDS |
| 24641 | VNLTWTEPAS DGGSKITNYI VEKCATTAER WLRVGQARET |
| 24681 | RYTVINLFGK TSYQFRVIAE NKFGLSKPSE PSEPTITKED |
| 24721 | KTRAMNYDEE VDETREVSMT KASHSSTKEL YEKYMIAEDL |
| 24761 | GRGEFGIVHR CVETSSKKTY MAKFVKVKGT DQVLVKKEIS |
| 24801 | ILNIARHRNI LHLHESFESM EELVMIFEFI SGLDIFERIN |
| 24841 | TSAFELNERE IVSYVHQVCE ALQFLHSHNI GHFDIRPENI |
| 24881 | IYQTRRSSTI KIIEFGQARQ LKPGDNFRLL FTAPEYYAPE |
| 24921 | VHQHDVVSTA TDMWSLGTLV YVLLSGINPF LAETNQQIIE |
| 24961 | NIMNAEYTFD EEAFKEISIE AMDFVDRLLV KERKSRMTAS |
| 25001 | EALQHPWLKQ KIERVSTKVI RTLKHRRYYH TLIKKDLNMV |
| 25041 | VSAARISCGG AIRSQKGVSV AKVKVASIEI GPVSGQIMHA |
| 25081 | VGEEGGHVKY VCKIENYDQS TQVTWYFGVR QLENSEKYEI |
| 25121 | TYEDGVAILY VKDITKLDDG TYRCKVVNDY GEDSSYAELF |
| 25161 | VKGVREVYDY YCRRTMKKIK RRTDTMRLLE RPPEFTLPLY |
| 25201 | NKTAYVGENV RFGVTITVHP EPHVTWYKSG QKIKPGDNDK |
| 25241 | KYTFESDKGL YQLTINSVTT DDDAEYTVVA RNKYGEDSCK |
| 25281 | AKLTVTLHPP PTDSTLRPMF KRLLANAECQ EGQSVCFEIR |
| 25321 | VSGIPPPTLK WEKDGQPLSL GPNIEIIHEG LDYYALHIRD |
| 25361 | TLPEDTGYYR VTATNTAGST SCQAHLQVER LRYKKQEFKS |
| 25401 | KEEHERHVQK QIDKTLRMAE ILSGTESVPL TQVAKEALRE |
| 25441 | AAVLYKPAVS TKTVKGEFRL EIEEKKEERK LRMPYDVPEP |
| 25481 | RKYKQTTIEE DQRIKQFVPM SDMKWYKKIR DQYEMPGKLD |
| 25521 | RVVQKRPKRI RLSRWEQFYV MPLPRITDQY RPKWRIPKLS |
| 25561 | QDDLEIVRPA RRRTPSPDYD FYYRPRRRSL GDISDEELLL |
| 25601 | PIDDYLAMKR TEEERLRLEE ELELGFSASP PSRSPPHFEL |
| 25641 | SSLRYSSPQA HVKVEETRKD FRYSTYHIPT KAEASTSYAE |
| 25681 | LRERHAQAAY RQPKQRQRIM AEREDEELLR PVTTTQHLSE |
| 25721 | YKSELDFMSK EEKSRKKSRR QREVTEITEI EEEYEISKHA |
| 25761 | QRESSSSASR LLRRRRSLSP TYIELMRPVS ELIRSRPQPA |
| 25801 | EEYEDDTERR SPTPERTRPR SPSPVSSERS LSRFERSARF |
| 25841 | DIFSRYESMK AALKTQKTSE RKYEVLSQQP FTLDHAPRIT |
| 25881 | LRMRSHRVPC GQNTRFILNV QSKPTAEVKW YHNGVELQES |
| 25921 | SKIHYTNTSG VLTLEILDCH TDDSGTYRAV CTNYKGEASD |
| 25961 | YATLDVTGGD YTTYASQRRD EEVPRSVFPE LTRTEAYAVS |
| 26001 | SFKKTSEMEA SSSVREVKSQ MTETRESLSS YEHSASAEMK |
| 26041 | SAALEEKSLE EKSTTRKIKT TLAARILTKP RSMTVYEGES |
| 26081 | ARFSCDTDGE PVPTVTWLRK GQVLSTSARH QVTTTKYKST |
| 26121 | FEISSVQASD EGNYSVVVEN SEGKQEAEFT LTIQKARVTE |
| 26161 | KAVTSPPRVK SPEPRVKSPE AVKSPKRVKS PEPSHPKAVS |
| 26201 | PTETKPTPTE KVQHLPVSAP PKITQFLKAE ASKEIAKLTC |
| 26241 | VVESSVLRAK EVTWYKDGKK LKENGHFQFH YSADGTYELK |
| 26281 | INNLTESDQG EYVCEISGEG GTSKTNLQFM GQAFKSIHEK |
| 26321 | VSKISETKKS DQKTTESTVT RKTEPKAPEP ISSKPVIVTG |
| 26361 | LQDTTVSSDS VAKFAVKATG EPRPTAIWTK DGKAITQGGK |
| 26401 | YKLSEDKGGF FLEIHKTDTS DSGLYTCTVK NSAGSVSSSC |
| 26441 | KLTIKAIKDT EAQKVSTQKT SEITPQKKAV VQEEISQKAL |
| 26481 | RSEEIKMSEA KSQEKLALKE EASKVLISEE VKKSAATSLE |
| 26521 | KSIVHEEITK TSQASEEVRT HAEIKAFSTQ MSINEGQRLV |
| 26561 | LKANIAGATD VKWVLNGVEL TNSEEYRYGV SGSDQTLTIK |
| 26601 | QASHRDEGIL TCISKTKEGI VKCQYDLTLS KELSDAPAFI |
| 26641 | SQPRSQNINE GQNVLFTCEI SGEPSPEIEW FKNNLPISIS |
| 26681 | SNVSISRSRN VYSLEIRNAS VSDSGKYTIK AKNFRGQCSA |
| 26721 | TASLMVLPLV EEPSREVVLR TSGDTSLQGS FSSQSVQMSA |
| 26761 | SKQEASFSSF SSSSASSMTE MKFASMSAQS MSSMQESFVE |
| 26801 | MSSSSFMGIS NMTQLESSTS KMLKAGIRGI PPKIEALPSD |
| 26841 | ISIDEGKVLT VACAFTGEPT PEVTWSCGGR KIHSQEQGRF |
| 26881 | HIENTDDLTT LIIMDVQKQD GGLYTLSLGN EFGSDSATVN |
| 26921 | IHIRSI |

In some cases, SARS-CoV-2 infection can be monitored by observing cleavage of titin in the C-terminal region. For example, such cleavage can occur in the M-band (also called the M-line) region of titin. The M band is at the C-terminal end 35 of the titin protein and in the center of the A band, which is in the center of the sarcomere. The approximate 250 kilodalton M band is an attachment site for the thick filaments, and it is encoded by six exons, exons 359 to 364, which are also termed M-band exons 1 to 6 (Mex1 to Mex6). The M band region interacts with several sarcomeric proteins including myosin-binding protein C, calmodulin 1, CAPN3, obscurin, and MURF1.

Cleavage of titin can be observed within the C-terminal 2000-4000 amino acids, or the 2000-3000 amino acids of the titin protein. Such cleavage is observed when SARS-CoV-2 infection occurs. A test agent that causes a reduction in titin cleavage (e.g., compared to a control) can be useful for treating and/or preventing SARS-CoV-2 infection.

### COVID-19

Initial descriptions of COVID-19, the pandemic disease caused by the severe acute respiratory syndrome coronavirus 2 (SARS-CoV-2), characterized it as a primarily respiratory syndrome (see website at pubmed.ncbi.nlm.nih.gov/32031570/). However, increasing clinical evidence now implicates multiple organ systems in COVID-19 infection, including the heart, gastrointestinal tract, and kidneys (Wang, see websites at sciencedirect.com/science/article/pii/S0140673620302117; ahajournals.org/doi/10.1161/CIRCULATIONAHA.120.047164; jamanetwork.com/journals/jama/fullarticle/2763485; jamanetwork.com/joumals/jama/fullarticle/2765184).

As illustrated herein, multiple COVID-19 patients frequently present with significant myocardial damage (see also websites at jamanetwork.com/journals/jamacardiology/fullarticle/2763845; academic.oup.com/cardiovascres/article/116/10/1666/5826160; nature.com/articles/s41569-020-0413-9), even when they exhibited no prior cardiovascular disease (CVD) (jamanetwork.com/journals/jamacardiology/fullarticle/2763524), indicating that viral infection may be directly responsible for the cardiac damage. Meta-analyses identify elevated high-sensitivity troponin-I or natriuretic peptides as the strongest predictor of mortality in hospitalized patients, eclipsing both cardiovascular disease and congestive obstructive pulmonary disease (see websites at thelancet.com/journals/lancet/article/PIIS0140-6736(20)30566-3/fulltext; pubmed.ncbi.nlm.nih.gov/32362922/; pubmed.ncbi.nlm.nih.gov/32125452/; jamanetwork.com/joumals/jamacardiology/fullarticle/2763524). Alarmingly, evidence of elevated troponin can be found even in mild cases of COVID-19, and a recent study observed that the majority of recovered patients in the studied cohort presented with impaired cardiac function, indicating that long-term heart sequelae from COVID-19 may not be limited to intensive care unit cases (see website atjamanetwork.com/joumals/jamacardiology/fullarticle/2768916).

Identifying therapeutic strategies to prevent or manage myocardial injury in COVID-19 patients is hindered by limited understanding of the mechanisms by which SARS-CoV-2 induces cardiac damage. Besides direct myocardial infection, cardiac damage may be caused by other systemic impacts of SARS-CoV-2, such as hypoxic stress due to pulmonary damage, microvascular thrombosis, and/or the systemic immune response to viral infection (see website at ncbi.nlm.nih.gov/pmc/articles/PMC7270045/). Recent histological results from deceased COVID-19 patients detect viral RNA in the myocardium without inflammatory cell infiltrates (see website at jamanetwork.com/joumals/jamacardiology/fullarticle/2768914), but whether these transcripts arise from infected myocytes, cardiac stroma, or blood vessels was previously unknown (see website at onlinelibrary.wiley.com/doi/abs/10.1002/ejhf.1828). Cardiomyocytes are known to express the primary receptor for viral entry, ACE2 (see website at sciencedirect.com/science/article/pii/S0092867420302294) and may be infectable by SARS-CoV-2 (see website at ahajournals.org/doi/full/10.1161/CIRCULATIONAHA.120.047549). Developing effective interventions for cardiac injury in COVID-19 requires identification of the key molecules and cell types involved in mediating viral infection and cellular anomalies.

As described herein, ex vivo studies employed using human cell-based models of the heart were used to afford the most direct route for the prospective and clinically relevant study of the effects of cardiac viral infection. Human induced pluripotent stem cells (iPSCs) can be used as described herein to obtain functional cardiac tissue models for disease modeling and discovery, overcoming the infeasibility of using primary human hearts. Stem-cell derived models have already demonstrated the susceptibility of hepatocytes (see website at sciencedirect.com/science/article/pii/S1934590920302824), intestinal epithelium (see website at nature.com/articles/s41591-020-0912-6; see website at ncbi.nlm.nih.gov/pmc/articles/PMC7199907/), and lung organoids (see website at biorxiv.org/content/10.1101/2020.05.05.079095v1. abstract) to SARS-CoV-2 infection.

While two recent reports indicated that human iPSC-cardiomyocytes are susceptible to SARS-CoV-2 infection (see websites at cell.com/cell-reports-medicine/fulltext/S2666-3791(20)30068-9, biorxiv.org/content/10.1101/2020.06.01.127605v1), clear indications of specific cardiac cytopathic features have not been identified. In addition, the relative viral tropism for other cardiac cell types that may be involved in microthrombosis or weakening of the ventricular wall has previously not been explored, nor has there been direct correlation of in vitro results to clinical pathology specimens.

Identifying phenotypic biomarkers of SARS-CoV-2 infection and cardiac cytopathy that recapitulate features of patient tissue is critical for rapidly developing novel cardioprotective therapies efficacious against COVID-19. As described herein, the inventors have examined the relative susceptibility of three iPS-derived cardiac cell types: cardiomyocytes (CMs), cardiac fibroblasts (CFs), and endothelial cells (ECs), to SARS-CoV-2 infection, and identify clear hallmarks of infection and cardiac cytopathy that predict pathologic features found in human COVID-19 tissue specimens.

### Definitions

The term “about” as used herein when referring to a measurable value such as an amount, a length, and the like, is meant to encompass variations of 20% or +10%, more preferably 5%, even more preferably 1%, and still more preferably 0.1% from the specified value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

“Recombinant” as used herein to describe a nucleic acid molecule means a polynucleotide of genomic, cDNA, bacterial, mammalian, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the gene of interest is cloned and then expressed in transformed organisms. The host organism expresses the foreign gene to produce the protein under expression conditions.

As used herein, a “cell” refers to any type of cell. The cell can be in an organism or it can be maintained outside of an organism. The cell can be within a living organism and be in its normal (native) state. The term “cell” includes an individual cell or a group or population of cells. The cell(s) can be a prokaryotic, eukaryotic, or archaeon cell(s), such as a bacterial, archaeal, fungal, protist, plant, or animal cell(s). The cell(s) can be from or be within tissues, organs, and biopsies. The cell(s) can be a recombinant cell(s), a cell(s) from a cell line cultured in vitro. The cell(s) can include cellular fragments, cell components, or organelles comprising nucleic acids. In some cases, the cell(s) are human cells. The term cell(s) also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide into a host cell, irrespective of the method used for the insertion. For example, direct uptake, transfection, or transduction are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected RNA or a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

The term “transduction” refers to the introduction of foreign nucleic acid to a cell through a replication-incompetent viral vector.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Mammalian cell” refers to any cell derived from a mammalian subject suitable for transfection with an engineered vector system comprising an expression system described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The following example illustrate some of the experiments used in the development of the invention and some features of the invention.

### Example 1: Materials and Methods

This Example describes some of the materials and methods used in developing and practicing the invention.

hiPSC Maintenance; iPS-Cardiomyocyte Differentiation and Purification

Human iPS cells (WTC11 line; see website at ncbi.nlm.nih.gov/pmc/articles/PMC4063274/) were maintained in mTESR or mTESR+(STEMCELL Technologies) on Matrigel (8 μg/ml, BD Biosciences)-coated cell culture plates at 37° C., 5% CO2. Cells were passaged every 3 days using Relesr (STEMCELL Technologies) and supplemented with Rock Inhibitor Y-27632 (SelleckChem) for 24 hours after each passaging. hiPSCs were differentiated into cardiomyocytes following a modified Wnt pathway modulation-based GiWi protocol (see website at ncbi.nlm.nih.gov/pmc/articles/PMC3612968/). Briefly, hiPSCs cultures were harvested using Accutase (STEMCELL Technologies) and seeded onto Matrigel-coated 12-well plates. Three days later, cells were exposed to 12 uM CHIR99021 (Tocris) in RPMI1640 (Gibco, 11875093) supplemented with B27 without insulin (Gibco, A1895601) (R/B media) for 24 hours. After an additional 48 hours, media was changed to R/B media supplemented with 5 uM IWP2 (Tocris) for 48 hours. On day 7, media was changed to RPMI1640 medium supplemented with B27 with insulin (Gibco, 17504044) (R/B*) and refreshed every 3 days thereafter. Beating was generally observed around day 8-11. At day 15, cells were cryopreserved using CryoStor CS10 (STEMCELL Technologies). After thawing, cell cultures were enriched for iPS-cardiomyocytes following metabolic switch purification (see website at pubmed.ncbi.nlm.nih.gov/23168164/). Briefly, cells were washed once with saline buffer and incubated in DMEM (without glucose, without sodium pyruvate; Gibco, 11966025) supplemented with GlutaMax (Gibco, 35050061), MEM Non-Essential Amino Acids (Gibco, 11140050) and sodium L-lactate (4 mM, Sigma-Aldrich). Lactate media was refreshed every other day for a total of 6 days. Four to six days later (day 28-30), iPS-CMs were replated into assay plates for infection using 0.25% Trypsin (Gibco, 15050065) at a density of approximately 60,000 cells/cm².

scRNAseq Analysis of SARS-CoV-2 Entry Factors

A historic single cell RNA sequencing data set consisting of iPSC-derived cardiomyocytes, primary fetal cardiac fibroblasts, and iPSC-derived endothelial cells was re-analyzed to compare relative expression levels of SARS-CoV-2 relevant receptors and proteases (GSE155226) (see web at biorxiv.org/content/10.1101/2020.07.06.190504v1). Briefly, day 30 lactate purified cardiomyocytes were force aggregated either alone or with a single supporting cell type. The cardiomyocytes were then cultured in suspension culture. Aggregates were dissociated and libraries prepared using the Chromium 3′ v2 library preparation platform (10× Genomics). Libraries were sequenced on a NextSeq 550 sequencer (Illumina) to a depth of at least 30 million reads per sample. Samples were demultiplexed and aligned to GRCh38 with CellRanger v3.0.2. Samples were normalized and clustered with Seurat v3.2.0, yielding four primary clusters corresponding to each cell type, which were used to profile cell-type specific expression of SARS-CoV-2 relevant factors.

### Cardiac Fibroblast Differentiation

Second heart field-derived cardiac fibroblasts (SHF-CFs) were differentiated following the GiFGF protocol, as described by (website at nature.com/articles/s41467-019-09831-5). Briefly, hiPSCs were seeded at 15,000 cells/cm² in mTeSR1 medium. Once they reached 100% confluency, they were treated with R/B media supplemented with 12 μM CHIR99021 (day 0) and refreshed with R/B media 24 hours later (day 1). From days 2-20, cells were fed every 2 days with cardiac fibroblast basal media (CFBM) (Lonza, CC-3131) supplemented with 75 ng/mL bFGF. On day 20, CFs were singularized with Accutase for 10 minutes and replated at 7,000 cells/cm² onto tissue culture plastic 10 cm dishes in FibroGRO medium (Millipore Sigma, SCMF001). FibroGRO media was changed every two days until the CFs reached approximately 80-90% confluency, at which point they were passaged with Accutase. SHF-CFs were validated to be >80% double-positive for TE-7 and vimentin by flow cytometry.

### Endothelial Cell Differentiation

WTC11 iPSCs were directed towards an endothelial cell (EC) lineage by the addition of E8 media supplemented with BMP4 (5 ng/ml) and Activin A (25 ng/ml) for 48 hours followed by E7BVi media, consisting of E6 medium supplemented with bFGF (50 ng/ml), VEGF-A (50 ng/ml), BMP4 (50 ng/ml) and a TGFβ inhibitor, SB431542, (5 μM) for 72 hours. After 5 days of successive media changes, ECs were split and plated at high density in EGM media (Lonza, CC-3162) on tissue culture flasks coated with fibronectin (1:100, Sigma Aldrich F0895). On day 8, all cells were cryo-preserved and a fraction of ECs were assayed for >95% purity by flow cytometry using antibodies against mature EC markers CD31 and CDH5.

### Mixed Cultures of CMs, CFs, and ECs

Mixed cultures of induced pluripotent stem cell derived cardiomyocytes (iPS-CMs), induced pluripotent stem cell derived endothelial cells (iPS-ECs), and induced pluripotent stem cell derived cardiac fibroblasts (iPS-CFs) were created by combining single cell suspensions of each cell types in a ratio of 60:30:10 CM:EC:CF at a density of 200,000 cells/mL. The mixed suspension was replated onto Matrigel-coated tissue culture plates 48 hours prior to infection at a density of 62,500 cells/cm².

### Reagents

TABLE 1A
Drugs

| Drug | Concentration | | Provider |
|---|---|---|---|
| DMSO | 0.1% (1:1000) | | Fisher Scientific (BP231-100) |
| Interferon α | 2500 | U/mL | Sigma Aldrich (SRP4596-100UG) |
| Interferon β | 2500 | U/mL | Sigma Aldrich (IF014) |
| Interferon γ | 2500 | U/mL | BioRad (PHP050A) |
| Interferon λ | 2500 | U/mL | Cedarlane Laboratories (CLY100- |
| | | | 169-5UG) |
| Ruxolitinib | 500 | nM | Thermo (NC1399519) |
| Doxorubicin | 20 nM and | | Sigma (D1515) |
| | 200 nM | | |
| Bortezomib | 1 uM and 10 uM | | Sigma (CAS 179324-69-7) |
| Dutasteride | 2 | uM | Cayman Chemical (164656-23-9) |
| Albendazole | 2 | uM | Cayman Chemical (54965-21-8) |
| Bafilomycin | 100 | nM | Cayman Chemical (98813-13-9) |
| IL-32 blocking antibody | 50 | ng/mL | R&D Systems (AF3040) |
| IL-8 recombinant | 50 | ng/mL | Sigma (I1645-10UG) |
| IL-32 recombinant | 100 | ng/mL | Thermo Fisher (4690IL025CF) |
| IL-8 blocking antibody | 100 | ng/mL | R&D Systems (MAB208-100) |
| Repaxirin | 1 | uM | MedChem Express (HY-15252) |
| Aprotinin | 50 | uM | Cayman Chemical (9087-70-1) |
| Camostat mesilate | 2 | uM | Cayman Chemical (59721-29-8) |
| CA074 | 30 | uM | Cayman Chemical (134448-10-5) |
| E-64d | 25 | uM | Cayman Chemical (88321-09-9) |
| Z-Phe-Tyr(tBu)- | 30 | uM | Cayman Chemical (114014-15-2) |
| diazomethylketone | | | |
| ACE2 blocking antibody | 20 | ug/ml | R&D Systems (AF933) |
| Cardiac Troponin antibody | 1:200 | | Abcam (ab45932-100 ug) |
| ACTN2 polyclonal antibody | 1:200 | | Life Technologies Corporation |
| | | | (14221-1-AP) |
| Apilimod | 1 | uM | SelleckChem Chemicals (S6414) |

| | | |
|---|---|---|
| Anti-SARS-CoV-2 Spike | 1:200 | Provided by BEI resources (NR-616) |
| protein antibody | | |
| Anti-dsRNA antibody (J2) | 1:200 | Absolute Antibody (Ab01299-2.0) |

### SARS-CoV-2 Infection

The WA-1 strain (BEI resources) of SARS-CoV-2 was used for all experiments. SARS-CoV-2 stocks were passaged in Vero cells (ATCC) and titer was determined via plaque assay on Vero cells as previously described (Honko et al ref). Briefly, virus was diluted 1:10²-1:10⁶ and incubated for 1 hour on Vero cells before an overlay of Avicel and complete DMEM (Sigma Aldrich, SLM-241) was added. After incubation at 37° C. for 72 hours, the overlay was removed and cells were fixed with 10% formalin, stained with crystal violet, and counted for plaque formation. SARS-CoV-2 infections of iPS-derived cardiac cells were done at a multiplicity of infection of 0.006 for 48 hours unless otherwise specified. For heat inactivation, SARS-CoV-2 stocks were incubated at 85° C. for 5 min.

### Immunocytochemistry

Infected and mock-treated cell cultures were washed with Phosphate Buffered Solution (PBS) and fixed in 4% paraformaldehyde (PFA) overnight, followed by blocking and permeabilization with 0.1% Triton-X 100 (T8787, Sigma) and 5% BSA (A4503, Sigma) for one hour at RT. Antibody dilution buffer (Ab buffer) was comprised of PBS supplemented with 0.1% Triton-X 100 and 1% BSA. Samples were incubated with primary antibodies overnight at 4° C. (Table 1), followed by 3 washes with PBS and incubation with fluorescent-conjugated secondary antibodies at 1:250 in Ab buffer for 1 hour at room temperature (Table 1). For immunofluorescence staining, epitopes were retrieved through 35 min incubation at 95° C. in citrate solution (pH 6) or TE buffer (pH 9) and coverslips were mounted onto SuperFrost Slides (FisherBrand, 12-550-15) with ProLong Antifade mounting solution with DAPI (Invitrogen, P36931). Primary antibodies and nuclear stains were used as follows: J2 (Absolute Antibody Ab02199-2.0, 1:200), Spike (Ms, BEI Resources NR-616, 1:200), ACE2 (ProteinTech 21115-1-AP, 1:200), TNNT2 (Abcam ab45932, 1:400), ACTN2 (Sigma A7732, 1:200), PECAM-1 (Santa Cruz sc1506, 1:50), GFP (Abcam ab13970, 1:200), MTCO2 (Abcam ab110258, 1:200), Hoechst 33342 (ThermoFisher 62249, 1:10,000). Images were acquired with a Zeiss Axio Observer Z.1 Spinning Disk Confocal (Carl Zeiss) or with an ImageXpress Micro Confocal High-Content Imaging System (Molecular Devices) and processed using ZenBlue and ImageJ.

### Histology

Paraffin sections of healthy and COVID-19 patient hearts were deparaffinized using xylene, re-hydrated through a decrease series of ethanol solutions (100%, 100%, 95%, 80%, 70%) and rinsed in PB1X. Hematoxylin and eosin staining was performed according manufacturer instructions and the slides were mounted with Cytoseal 60 (Richard-Allan Scientific) and glass coverslips. For immunofluorescence staining, epitopes were retrieved by immersing slides through 35 min incubation at 95° C. in citrate buffer (Vector Laboratories, pH 6) or Tris-EDTA buffer (Cellgro, pH 9). Slides were cooled for 20 min at RT and washed with PBS. Samples were permeabilized in 0.2% Triton X-100 (Sigma) in PBS by slide immersion and washed in PBS. Blocking was performed in 1.5% normal donkey serum (NDS; Jackson ImmunoResearch) and PBS solution for 1 h at RT. Primary and secondary antibody cocktails were diluted in blocking solution (Table 1). PBS washes were performed after primary (overnight, 4° C.) and secondary antibody (1 h, RT) incubations. Nuclei were stained with Hoechst and coverslips were mounted on slides using ProLong™ Gold Antifade Mountant. Samples were imaged on the Zeiss Axio Observer Z1.

TABLE 1B
Reagents

| Protein | Source | Catalog # | Dilution |
|---|---|---|---|
| ACE2 | ProteinTech | 21115-1-AP | 1:200 |
| ACTN2 | Sigma | A7732 | 1:200 |
| Alexa Fluor 488 Donkey | Invitrogen | | 1:400 |
| anti-mouse IgG | | | |
| Alexa Fluor 555 Donkey | Invitrogen | | 1:400 |
| anti-rabbit IgG | | | |
| Alexa Fluor 647 Donkey | Invitrogen | | 1:400 |
| anti-rabbit IgG | | | |
| Alexa Fluor 647 Donkey | Invitrogen | | 1:400 |
| anti-goat IgG | | | |
| Collagen IV | Millipore | AB769 | 1:100 |
| GFP | Abcam | ab13970 | 1:200 |
| Hoechst 33342 | Thermo Scientific | 62246 | 1:10000 |
| J2 | Absolute Antibody | Ab02199-2.0 | 1:200 |
| MTCO2 | Abcam | ab110258 | 1:200 |
| PECAM-1 | Santa Cruz | sc1506 | 1:50 |
| SARS Nucleoprotein | Thermo | MA1-7404 | 1:100 |
| Spike | BEI Resources | NR-616 | 1:200 |
| Troponin T | Abcam | ab45932 | 1:400 |

### RT-qPCR

Cultured cells were lysed with Qiagen buffer RLT (Qiagen, 79216) supplemented with 1% β-mercaptoethanol (Bio-Rad, 1610710) and RNA was isolated using the RNeasy Mini Kit (Qiagen 74104) or Quick-RNA MicroPrep (ThermoFisher, 50444593) and quantified using the NanoDrop 2000c (ThermoFisher). Viral load was measured by detection of the viral Nucleocapsid (N5) transcript through one-step quantitative real-time PCR, performed using PrimeTime Gene Expression Master Mix (Integrated DNA Technologies, 1055772) with primers and probes specific to N5 and RPP30 as in internal reference. RT-qPCR reactions were performed on a CFX384 (BioRad) and delta cycle threshold (ΔCt) was determined relative to RPP30 levels. Viral detection levels in pharmacologically treated samples were normalized to DMSO-treated controls.

TABLE 2
Primers

| Name | Direction | Sequence (5′-3′) |
|---|---|---|
| RNAse P | Forward | AGA TTT GGA CCT |
| PF_030_RP_F | (F) | GCG AGC G |
| | | (SEQ ID NO: 3) |
| RNAse P | Reverse | GAG CGG CTG TCT |
| PF_031_RP_R | (R) | CCA CAA GT |
| | | (SEQ ID NO: 4) |
| RNAse P | Forward | FAM-TTC TGA CCT |
| PF_032_RP_P | (probe) | GAA GGC TCT GCG |
| | | CG-BHQ1 |
| | | (SEQ ID NO: 5) |
| SARS-CoV-2 | Forward | AAATTTTGGGGACCAG |
| N gene | | GAAC |
| PF_039_nCoV_N5_F | | (SEQ ID NO: 6) |
| SARS-CoV-2 | Reverse | TGGCACCTGTGTAGGT |
| N gene | | CAAC |
| PF_040_nCoV_N5_R | | (SEQ ID NO: 7) |
| SARS-CoV-2 | Forward | ATGTCGCGCATTGGCA |
| N gene | (probe) | TGGA |
| PF_041_nCoV_N5_P | | (SEQ ID NO: 8) |
| SARS-CoV-2 | Forward | ACAGGTACGTTAATAG |
| E gene | | TTAATAGCGT |
| PF_042_nCoV_E_F | | (SEQ ID NO: 9) |
| SARS-CoV-2 | Reverse | ATATTGCAGCAGTACG |
| E gene | | CACACA |
| PF_043_nCoV_E_R | | (SEQ ID NO: 10) |
| SARS-CoV-2 | Forward | ACACTAGCCATCCTTA |
| E gene | (probe) | CTGCGCTTCG |
| PF_044_nCoV_E_P | | (SEQ ID NO: 11) |
| RNAse P | Forward | AGA TTT GGA CCT |
| PF_030_RP_F | | GCG AGC G |
| | | (SEQ ID NO: 12) |
| RNAse P | Reverse | GAG CGG CTG TCT |
| PF_031_RP_R | | CCA CAA GT |
| | | (SEQ ID NO: 13) |
| RNAse P | Forward | FAM-TTC TGA CCT |
| PF_032_RP_P | (probe) | GAA GGC TCT GCG |
| | | CG-BHQ1 |
| | | (SEQ ID NO: 14) |
| SARS-CoV-2 | Forward | AAATTTTGGGGACCAG |
| N gene | | GAAC |
| PF_039_nCoV_N5_F | | (SEQ ID NO: 15) |
| SARS-CoV-2 | Reverse | TGGCACCTGTGTAGGT |
| N gene | | CAAC |
| PF_040_nCoV_N5_R | | (SEQ ID NO: 16) |
| SARS-CoV-2 | Forward | ATGTCGCGCATTGGCA |
| N gene | (probe) | TGGA |
| PF_041_nCoV_N5_P | | (SEQ ID NO: 17) |

### RNA-Seq

For generating libraries for RNA-sequencing, RNA isolate quality was assessed with an Agilent Bioanalyzer 2100 on using the RNA Pico Kit (Agilent, 5067-1513). 10 ng of each RNA isolate was then prepared using the Takara SMARTer Stranded Total RNA-Seq Kit v2—Pico Input Mammalian (Takara, 634412). Transcripts were fragmented for 3.5 minutes and amplified for 12 cycles. Library concentrations were quantified with the Qubit dsDNA HS Assay Kit (Thermo Fisher, Q32851) and pooled for sequencing. Sequencing was performed on an Illumina NextSeq 550 system, using the NextSeq 500/550 High Output Kit v2.5 (150 Cycles) (Illumina, 20024907) to a depth of at least 10 million reads per sample.

### Bioinformatic Analyses

Samples were demultiplexed using bcl2fastq v2.20.0 and aligned to both GRCh38 and the SARS-CoV-2 reference sequence (NC_045512) using hisat2 v2.1.0 (see website at nature.com/articles/nmeth.3317). Aligned reads were converted to counts using featureCounts v1.6.2 (see website at pubmed.ncbi.nlm.nih.gov/24227677/). Cell-type clustering, gene loadings, and technical replication were assessed using the PCA and MDS projections implemented in scikit-learn v0.23.1 (see website at scikit-learn.org/stable/about.html#citing-scikit-leam). Differential expression analysis was performed using limma v3.44.3 with voom normalization (see website at genomebiology.biomedcentral.com/articles/10.1186/gb-2014-15-2-r29) and GO term enrichment analysis was performed using clusterProfiler v3.16.0 (see website at liebertpub.com/doi/10.1089/omi.2011.0118). Unbiased GO term selection was performed by non-negative matrix factorization using scikit-learn.

### TEM/CLEM

Cells grown on gridded 35 mm MatTek glass-bottom dishes (MatTek Corp., Ashland, MA, USA) were fixed in 2.5% glutaraldehyde and 2.5% paraformaldehyde in 0.1M sodium cacodylate buffer, pH 7.4 (EMS, Hatfield, PA, USA) following fluorescence imaging. Samples were rinsed 3×5 min at RT in 0.1M sodium cacodylate buffer, pH 7.2, and immersed in 1% osmium tetroxide with 1.6% potassium ferricyanide in 0.1M sodium cacodylate buffer for 30 minutes. Samples were rinsed (3×5 min, RT) in buffer and briefly washed with distilled water (1×1 min, RT) before sample were then subjected to an ascending ethanol gradient (7 min; 35%, 50%, 70%, 80%, 90%) followed by pure ethanol. Samples were progressively infiltrated (using ethanol as the solvent) with Epon resin (EMS, Hatfield, PA, USA) and polymerized at 60° C. for 24-48 hours. Care was taken to ensure only a thin amount of resin remained within the glass bottom dishes to enable the best possible chance for separation of the glass coverslip. Following polymerization, the glass coverslips were removed using ultra-thin Personna razor blades (EMS, Hatfield, PA, USA) and liquid nitrogen exposure as needed. The regions of interest, identified by the gridded alpha-numerical labeling, were carefully removed and mounted with cyanoacrylate glue for sectioning on a blank block. Serial thin sections (100 nm) were cut using a Leica UC 6 ultramicrotome (Leica, Wetzlar, Germany) from the surface of the block until approximately 4-5 microns in to ensure complete capture of the cell volumes. Section-ribbons were then collected sequentially onto formvar-coated 50 mesh copper grids. The grids were post-stained with 2% uranyl acetate followed by Reynold's lead citrate, for 5 min each. The sections were imaged using a Tecnai 12 120 kV TEM (FEI, Hillsboro, OR, USA), data were recorded using an UltraScan 1000 with Digital Micrograph 3 software (Gatan Inc., Pleasanton, CA, USA), and montaged datasets were collected with SerialEM (bio3d.colorado.edu/SerialEM) and reconstructed using IMOD eTOMO (bio3d.colorado.edu/imod).

### Example 2: Relative Susceptibility of Cardiac Cells to SARS-CoV-2 Infection

The relative infectability of different cardiac cell types had not previously been characterized for SARS-CoV-2, leading to ambiguity over the sources of cardiac damage and relevant therapeutic targets. The inventors determined the tropism of SARS-CoV-2 for different cardiac cell types by infecting cardiomyocytes (CMs), cardiac fibroblasts (CFs), endothelial cells (ECs), or a mix of all three with SARS-CoV-2 at a relatively low MOI (MOI=0.006).

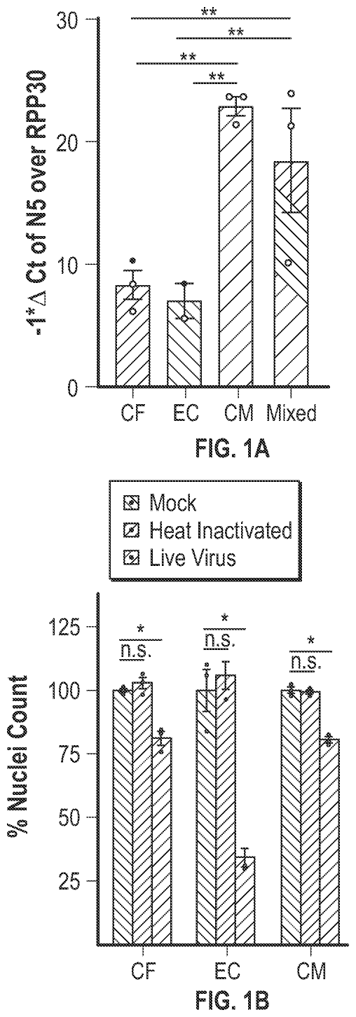

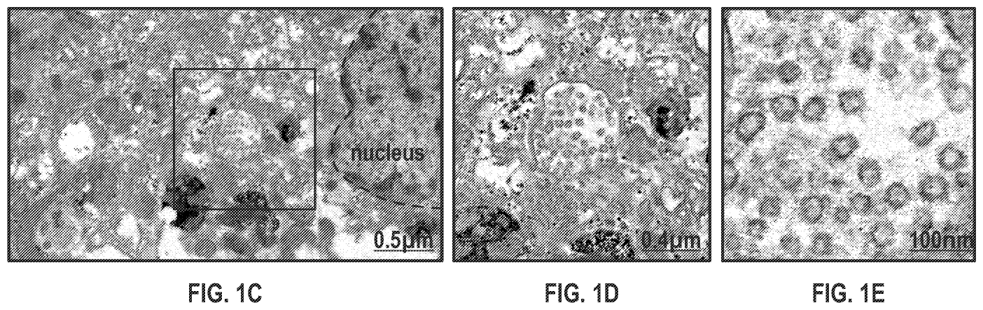

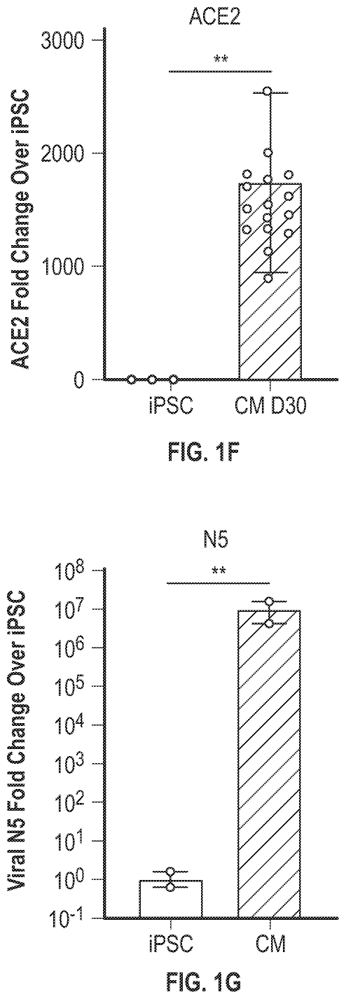

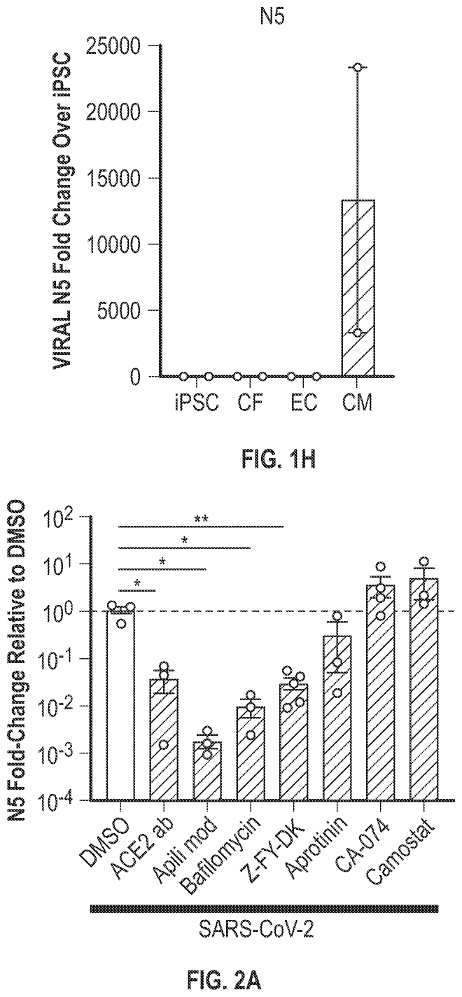

Viral infection load was measured by qPCR detection of the SARS-CoV-2 nucleocapsid transcript (N5) at 48 hours (FIG. 1A) or by immunostaining for double-stranded viral RNA (dsRNA) or Spike protein at 24, 48, and 72 hours (FIG. 1C-1E).

Viral replication measured in each cell type after 48 h largely correlated with corresponding ACE2 expression levels. Undifferentiated iPSCs were not infectable (FIG. 1F-1G). CFs and ECs also showed little to no viral N5 transcript detection (FIG. 1A, 1H), whereas CMs exhibited >10⁴ greater levels of viral RNA than any other cell type (FIG. 1A, 1C-1E, 1H). There was no significant difference in viral detection between CMs and mixed cultures, indicating that CMs are exclusively responsible for viral infection in the mixed cell condition that mimics native myocardial cellularity.

To further study if cardiac cells enable productive infection by SARS-CoV-2, plaque assays were performed on Vero cells from the supernatants of exposed cells that confirmed CFs, ECs, and iPSCs did not support productive infection, but CMs robustly produced new replication competent virions (FIG. 1H).

Immunostaining for replicating virus in the form of double-stranded viral RNA (dsRNA) or Spike protein further confirmed that infected CMs support viral replication. Positive dsRNA and Spike staining were only detected throughout infected CM cultures. Consistent with our qPCR results and plaque assays, CFs and ECs showed no dsRNA or Spike staining. However, all three cultures showed significant cytopathic effects after 48 hours of viral exposure, characterized by significant cell loss in all cell types (FIG. 1B-1E), fragmented cell bodies and dissociation from neighboring cells, with cytopathic effects most prevalent in CFs and particularly ECs (FIG. 1C-IE). Interestingly, despite cytopathic effects resulting from viral exposure without detectable infection, inoculation with heat-inactivated SARS-CoV-2 did not cause cell death or dissociation in any of the cell types assayed (FIG. 1B), suggesting the observed toxicity is due to live viral exposure.

Replication of (+)ssRNA viruses, including SARS-CoV and MERS-CoV, involves budding of double-membrane vesicles (DMVs) from the endoplasmic reticulum, with viral particle assembly occurring in the ER-Golgi intermediate compartment (ERGIC) cisternae (see website at biorxiv.org/content/10.1101/2020.06.23.167064v1). In CMs infected with SARS-CoV-2, dsRNA and Spike signals initially (24 h post infection) accumulated near the nucleus in small perinuclear puncta, closely matching the typical location of this ERGIC region, indicating potential active centers of replication. After 48 h post infection, an increase in the number of cells was observed with dsRNA signals throughout their cytoplasm, potentially correlating with breakdown of the ER-Golgi membrane as viral replication accelerates and the cell deteriorates, as evidenced by a decrease in sarcomeric integrity and intensity. By 72 h post infection, SARS-CoV-2 had spread throughout the culture and large swathes of the CMs had died, with the remaining cells displaying dispersed viral stain localization, dissociation from neighboring cells, and heavily reduced sarcomeric signal (FIG. 1C-1E).

Using transmission electron microscopy of infected CMs, the inventors readily identified the remnants of the ER-Golgi membranes and large vesicles in the proximity of the nucleus (FIG. 1C-1E). These vesicles, about 500-750 nanometers in diameter, contained multiple complete viral particles approximately 50-60 nm in diameter (FIG. 1D-1E), consistent with the dsRNA/Spike+ aggregates detected in infected CMs.

These results demonstrate that SARS-CoV-2 is able to readily infect, replicate in, and rapidly propagate through CMs.

### Example 3: Pharmacological Modulation of SARS-CoV-2 Cardiomyocytes Infection

Cardiomyocytes (CMs) were the only type of cell that proved infectable by SARS-CoV-2, from amongst the cell types tested (cardiomyocytes, cardiac fibroblasts, endothelial cells, and stem cells). This Example describes experiments for elucidating the mechanism of viral entry into CMs by using exogenous inhibition of CM factors.

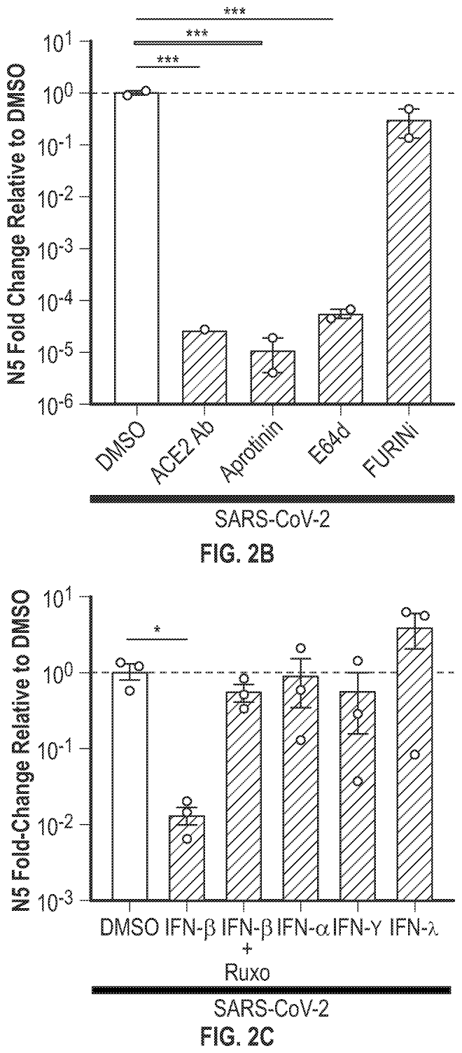

Cells pretreated with an ACE2 blocking antibody, cathepsin inhibitor E-64-D, or serine protease inhibitor aprotinin were able to significantly reduce the detection of viral transcripts in infected CMs (FIG. 2A-2B). Despite detection of FURIN in CMs, inhibition of FURIN (FURUNi) did not lead to a reduction in infection (FIG. 2B). Further probing revealed that cathepsin-L inhibition via Z-Phe-Tyr(tBu)-diazomethylketone (Z-FY-DK) was able to decrease viral detection in infected cells to about 10% of vehicle levels, but inhibition of cathepsin-B with CA-074 did not (FIG. 2A). In addition, the PIKfyve inhibitor apilimod and autolysosome acidification blocker bafilomycin also successfully reduced viral infection to ˜0.1% and 1% viral RNA detection compared to vehicle, respectively (FIG. 2A). In contrast, inhibition of TMPRSS2 with aprotinin or camostat mesylate did not significantly inhibit viral infection (FIG. 2A).

Taken altogether, these results strongly indicate that the SARS-CoV-2 virus employs the ACE2 receptor to bind to iPS-CMs and is able to utilize a cathepsin-L (CTSL)-dependent endolysosomal route, but not a cathepsin-B (CSTB)-dependent endolysosomal route, to infection without TMPRSS2/serine protease-mediated activation at the cellular membrane.

Based on the ability of SARS-CoV-2 to robustly infect and propagate through CMs, the inventors examined whether priming the innate immune response could effectively combat SARS-CoV-2 infection. CMs were primed with IFNα, IFNβ, IFNγ, or IFNλ, in addition to a combination of IFNs and a JAK/Stat inhibitor (ruxolitinib; ruxo) prior to infection. Only pre-exposure to IFNs was able to prevent infection, and this phenotype was reversed by JAK/Stat inhibition (FIG. 2C). Surprisingly, none of the other interferon exposures were able to prevent infection (FIG. 2C). Single-cell RNA-sequencing data indicated that CMs express undetectable levels of IFNβ, perhaps indicating that their high infectivity may be due to an intrinsic inability to appropriately trigger a sufficient immune response to combat viral infection.

### Example 4: Transcriptomic Response to SARS-CoV-2 Exposure

This Example describes experiments for evaluating the transcriptional response of cardiac cells exposed to SARS-CoV-2, and in particular to identify differences in the level of immune suppression or cytokine activation across different levels of viral load. The experiments involved RNA-sequencing of infected and mock-treated CFs, ECs, and iPSCs at a MOI of 0.006, or a range of MOIs (0.001, 0.01, and 0.1) for CMs.

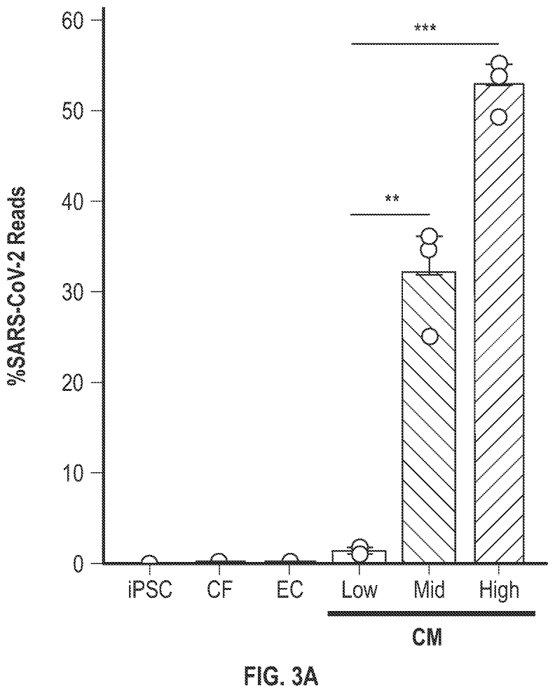

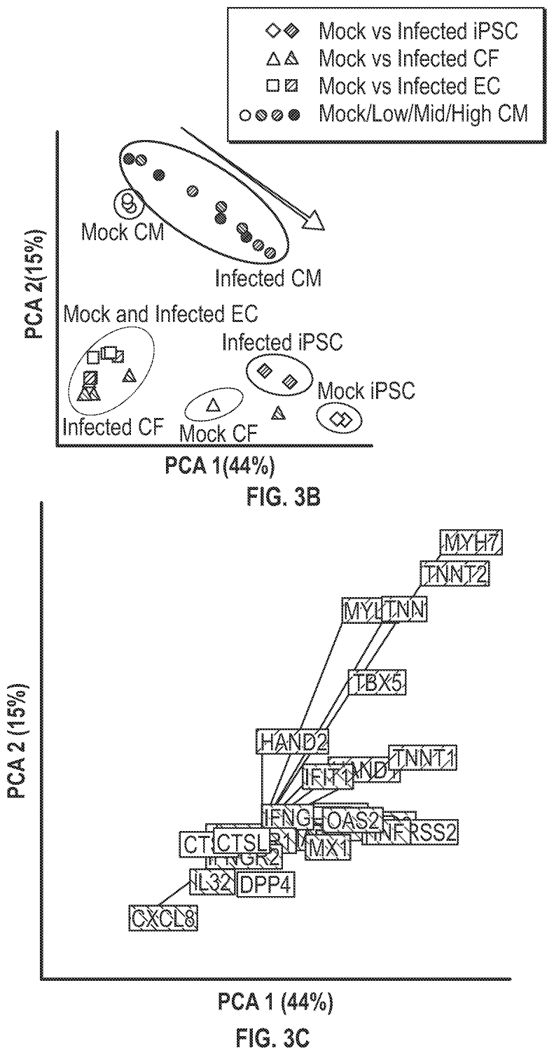

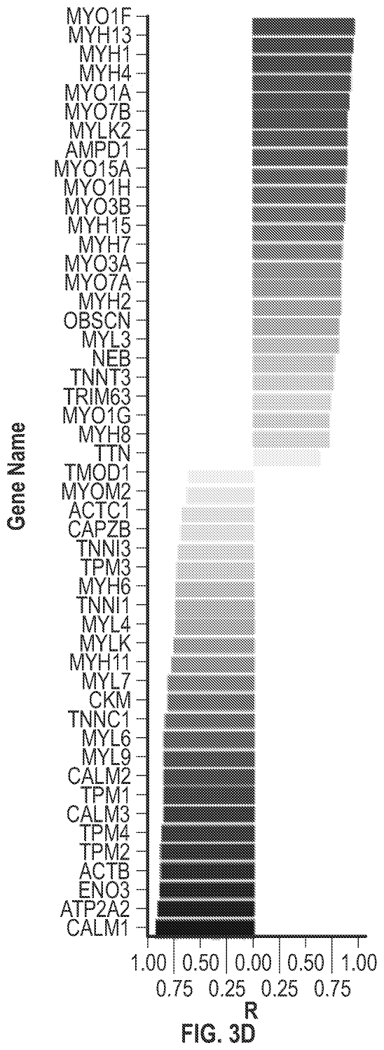

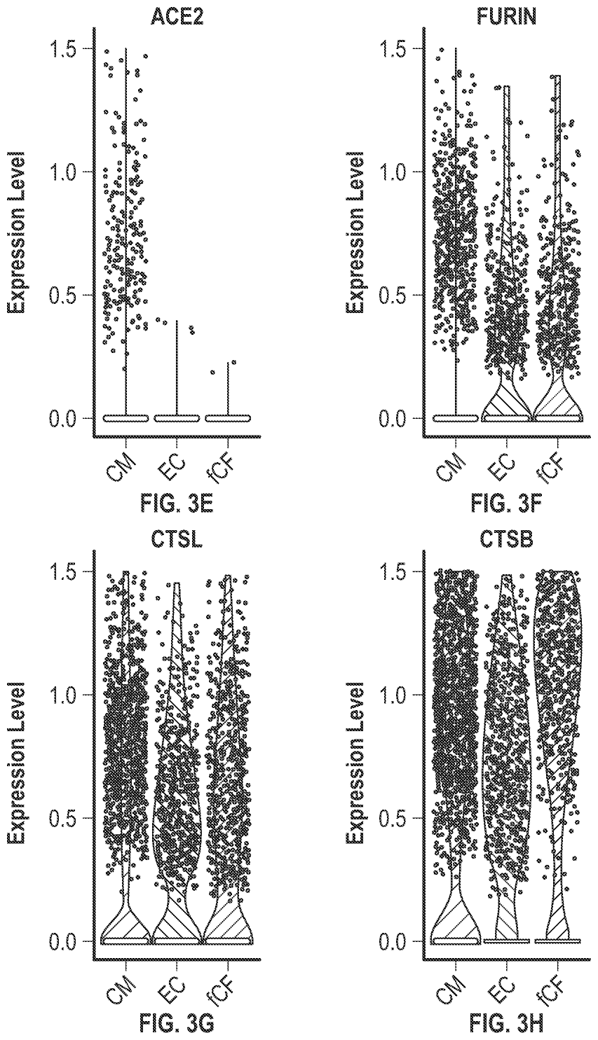

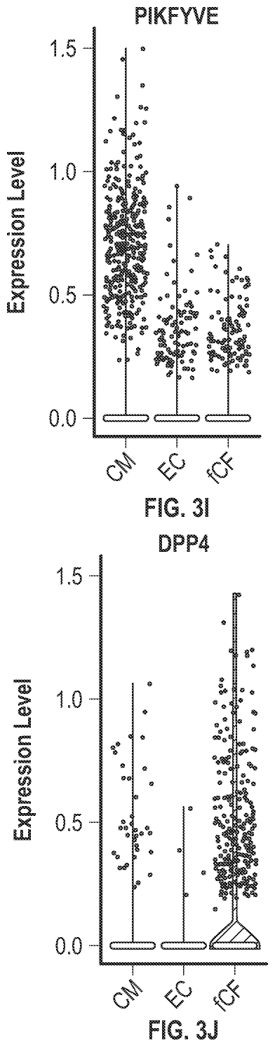

Sequencing recovered a high proportion of SARS-CoV-2 transcripts in an MOI and cell-type dependent fashion (FIG. 3A), with CMs at the highest MOI reaching >50% SARS-CoV-2 recovered reads (FIG. 3A). Principal component analysis (PCA) of the biological conditions revealed the expected clustering primarily based on cell type, with CFs and ECs clustering near together and CMs and iPSCs clustering separately (FIG. 3B). Loading plots of the principal components complemented this interpretation: the genes determining the spectrum of variation between CMs and CF/ECs were associated with CMs (MYH7, MYH6, TNNT2) at one pole (FIG. 3C) and anti-correlated with CF/EC specific genes at the other (FN1, COL1A2, TFPI2, MME). Notably, the distance between mock CMs and the furthest infected CMs was slightly further than the distance between CMs and CFs or ECs, indicating that viral infection altered cellular expression profiles at least as strongly as cellular identity. Along this axis, however, the inventors also observed that the level of transcriptional disruption correlated poorly with MOI across all CM samples, potentially due to natural stochasticity in the kinetics of infection. Regrouping conditions by the level of transcriptional disruption showed transcriptional trends resulting from viral exposure more clearly.

However, the significant distance between infected and mock conditions indicates that viral infection impacted the variation in expression profiles at least as strongly as the differences in cell type. Individual samples within the low, middle, and high MOI conditions correlated poorly with the degree of transcriptional disruption observed, potentially due to natural stochasticity in the kinetics of infection.

Regrouping conditions by the level of transcriptional disruption allowed transcriptional trends to be deduced as a function of viral impact. Loading plots of the principal components indicated that the main axis of variation aligned along a CM, CF/EC spectrum with CM specific genes (MYH7, MYH6, TNNT2) at one pole (FIG. 3C), anti-correlated with CF/EC specific genes (FN1, COL1A2).

Analysis of differential regulation of genes involved in inflammation and innate immunity for infected CFs, ECs, and CMs agree with the observed infectivity of CMs. Infected CFs and ECs have a depressed cytokine response compared to all three levels of disrupted CMs, which are enriched for genes involved in cytokine production and T-cell activation (OAS2, MX1, IFIT1, IL1B, IL6, TNF) (FIG. 3D) in addition to olfactory receptor (OR) genes, the ectopic expression of which may reflect a stress response (see websites at link.springer.com/chapter/10.1007/978-3-319-26932-0_33; www.nature.com/articles/s41573-018-0002-3?WT.feed_name=subjects_neuroscience).

Interestingly, the inventors noted that CMs at each MOI showed very clear dysregulation of genes involved in contractile machinery and proteasome homeostasis. All MOI conditions tested showed very clear dysregulation of genes involved in contractile machinery and proteasome homeostasis. In particular, sarcomeric structural proteins, myosin light chains, and proteasome kinases and chaperones were strongly downregulated, and most myosin heavy chains were significantly upregulated (FIG. 3D), indicating a potential effect of SARS-CoV-2 infection in the contractile and structural integrity of CMs.

In light of observations that impairment of cardiac function can occur even in mild cases of COVID-19 (which were mimicked by low MOIs), these results illustrate that SARS-CoV-2 may have unique interactions with structural features of CMs that can potentially cause cardiac dysfunction. Deeper analyses of the individual genes driving the GO terms revealed significant downregulation of mitochondrial metabolism networks, decreased regulation of protein degradation, and loss of genes associated with sarcomere formation and maintenance.

### Example 5: Differential Expression of Viral Entry Factors in Cardiac Cells

Historical single-cell RNA-Seq data was first analyzed to determine the expression of putative viral entry host factors in CMs, ECs, and primary cardiac fibroblasts (see website at biorxiv.org/content/10.1101/2020.07.06.190504v1).

The primary SARS-CoV-2 receptor, ACE2, was detected at low levels in all cells, but ACE2 displayed greater than 10-fold higher expression in cardiomyocytes than in cardiac fibroblasts or endothelial cells, indicating that cardiomyocytes are more susceptible to infection than other cardiac cell types (FIG. 3E). Of the proteases thought to cleave the viral Spike protein to prime SARS-CoV-2 entry, TMPRSS2 was not detected in any cell types, but FURIN was ubiquitously expressed (FIG. 3F). It has also been proposed that SARS-CoV-2 can infect cells via endocytosis (see website at nature.com/articles/s41467-020-15562-9), similar to SARS-CoV. Endosomal entry factors for SARS-CoV include cathepsin-L (CTSL), cathepsin-B (CTSB), and the endosomal kinase PIKfyve (see website at pnas.org/content/102/33/11876.short). Protein structural similarity studies predict that these factors can also act on SARS-CoV-2 (see website at mdpi.com/2076-0817/9/3/186), and all three were expressed in all the examined cell types, with elevated PIKfyve in CMs (FIG. 3G-3I. In addition, DPP4, the surface protease used by the closely related MERS-CoV (see website at nature.com/articles/cr201392) and speculated to facilitate SARS-CoV-2 invasion (see website at ncbi.nlm.nih.gov/pmc/articles/PMC7103712/), was also detected, though at higher levels in primary cardiac fibroblasts (FIG. 3J).

These data support the viability of SARS-CoV-2 infection of cardiac cells via an ACE2-endocytosis axis.

To validate expression of the ACE2 receptor in CMs, the inventors directly examined ACE2 transcript and protein expression. While ACE2 transcripts were undetected in iPSCs by qPCR, differentiated and purified CMs exhibited robust expression (FIG. 1F). Heterotypic tissues comprising CMs and iPS-derived stromal non-myocytes were also examined, and strong expression was observed of ACE2 protein in cardiac muscle troponin T (cTnT)+ CMs while low to no expression in the surrounding cTnT-non-myocytes.

These results demonstrate that CMs are susceptible to SARS-CoV-2 infection.

### Example 6: SARS-CoV-2 Infection Disrupts Multiple Intracellular CM Features

As described in this Example, motivated by the discovery of disruptions to various structural and contractile genes in our transcriptomic data, the inventors performed high content imaging of CMs following SARS-CoV-2 infection.

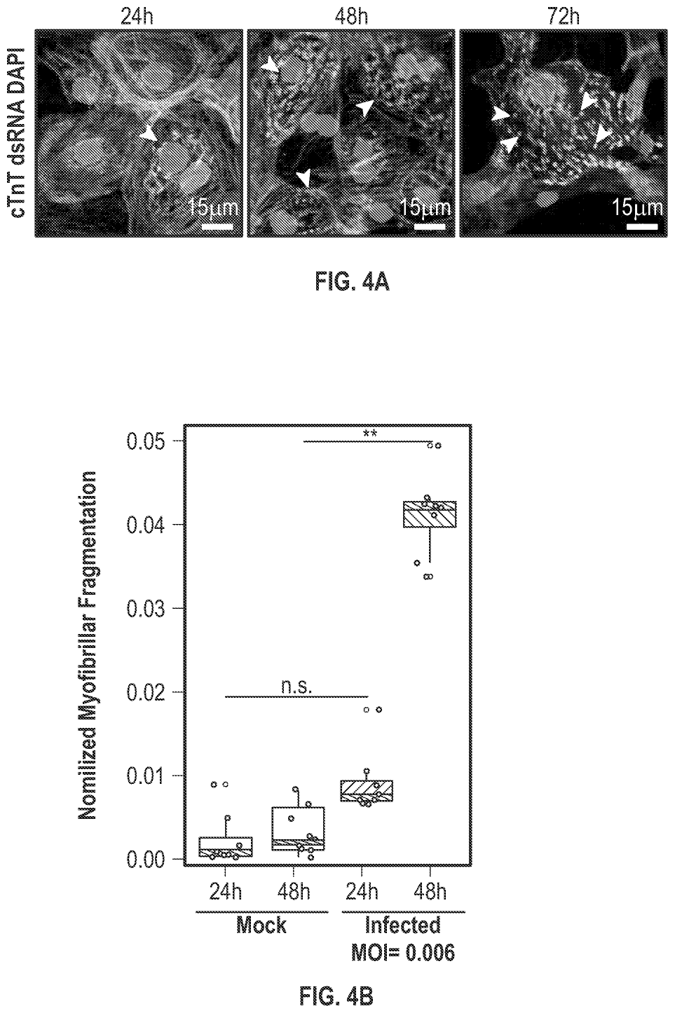

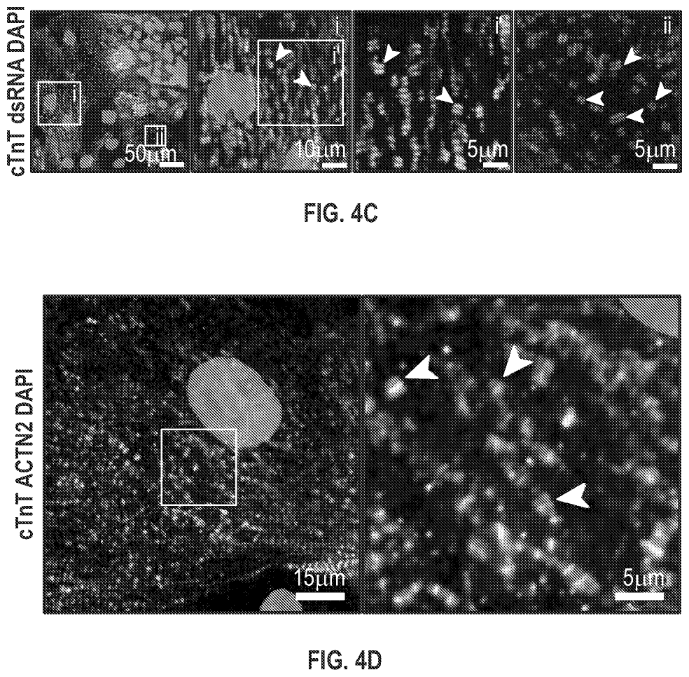

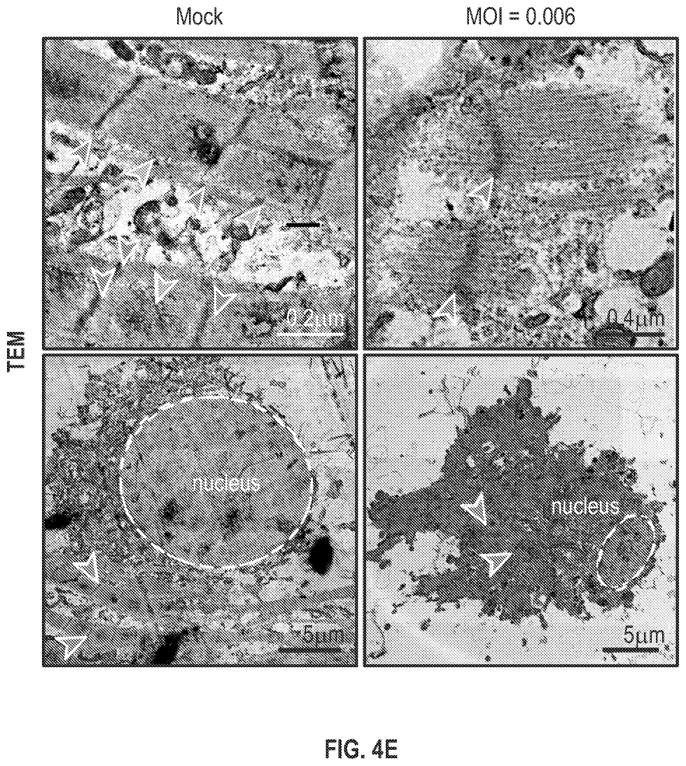

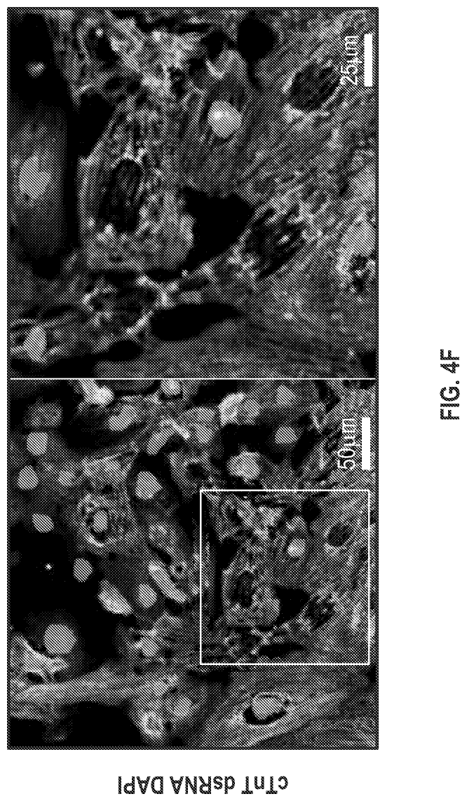

A number of abnormal structural features were immediately observed in many of the infected CMs that were not seen in parallel mock samples. Widespread myofibrillar disruption throughout the cytoplasm was the most common feature observed, which manifested as a unique pattern of very specific and periodic cleavage of myofibrils into individual sarcomeric units of identical size but without any alignment (FIG. 4A). Evidence of sarcomeric fragmentation was generally identified as early as 24 hours after infection, but was more widespread and common after 48 hours, and also observed in many of the CMs that remained after 72 hours. At a single time point 48 h post infection, up to 20% of cells exposed to virus displayed similar phenotypes of this rapid fragmentation (FIG. 4B), indicating this is a pervasive and continuous phenomenon. Curiously, myofibrillar fragmentation was more prevalent in bystander CMs that lacked signs of active viral infection (as per viral dsRNA staining), while cells positive for dsRNA rarely showed signs of myofibrillar fragmentation. The inventors found an inverse correlation (p-value<0.01) between the number of viral RNA positive cells in a well and the number of cells presenting sarcomere fragmentation (FIG. 4A-4C).

Since transcriptomic profiling data indicated viral infection altered the proteasome system (FIG. 3), CMs were exposed to the proteasome inhibitor bortezomib and observed that only high doses of bortezomib (but not the well-known cardiotoxic drug doxorubicin) induced myofibril fragmentations in CMs. However, bortezomib treatment induced fragmentation much more infrequently and less severely than SARS-CoV-2 and was generally accompanied by diffuse cTnT staining throughout the cell cytoplasm.

Altogether, these results indicate that the observed fragmentation of the sarcomere is dependent on SARS-CoV-2 infection of neighboring CMs. Reducing productive infection of CMs by means of IFN-β pre-treatment or E64D treatment did not reduce the incidence of myofibrillar disruption. However, ACE2 blocking did reduce the incident of myofibrillar disruption, potentially indicating an immediate response to viral exposure to the cell surface.

Co-staining SARS-CoV-2-exposed CMs with cTnT and the Z-disk marker α-actinin 2 revealed the myofibrillar fragments observed upon SARS-CoV-2 exposure consisted of two cTnT-positive bands flanking a single α-actinin 2 band, indicating cleavage at the M-line or a separation of thick and thin filaments (FIG. 4D). To examine sarcomeric fragmentation in greater detail, the inventors employed TEM imaging of SARS-CoV-2 infected and mock-treated cardiomyocytes. While intact sarcomeres were clearly identifiable with a classic dark Z-disk, light I-band, and dark A-band, single fragmented sarcomeres displayed an extended I-band and complete absence of the A-band (FIG. 4E), suggesting a mechanism by which thick filaments are liberated from sarcomere subunits. The intracellular network of mitochondria in CMs exposed to SARS-CoV-2 also appeared to be disrupted relative to normal mitochondrial organization.

In addition, the inventors observed that CMs with intact or moderately disrupted myofibrils often appeared to lack nuclear DNA staining (FIG. 4F). This phenomenon was observed most frequently in localized patches, with numerous cells lacking dsRNA staining along with stark nuclear absence (FIG. 4F).

### Example 7: Intracellular Disruption in Myocardium of COVID-19 Patients

Based on the in vitro findings, the inventors sought to identify whether similar features were contributing to COVID-19 myocardial damage in vivo. The sarcomere fragmentation observed in COVID-19 patients appears to present some extreme features even compared to in vitro system.

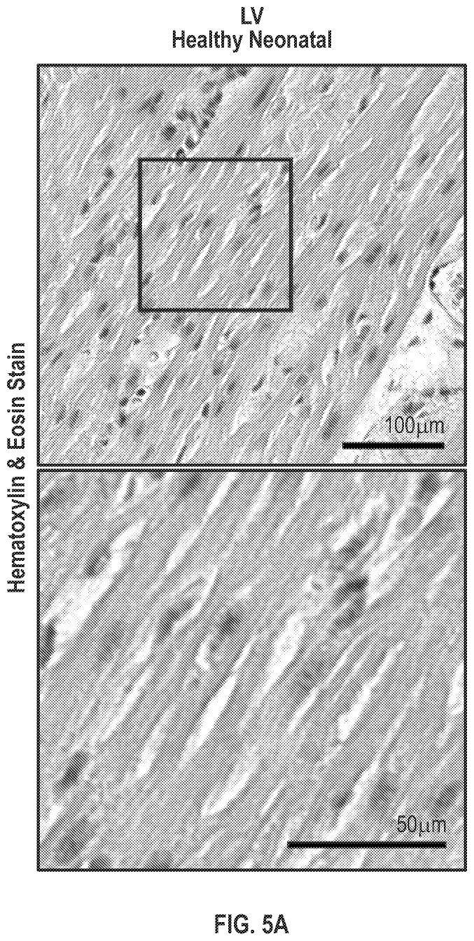

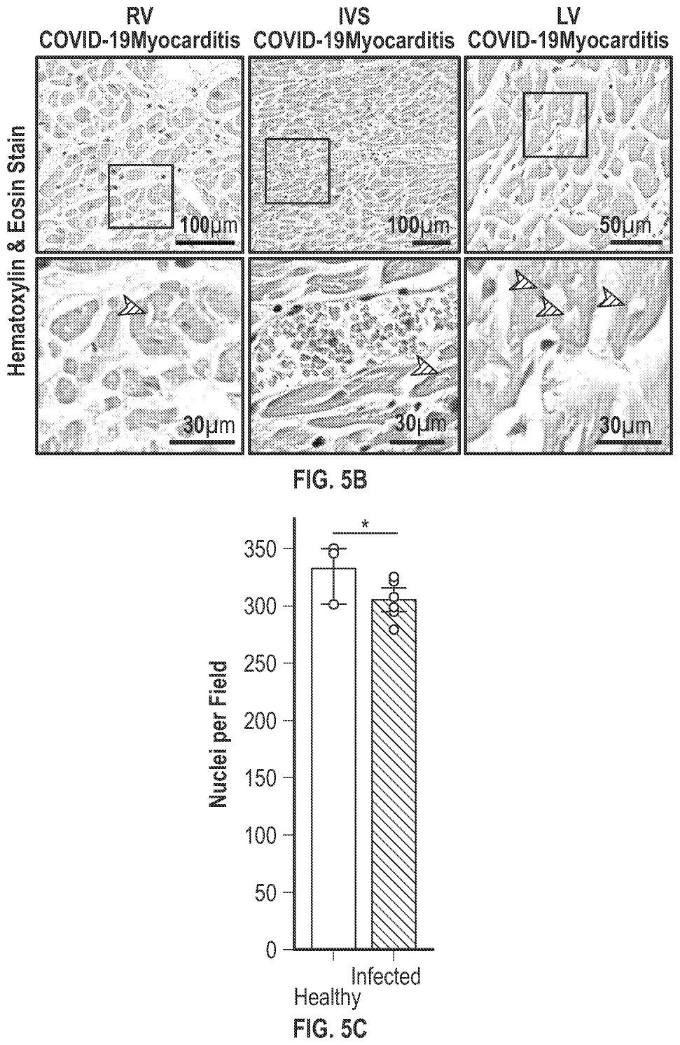

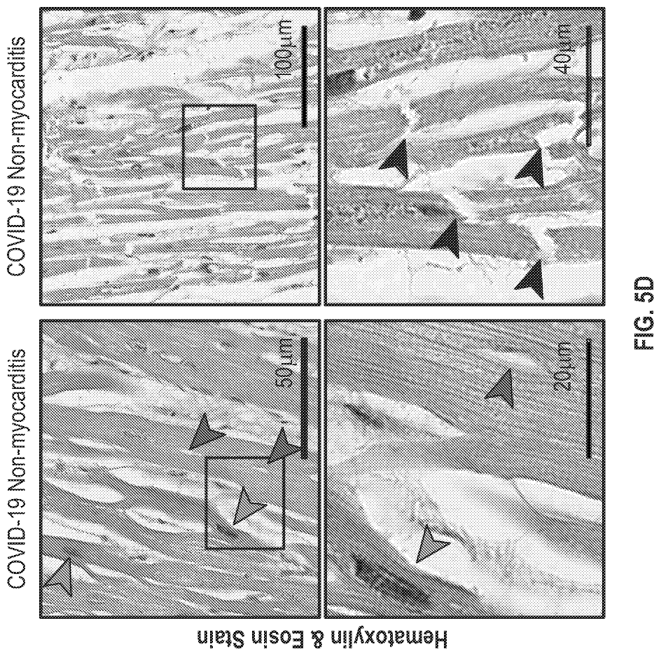

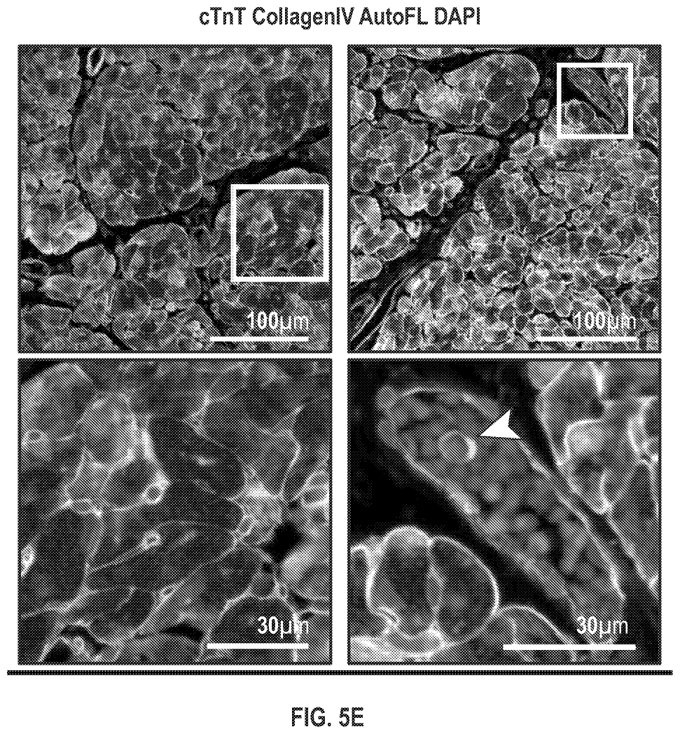

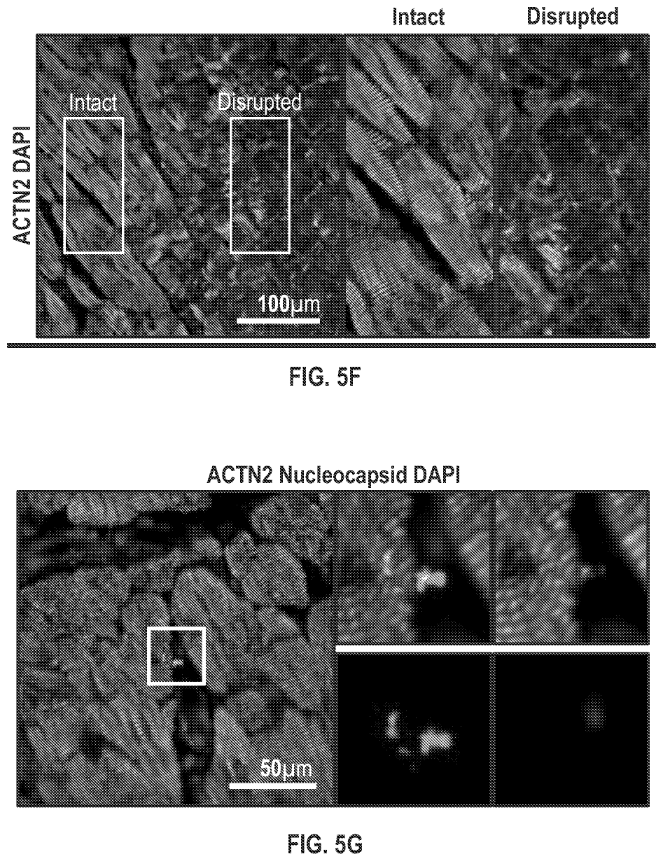

Patient specimens were obtained from four COVID-19 positive patients—one diagnosed with viral myocarditis. Compared to healthy myocardial tissue (FIG. 5A), significant histological alterations were observed of the myocardium in the COVID-19 myocarditis case (FIG. 5B), in addition to moderate levels of immune infiltration. Mononuclear cells that appeared to be immune cells were detected, as well as nuclei with loss of nuclear material. Intercalated disks between cardiomyocytes were broken.

The tissues from the COVID-19 myocarditis case exhibited signs of edema with increased spacing between adjacent cardiomyocytes (FIG. 5B) and highly uneven staining for cardiac troponin-T, indicating sarcomere disruption (FIG. 5B, 5E) and there was evidence of troponin-T positive cells in the blood vessels, indicating phagocytosis of compromised myocytes (FIG. 5B, 5E). Some of the observed cardiomyocytes lacked hematoxylin staining for nuclei, showing that the in vitro phenotype of nuclear loss was also observed in patients infected with COVID-19 (FIG. 5B).

In COVID-19 infected patients that were not diagnosed with myocarditis (FIG. 5D), clear evidence was observed of nuclear loss (FIG. 5D) as well as evidence of myocyte compaction (FIG. 5D), and large regions exhibiting significant disruption of intercalated disk connections between cardiomyocytes (FIG. 5D). Strikingly, immunohistochemical labeling of the myofibrils revealed regions of extreme myofibrillar anomalies. Patients without diagnoses of myocarditis present large regions of myofibrils (ACTN2+) within cardiomyocytes that were entirely missing or collapsed (FIG. 5D).

The results described herein demonstrate that the in vitro phenotypes are able to predict previously unobserved disruptions in myocardium. Therefore, the in vitro methods described herein can be used to dissect the mechanisms of COVID-19 cardiovascular injury and identify agents that reduce or inhibit such injury.

### REFERENCES

Honko, A. N.; Storm, N.; Bean, D. J.; Henao Vasquez, J.; Downs, S. N.; Griffiths, A. Rapid Quantification and Neutralization Assays for Novel Coronavirus SARS-CoV-2 Using Avicel RC-591 Semi-Solid Overlay. Preprints 2020, 2020050264 (doi: 10.20944/preprints202005.0264.v1)
Lian, X. et al. Robust cardiomyocyte differentiation from human pluripotent stem cells via temporal modulation of canonical Wnt signaling. PNAS (2012) doi:10.1073/pnas.1200250109.
Tohyama, S. et al. Distinct Metabolic Flow Enables Large-Scale Purification of Mouse and Human Pluripotent Stem Cell-Derived Cardiomyocytes. Cell Stem Cell 12, 127-137 (2013).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A method comprising: incubating one or more test agents with cardiomyocytes in the presence of SARS-CoV-2 virus; and identifying any of the one or more test agents that reduce any of myofibrillar disruption, sarcomeric fragmentation, nuclear material, nuclear staining, enucleation, cardiac troponin solute levels, herniated mitochondria, apoptotic mitochondria, or a combination thereof in the cardiomyocytes compared to a control assay comprising with cardiomyocytes in the presence of SARS-CoV-2 virus without the test agent(s).
2. The method of statement 1, wherein the SARS-CoV-2 virus is present at a multiplicity of infection at one or more SARS-CoV-2 virion particle per about 1000 cardiomyocyte cells; or at two or more SARS-CoV-2 virion particles per about 1000 cardiomyocyte cells; or at three or more SARS-CoV-2 virion particles per about 1000 cardiomyocyte cells; or at five or more SARS-CoV-2 virion particles per about 1000 cardiomyocyte cells; or at ten or more SARS-CoV-2 virion particles per about 1000 cardiomyocyte cells.
3. The method of statement 1 or 2, wherein the SARS-CoV-2 virion particles infect cardiomyocytes, but do not infect cardiac fibroblasts, endothelial cells, or stem cells.
4. The method of statement 1, 2, or 3, wherein the cardiomyocytes are generated from induced pluripotent stem cells.
5. The method of statement 1-3 or 4, wherein the SARS-CoV-2 virion particles do not infect induced pluripotent stem cells.
6. The method of any one of statements 1-5, wherein the cardiomyocytes are from a subject without a cardiac condition or a cardiac disease.
7. The method of any one of statements 1-5, wherein the cardiomyocytes are mutant cardiomyocytes.
8. The method of any one of statements 6 or 7, wherein the cardiac condition or a cardiac disease comprises a genetic mutation or a disease correlated with a genetic mutation.
9. The method of any one of statements 1-7 or 8, wherein the cardiomyocytes are from a subject with a cardiac condition or a cardiac disease.
10. The method of any one of statement 6-9, wherein the mutant cardiomyocytes, the cardiac condition, or the cardiac disease leads to or contributes to impairments in contractility, impairments in ability to relax (e.g., diastolic dysfunction), abnormal or improper functioning of the heart's valves, diseases of the heart muscle (e.g., cardiomyopathies), diseases such as angina pectoris, myocardial ischemia, infarction characterized by inadequate blood supply to the heart muscle, infiltrative diseases such as amyloidosis and hemochromatosis, global or regional hypertrophy (e.g., as may occur in some kinds of cardiomyopathy or systemic hypertension), abnormal communications between chambers of the heart, or a combination thereof in the subject.
11. The method of any one of statement 6-10, wherein the mutant cardiomyocytes, the cardiac condition, or the cardiac disease can lead to or can contribute to a disease or dysfunction of the myocardium (heart muscle) in which a heart is abnormally enlarged, thickened and/or stiffened in a subject.
12. The method of any one of statements 6-11, wherein the mutant cardiomyocytes, the cardiac condition, or the cardiac disease can lead to or can contribute to ischemic cardiomyopathy, coronary artery disease, non-ischemic cardiomyopathy, dilated cardiomyopathy, hypertrophic cardiomyopathy, restrictive cardiomyopathy, infiltrative cardiomyopathy, congestive heart failure, myocardial infarction, cardiac ischemia, myocarditis, arrhythmia, or a combination thereof in a subject.
13. The method of any one of statements 6-12, wherein the mutant cardiomyocytes, cardiac condition or a cardiac disease leads to or contributes to myocarditis, Duchenne muscular dystrophy or Emery Dreiffuss dilated cardiomyopathy in a subject.
14. The method of any one of statements 1-13, comprising identifying (e.g., by Hoechst or hematoxylin staining) any of the one or more test agents that reduce cardiomyocyte enucleation compared to the control assay.
15. The method of any one of statements 1-14, comprising identifying any of the one or more test agents that reduce titin protein cleavage compared to the control assay; or comprising identifying any of the one or more test agents that reduce M-band titin cleavage compared to the control assay.
16. The method of any one of statements 1-15, wherein one or more of the test agents is a small molecule, an antibody, a nucleic acid, a carbohydrate, a protein, or a combination thereof.
17. The method of any one of statements 1-16, wherein the one or more test agents block ACE2, inhibit cathepsin, or inhibit serine proteases.
18. The method of any one of statements 1-17, further comprising manufacturing one or more of the test agents that reduce myofibrillar disruption, sarcomeric fragmentation, nuclear staining, enucleation, cardiac troponin solute levels, or a combination thereof.
19. The method of any one of statements 1-18, further comprising administering to an animal one or more of the test agents that reduce myofibrillar disruption, sarcomeric fragmentation, nuclear staining, enucleation, cardiac troponin solute levels, or a combination thereof.
20. One or more compounds identified by the method of any one of statements 1-19.
21. The one or more compounds of statement 20 formulated into a composition.
22. The one or more compounds of statement 20 or 21, comprising an ACE2 blocking agent, a cathepsin inhibitor, or a serine protease inhibitor.
23. The one or more compounds of statement 20, 21 or 22, comprising an ACE2 blocking antibody, cathepsin inhibitor E-64-D, or aprotinin.
24. A method comprising administering to a subject one or more of the compounds of statement 20-22 or 23.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed:

1. A method comprising: incubating one or more test agents with cardiomyocytes in the presence of SARS-CoV-2 virus; and identifying any of the one or more test agents that reduce myofibrillar disruption, sarcomeric fragmentation, nuclear staining, enucleation, cardiac troponin solute levels, or a combination thereof in the cardiomyocytes compared to a control assay comprising with cardiomyocytes in the presence of SARS-CoV-2 virus without the test agent(s).

2. The method of claim 1, wherein the SARS-CoV-2 virus is present at a multiplicity of infection of one or more SARS-CoV-2 virion particle per about 1000 cardiomyocyte cells; or of two or more SARS-CoV-2 virion particles per about 1000 cardiomyocyte cells; or of three or more SARS-CoV-2 virion particles per about 1000 cardiomyocyte cells; or of five or more SARS-CoV-2 virion particles per about 1000 cardiomyocyte cells; or of ten or more SARS-CoV-2 virion particles per about 1000 cardiomyocyte cells.

3. The method of claim 1, wherein the SARS-CoV-2 virion particles infect cardiomyocytes, but do not infect cardiac fibroblasts, endothelial cells, or stein cells.

4. The method of claim 1, wherein the cardiomyocytes are generated from induced pluripotent stem cells.

5. The method of claim 1, wherein the SARS-CoV-2 virion particles do not infect induced pluripotent stem cells.

6. The method of claim 1, wherein the cardiomyocytes are mutant cardiomyocytes.

7. The method of claim 1, wherein the cardiomyocytes are from a subject without a cardiac condition or a cardiac disease.

8. The method of claim 7, wherein the cardiac condition or a cardiac disease comprises a genetic mutation or a disease correlated with a genetic mutation.

9. The method of claim 1, wherein the cardiomyocytes comprise a mutation or genetic variation that leads to or contributes to impairments in contractility, impairments in ability to relax, diastolic dysfunction, abnormal or improper functioning of the heart's valves, cardiomyopathies, angina pectoris, myocardial ischemia, infarction, hypertension, inadequate blood supply to heart muscle, amyloidosis, hemochromatosis, global hypertrophy, regional hypertrophy, abnormal communications between heart chambers, or a combination thereof in a subject.

10. The method of claim 1, wherein the cardiomyocytes comprise a mutation or genetic variation that leads to or contributes to an abnormally enlarged, thickened heart, an abnormally stiffened heart, or a combination thereof in a subject.

11. The method of claim 1, wherein the cardiomyocytes comprise a mutation or genetic variation that leads to or contributes to ischemic cardiomyopathy, coronary artery disease, non-ischemic cardiomyopathy, dilated cardiomyopathy, hypertrophic cardiomyopathy, restrictive cardiomyopathy, infiltrative cardiomyopathy, congestive heart failure, myocardial infarction, cardiac ischemia, myocarditis, arrhythmia, or a combination thereof in a subject.

12. The method of claim 1, wherein the cardiomyocytes comprise a mutation or genetic variation that leads to or contributes to myocarditis, Duchenne muscular dystrophy or Emery Dreiffuss dilated cardiomyopathy in a subject.

13. The method of claim 1, comprising Hoechst and/or hematoxylin staining.

14. The method of claim 1, comprising identifying one or more test agents that reduce cardiomyocyte enucleation compared to the control assay.

15. The method of claim 1, comprising identifying one or more test agents that reduce titin cleavage compared to the control assay.

16. The method of claim 1, comprising identifying any of the one or more test agents that reduce M-band titin cleavage compared to the control assay.

17. The method of claim 1, wherein one or more of the test agents is a small molecule, an antibody, a nucleic acid, a carbohydrate, a protein, or a combination thereof.

18. The method of claim 1, wherein the one or more test agents block ACE2, inhibit cathepsin, or inhibit serine proteases.

19. The method of claim 1, further comprising manufacturing one or more of the test agents that reduce myofibrillar disruption, sarcomeric fragmentation, nuclear staining, enucleation, cardiac troponin solute levels, or a combination thereof.

20. The method claim 1, further comprising administering to an animal or subject one or more of the test agents that reduce myofibrillar disruption, sarcomeric fragmentation, nuclear staining, enucleation, cardiac troponin solute levels, or a combination thereof.

21. The method of claim 1, further comprising formulating one or more test agents into a composition.

22. The method of claim 21, further comprising formulating the composition to comprise ACE2 blocking agent, a cathepsin inhibitor, or a serine protease inhibitor.

23. The method of claim 21, comprising administering the composition to an animal or subject.


SEQUENCE LISTING (17 sequences)

SEQ ID NO: 1 - protein, 27118 aa, Homo sapiens
MTTQAPTFTQPLQSVVVLEGSTATFEAHISGFPVPEVSWFRDGQVISTSTLPGVQISFSDGRAKLTIPAVTKANSGRYSLKATNGSGQATSTAELLVKAETAPPNFVQRLQSMTVRQGSQVRLQVRVTGIPTPVVKFYRDGAEIQSSLDFQISQEGDLYSLLIAEAYPEDSGTYSVNATNSVGRATSTAELLVQGEEEVPAKKTKTIVSTAQISESRQTRIEKKIEAHFDARSIATVEMVIDGAAGQQLPHKTPPRIPPKPKSRSPTPPSIAAKAQLARQQSPSPIRHSPSPVRHVRAPTPSPVRSVSPAARISTSPIRSVRSPLLMRKTQASTVATGPEVPPPWKQEGYVASSSEAEMRETTLTTSTQIRTEERWEGRYGVQEQVTISGAAGAAASVSASASYAAEAVATGAKEVKQDADKSAAVATVVAAVDMARVREPVISAVEQTAQRTTTTAVHIQPAQEQVRKEAEKTAVTKVVVAADKAKEQELKSRTKEVITTKQEQMHVTHEQIRKETEKTFVPKVVISAAKAKEQETRISEEITKKQKQVTQEAIMKETRKTVVPKVIVATPKVKEQDLVSRGREGITTKREQVQITQEKMRKEAEKTALSTIAVATAKAKEQETILRTRETMATRQEQIQVTHGKVDVGKKAEAVATVVAAVDQARVREPREPGHLEESYAQQTTLEYGYKERISAAKVAEPPQRPASEPHVVPKAVKPRVIQAPSETHIKTTDQKGMHISSQIKKTTDLTTERLVHVDKRPRTASPHFTVSKISVPKTEHGYEASIAGSAIATLQKELSATSSAQKITKSVKAPTVKPSETRVRAEPTPLPQFPFADTPDTYKSEAGVEVKKEVGVSITGTTVREERFEVLHGREAKVTETARVPAPVEIPVTPPTLVSGLKNVTVIEGESVTLECHISGYPSPTVTWYREDYQIESSIDFQITFQSGIARLMIREAFAEDSGRFTCSAVNEAGTVSTSCYLAVQVSEEFEKETTAVTEKFTTEEKRFVESRDVVMTDTSLTEEQAGPGEPAAPYFITKPVVQKLVEGGSVVFGCQVGGNPKPHVYWKKSGVPLTTGYRYKVSYNKQTGECKLVISMTFADDAGEYTIVVRNKHGETSASASLLEEADYELLMKSQQEMLYQTQVTAFVQEPKVGETAPGFVYSEYEKEYEKEQALIRKKMAKDTVVVRTYVEDQEFHISSFEERLIKEIEYRIIKTTLEELLEEDGEEKMAVDISESEAVESGFDSRIKNYRILEGMGVTFHCKMSGYPLPKIAWYKDGKRIKHGERYQMDFLQDGRASLRIPVVLPEDEGIYTAFASNIKGNAICSGKLYVEPAAPLGAPTYIPTLEPVSRIRSLSPRSVSRSPIRMSPARMSPARMSPARMSPARMSPGRRLEETDESQLERLYKPVFVLKPVSFKCLEGQTARFDLKVVGRPMPETFWFHDGQQIVNDYTHKVVIKEDGTQSLIIVPATPSDSGEWTVVAQNRAGRSSISVILTVEAVEHQVKPMFVEKLKNVNIKEGSRLEMKVRATGNPNPDIVWLKNSDIIVPHKYPKIRIEGTKGEAALKIDSTVSQDSAWYTATAINKAGRDTTRCKVNVEVEFAEPEPERKLIIPRGTYRAKEIAAPELEPLHLRYGQEQWEEGDLYDKEKQQKPFFKKKLTSLRLKRFGPAHFECRLTPIGDPTMVVEWLHDGKPLEAANRLRMINEFGYCSLDYGVAYSRDSGIITCRATNKYGTDHTSATLIVKDEKSLVEESQLPEGRKGLQRIEELERMAHEGALTGVTTDQKEKQKPDIVLYPEPVRVLEGETARFRCRVTGYPQPKVNWYLNGQLIRKSKRFRVRYDGIHYLDIVDCKSYDTGEVKVTAENPEGVIEHKVKLEIQQREDFRSVLRRAPEPRPEFHVHEPGKLQFEVQKVDRPVDTTETKEVVKLKRAERITHEKVPEESEELRSKFKRRTEEGYYEAITAVELKSRKKDESYEELLRKTKDELLHWTKELTEEEKKALAEEGKITIPTFKPDKIELSPSMEAPKIFERIQSQTVGQGSDAHFRVRVVGKPDPECEWYKNGVKIERSDRIYWYWPEDNVCELVIRDVTAEDSASIMVKAINIAGETSSHAFLLVQAKQLITFTQELQDVVAKEKDTMATFECETSEPFVKVKWYKDGMEVHEGDKYRMHSDRKVHFLSILTIDTSDAEDYSCVLVEDENVKTTAKLIVEGAVVEFVKELQDIEVPESYSGELECIVSPENIEGKWYHNDVELKSNGKYTITSRRGRQNLTVKDVTKEDQGEYSFVIDGKKTTCKLKMKPRPIAILQGLSDQKVCEGDIVQLEVKVSLESVEGVWMKDGQEVQPSDRVHIVIDKQSHMLLIEDMTKEDAGNYSFTIPALGLSTSGRVSVYSVDVITPLKDVNVIEGTKAVLECKVSVPDVTSVKWYLNDEQIKPDDRVQAIVKGTKQRLVINRTHASDEGPYKLIVGRVETNCNLSVEKIKIIRGLRDLTCTETQNVVFEVELSHSGIDVLWNFKDKEIKPSSKYKIEAHGKIYKLTVLNMMKDDEGKYTFYAGENMTSGKLTVAGGAISKPLTDQTVAESQEAVFECEVANPDSKGEWLRDGKHLPLTNNIRSESDGHKRRLIIAATKLDDIGEYTYKVATSKTSAKLKVEAVKIKKTLKNLTVTETQDAVFTVELTHPNVKGVQWIKNGVVLESNEKYAISVKGTIYSLRIKNCAIVDESVYGFRLGRLGASARLHVETVKIIKKPKDVTALENATVAFEVSVSHDTVPVKWFHKSVEIKPSDKHRLVSERKVHKLMLQNISPSDAGEYTAVVGQLECKAKLFVETLHITKTMKNIEVPETKTASFECEVSHFNVPSMWLKNGVEIEMSEKFKIVVQGKLHQLIIMNTSTEDSAEYTFVCGNDQVSATLTVTPIMITSMLKDINAEEKDTITFEVTVNYEGISYKWLKNGVEIKSTDKCQMRTKKLTHSLNIRNVHFGDAADYTFVAGKATSTATLYVEARHIEFRKHIKDIKVLEKKRAMFECEVSEPDITVQWMKDDQELQITDRIKIQKEKYVHRLLIPSTRMSDAGKYTVVAGGNVSTAKLFVEGRDVRIRSIKKEVQVIEKQRAVVEFEVNEDDVDAHWYKDGIEINFQVQERHKYVVERRIHRMFISETRQSDAGEYTFVAGRNRSSVTLYVNAPEPPQVLQELQPVTVQSGKPARFCAVISGRPQPKISWYKEEQLLSTGFKCKFLHDGQEYTLLLIEAFPEDAAVYTCEAKNDYGVATTSASLSVEVPEVVSPDQEMPVYPPAIITPLQDTVTSEGQPARFQCRVSGTDLKVSWYSKDKKIKPSRFFRMTQFEDTYQLEIAEAYPEDEGTYTFVASNAVGQVSSTANLSLEVQALDRQSSGKDVRESTKSQAVADSSFTKEESKISQKEIKSFQGSSYEYEVQVFESVSQSSIHTAASVQDTQLCHTASLSQIAESTELSKECAKESTGEAPKIFLHLQDVTVKCGDTAQFLCVLKDDSFIDVTWTHEGAKIEESERLKQSQNGNIQFLTICNVQLVDQGLYSCIVHNDCGERTTSAVLSVEGAPESILHERIEQEIEMEMKEFSSSFLSAEEEGLHSAELQLSKINETLELLSESPVYPTKFDSEKEGTGPIFIKEVSNADISMGDVATLSVTVIGIPKPKIQWFFNGVLLTPSADYKFVFDGDDHSLIILFTKLEDEGEYTCMASNDYGKTICSAYLKINSKGEGHKDTETESAVAKSLEKLGGPCPPHFLKELKPIRCAQGLPAIFEYTVVGEPAPTVTWFKENKQLCTSVYYTIIHNPNGSGTFIVNDPQREDSGLYICKAENMLGESTCAAELLVLLEDTDMTDTPCKAKSTPEAPEDFPQTPLKGPAVEALDSEQEIATFVKDTILKAALITEENQQLSYEHIAKANELSSQLPLGAQELQSILEQDKLTPESTREFLCINGSIHFQPLKEPSPNLQLQIVQSQKTFSKEGILMPEEPETQAVLSDTEKIFPSAMSIEQINSLTVEPLKTLLAEPEGNYPQSSIEPPMHSYLTSVAEEVLSPKEKTVSDTNREQRVTLQKQEAQSALILSQSLAEGHVESLQSPDVMISQVNYEPLVPSEHSCTEGGKILIESANPLENAGQDSAVRIEEGKSLRFPLALEEKQVLLKEEHSDNVVMPPDQIIESKREPVAIKKVQEVQGRDLLSKESLLSGIPEEQRLNLKIQICRALQAAVASEQPGLFSEWLRNIEKVEVEAVNITQEPRHIMCMYLVTSAKSVTEEVTIIIEDVDPQMANLKMELRDALCAIIYEEIDILTAEGPRIQQGAKTSLQEEMDSFSGSQKVEPITEPEVESKYLISTEEVSYFNVQSRVKYLDATPVTKGVASAVVSDEKQDESLKPSEEKEESSSESGTEEVATVKIQEAEGGLIKEDGPMIHTPLVDTVSEEGDIVHLTTSITNAKEVNWYFENKLVPSDEKFKCLQDQNTYTLVIDKVNTEDHQGEYVCEALNDSGKTATSAKLTVVKRAAPVIKRKIEPLEVALGHLAKFTCEIQSAPNVRFQWFKAGREIYESDKCSIRSSKYISSLEILRTQVVDCGEYTCKASNEYGSVSCTATLTVTVPGGEKKVRKLLPERKPEPKEEVVLKSVLRKRPEEEEPKVEPKKLEKVKKPAVPEPPPPKPVEEVEVPTVTKRERKIPEPTKVPEIKPAIPLPAPEPKPKPEAEVKTIKPPPVEPEPTPIAAPVTVPVVGKKAEAKAPKEEAAKPKGPIKGVPKKTPSPIEAERRKLRPGSGGEKPPDEAPFTYQLKAVPLKFVKEIKDIILTESEFVGSSAIFECLVSPSTAITTWMKDGSNIRESPKHRFIADGKDRKLHIIDVQLSDAGEYTCVLRLGNKEKTSTAKLVVEELPVRFVKTLEEEVTVVKGQPLYLSCELNKERDVVWRKDGKIVVEKPGRIVPGVIGLMRALTINDADDTDAGTYTVTVENANNLECSSCVKVVEVIRDWLVKPIRDQHVKPKGTAIFACDIAKDTPNIKWFKGYDEIPAEPNDKTEILRDGNHLYLKIKNAMPEDIAEYAVEIEGKRYPAKLTLGEREVELLKPIEDVTIYEKESASFDAEISEADIPGQWKLKGELLRPSPTCEIKAEGGKRFLTLHKVKLDQAGEVLYQALNAITTAILTVKEIELDFAVPLKDVTVPERRQARFECVLTREANVIWSKGPDIIKSSDKFDIIADGKKHILVINDSQFDDEGVYTAEVEGKKTSARLFVTGIRLKFMSPLEDQTVKEGETATFVCELSHEKMHVVWFKNDAKLHTSRTVLISSEGKTHKLEMKEVTLDDISQIKAQVKELSSTAQLKVLEADPYFTVKLHDKTAVEKDEITLKCEVSKDVPVKWFKDGEEIVPSPKYSIKADGLRRILKIKKADLKDKGEYVCDCGTDKTKANVTVEARLIKVEKPLYGVEVFVGETAHFEIELSEPDVHGQWKLKGQPLTASPDCEIIEDGKKHILILHNCQLGMTGEVSFQAANAKSAANLKVKELPLIFITPLSDVKVFEKDEAKFECEVSREPKTFRWLKGTQEITGDDRFELIKDGTKHSMVIKSAAFEDEAKYMFEAEDKHTSGKLIIEGIRLKFLTPLKDVTAKEKESAVFTVELSHDNIRVKWFKNDQRLHTTRSVSMQDEGKTHSITFKDLSIDDTSQIRVEAMGMSSEAKLTVLEGDPYFTGKLQDYTGVEKDEVILQCEISKADAPVKWFKDGKEIKPSKNAVIKADGKKRMLILKKALKSDIGQYTCDCGTDKTSGKLDIEDREIKLVRPLHSVEVMETETARFETEISEDDIHANWKLKGEALLQTPDCEIKEEGKIHSLVLHNCRLDQTGGVDFQAANVKSSAHLRVKPRVIGLLRPLKDVTVTAGETATFDCELSYEDIPVEWYLKGKKLEPSDKVVPRSEGKVHTLTLRDVKLEDAGEVQLTAKDFKTHANLFVKEPPVEFTKPLEDQTVEEGATAVLECEVSRENAKVKWFKNGTEILKSKKYEIVADGRVRKLVIHDCTPEDIKTYTCDAKDFKTSCNLNVVPPHVEFLRPLTDLQVREKEMARFECELSRENAKVKWFKDGAEIKKGKKYDIISKGAVRILVINKCLLDDEAEYSCEVRTARTSGMLTVLEEEAVFTKNLANIEVSETDTIKLVCEVSKPGAEVIWYKGDEEIIETGRYEILTEGRKRILVIQNAHLEDAGNYNCRLPSSRTDGKVKVHELAAEFISKPQNLEILEGEKAEFVCSISKESFPVQWKRDDKTLESGDKYDVIADGKKRVLVVKDATLQDMGTYVVMVGAARAAAHLTVIEKLRIVVPLKDTRVKEQQEVVFNCEVNTEGAKAKWFRNEEAIFDSSKYIILQKDLVYTLRIRDAHLDDQANYNVSLTNHRGENVKSAANLIVEEEDLRIVEPLKDIETMEKKSVTFWCKVNRLNVTLKWTKNGEEVPFDNRVSYRVDKYKHMLTIKDCGFPDEGEYIVTAGQDKSVAELLIIEAPTEFVEHLEDQTVTEFDDAVFSCQLSREKANVKWYRNGREIKEGKKYKFEKDGSIHRLIIKDCRLDDECEYACGVEDRKSRARLFVEEIPVEIIRPPQDILEAPGADVVFLAELNKDKVEVQWLRNNMVVVQGDKHQMMSEGKIHRLQICDIKPRDQGEYRFIAKDKEARAKLELAAAPKIKTADQDLVVDVGKPLTMVVPYDAYPKAEAEWFKENEPLSTKTIDTTAEQTSFRILEAKKGDKGRYKIVLQNKHGKAEGFINLKVIDVPGPVRNLEVTETFDGEVSLAWEEPLTDGGSKIIGYVVERRDIKRKTWVLATDRAESCEFTVTGLQKGGVEYLFRVSARNRVGTGEPVETDNPVEARSKYDVPGPPLNVTITDVNRFGVSLTWEPPEYDGGAEITNYVIELRDKTSIRWDTAMTVRAEDLSATVTDVVEGQEYSFRVRAQNRIGVGKPSAATPFVKVADPIERPSPPVNLTSSDQTQSSVQLKWEPPLKDGGSPILGYIIERCEEGKDNWIRCNMKLVPELTYKVTGLEKGNKYLYRVSAENKAGVSDPSEILGPLTADDAFVEPTMDLSAFKDGLEVIVPNPITILVPSTGYPRPTATWCFGDKVLETGDRVKMKTLSAYAELVISPSERSDKGIYTLKLENRVKTISGEIDVNVIARPSAPKELKFGDITKDSVHLTWEPPDDDGGSPLTGYVVEKREVSRKTWTKVMDFVTDLEFTVPDLVQGKEYLFKVCARNKCGPGEPAYVDEPVNMSTPATVPDPPENVKWRDRTANSIFLTWDPPKNDGGSRIKGYIVERCPRGSDKWVACGEPVAETKMEVTGLEEGKWYAYRVKALNRQGASKPSRPTEEIQAVDTQEAPEIFLDVKLLAGLTVKAGTKIELPATVTGKPEPKITWTKADMILKQDKRITIENVPKKSTVTIVDSKRSDTGTYIIEAVNVCGRATAVVEVNVLDKPGPPAAFDITDVTNESCLLTWNPPRDDGGSKITNYVVERRATDSEVWHKLSSTVKDTNFKATKLIPNKEYIFRVAAENMYGVGEPVQASPITAKYQFDPPGPPTRLEPSDITKDAVTLTWCEPDDDGGSPITGYWVERLDPDTDKWVRCNKMPVKDTTYRVKGLTNKKKYRFRVLAENLAGPGKPSKSTEPILIKDPIDPPWPPGKPTVKDVGKTSVRLNWTKPEHDGGAKIESYVIEMLKTGTDEWVRVAEGVPTTQHLLPGLMEGQEYSFRVRAVNKAGESEPSEPSDPVLCREKLYPPSPPRWLEVINITKNTADLKWTVPEKDGGSPITNYIVEKRDVRRKGWQTVDTTVKDTKCTVTPLTEGSLYVFRVAAENAIGQSDYTEIEDSVLAKDTFTTPGPPYALAVVDVTKRHVDLKWEPPKNDGGRPIQRYVIEKKERLGTRWVKAGKTAGPDCNFRVTDVIEGTEVQFQVRAENEAGVGHPSEPTEILSIEDPTSPPSPPLDLHVTDAGRKHIAIAWKPPEKNGGSPIIGYHVEMCPVGTEKWMRVNSRPIKDLKFKVEEGVVPDKEYVLRVRAVNAIGVSEPSEISENVVAKDPDCKPTIDLETHDIIVIEGEKLSIPVPFRAVPVPTVSWHKDGKEVKASDRLTMKNDHISAHLEVPKSVRADAGIYTITLENKLGSATASINVKVIGLPGPCKDIKASDITKSSCKLTWEPPEFDGGTPILHYVLERREAGRRTYIPVMSGENKLSWTVKDLIPNGEYFFRVKAVNKVGGGEYIELKNPVIAQDPKQPPDPPVDVEVHNPTAEAMTITWKPPLYDGGSKIMGYIIEKIAKGEERWKRCNEHLVPILTYTAKGLEEGKEYQFRVRAENAAGISEPSRATPPTKAVDPIDAPKVILRTSLEVKRGDEIALDASISGSPYPTITWIKDENVIVPEEIKKRAAPLVRRRKGEVQEEEPFVLPLTQRLSIDNSKKGESQLRVRDSLRPDHGLYMIKVENDHGIAKAPCTVSVLDTPGPPINFVFEDIRKTSVLCKWEPPLDDGGSEIINYTLEKKDKTKPDSEWIVVTSTLRHCKYSVTKLIEGKEYLFRVRAENRFGPGPPCVSKPLVAKDPFGPPDAPDKPIVEDVTSNSMLVKWNEPKDNGSPILGYWLEKREVNSTHWSRVNKSLLNALKANVDGLLEGLTYVFRVCAENAAGPGKFSPPSDPKTAHDPISPPGPPIPRVTDTSSTTIELEWEPPAFNGGGEIVGYFVDKQLVGTNEWSRCTEKMIKVRQYTVKEIREGADYKLRVSAVNAAGEGPPGETQPVTVAEPQEPPAVELDVSVKGGIQIMAGKTLRIPAVVTGRPVPTKVWTKEEGELDKDRVVIDNVGTKSELIIKDALRKDHGRYVITATNSCGSKFAAARVEVFDVPGPVLDLKPVVTNRKMCLLNWSDPEDDGGSEITGFIIERKDAKMHTWRQPIETERSKCDITGLLEGQEYKFRVIAKNKFGCGPPVEIGPILAVDPLGPPTSPERLTYTERTKSTITLDWKEPRSNGGSPIQGYIIEKRRHDKPDFERVNKRLCPTTSFLVENLDEHQMYEFRVKAVNEIGESEPSLPLNVVIQDDEVPPTIKLRLSVRGDTIKVKAGEPVHIPADVTGLPMPKIEWSKNETVIEKPTDALQITKEEVSRSEAKTELSIPKAVREDKGTYTVTASNRLGSVFRNVHVEVYDRPSPPRNLAVTDIKAESCYLTWDAPLDNGGSEITHYVIDKRDASRKKAEWEEVTNTAVEKRYGIWKLIPNGQYEFRVRAVNKYGISDECKSDKVVIQDPYRLPGPPGKPKVLARTKGSMLVSWTPPLDNGGSPITGYWLEKREEGSPYWSRVSRAPITKVGLKGVEFNVPRLLEGVKYQFRAMAINAAGIGPPSEPSDPEVAGDPIFPPGPPSCPEVKDKTKSSISLGWKPPAKDGGSPIKGYIVEMQEEGTTDWKRVNEPDKLITTCECVVPNLKELRKYRFRVKAVNEAGESEPSDTTGEIPATDIQEEPEVFIDIGAQDCLVCKAGSQIRIPAVIKGRPTPKSSWEFDGKAKKAMKDGVHDIPEDAQLETAENSSVIIIPECKRSHTGKYSITAKNKAGQKTANCRVKVMDVPGPPKDLKVSDITRGSCRLSWKMPDDDGGDRIKGYVIEKRTIDGKAWTKVNPDCGSTTFVVPDLLSEQQYFFRVRAENRFGIGPPVETIQRTTARDPIYPPDPPIKLKIGLITKNTVHLSWKPPKNDGGSPVTHYIVECLAWDPTGTKKEAWRQCNKRDVEELQFTVEDLVEGGEYEFRVKAVNAAGVSKPSATVGPVTVKDQTCPPSIDLKEFMEVEEGTNVNIVAKIKGVPFPTLTWFKAPPKKPDNKEPVLYDTHVNKLVVDDTCTLVIPQSRRSDTGLYTITAVNNLGTASKEMRLNVLGRPGPPVGPIKFESVSADQMTLSWFPPKDDGGSKITNYVIEKREANRKTWVHVSSEPKECTYTIPKLLEGHEYVFRIMAQNKYGIGEPLDSEPETARNLFSVPGAPDKPTVSSVTRNSMTVNWEEPEYDGGSPVTGYWLEMKDTTSKRWKRVNRDPIKAMTLGVSYKVTGLIEGSDYQFRVYAINAAGVGPASLPSDPATARDPIAPPGPPFPKVTDWTKSSADLEWSPPLKDGGSKVTGYIVEYKEEGKEEWEKGKDKEVRGTKLVVTGLKEGAFYKFRVRAVNIAGIGEPGEVTDVIEMKDRLVSPDLQLDASVRDRIVVHAGGVIRIIAYVSGKPPPTVTWNMNERTLPQEATIETTAISSSMVIKNCQRSHQGVYSLLAKNEAGERKKTIIVDVLDVPGPVGTPFLAHNLTNESCKLTWFSPEDDGGSPITNYVIEKRESDRRAWTPVTYTVTRQNATVQGLIQGKAYFFRIAAENSIGMGPFVETSEALVIREPITVPERPEDLEVKEVTKNTVTLTWNPPKYDGGSEIINYVLESRLIGTEKFHKVTNDNLLSRKYTVKGLKEGDTYEYRVSAVNIVGQGKPSFCTKPITCKDELAPPTLHLDFRDKLTIRVGEAFALTGRYSGKPKPKVSWFKDEADVLEDDRTHIKTTPATLALEKIKAKRSDSGKYCVVVENSTGSRKGFCQVNVVDRPGPPVGPVSFDEVTKDYMVISWKPPLDDGGSKITNYIIEKKEVGKDVWMPVTSASAKTTCKVSKLLEGKDYIFRIHAENLYGISDPLVSDSMKAKDRFRVPDAPDQPIVTEVTKDSALVTWNKPHDGGKPITNYILEKRETMSKRWARVTKDPIHPYTKFRVPDLLEGCQYEFRVSAENEIGIGDPSPPSKPVFAKDPIAKPSPPVNPEAIDTTCNSVDLTWQPPRHDGGSKILGYIVEYQKVGDEEWRRANHTPESCPETKYKVTGLRDGQTYKFRVLAVNAAGESDPAHVPEPVLVKDRLEPPELILDANMAREQHIKVGDTLRLSAIIKGVPFPKVTWKKEDRDAPTKARIDVTPVGSKLEIRNAAHEDGGIYSLTVENPAGSKTVSVKVLVLDKPGPPRDLEVSEIRKDSCYLTWKEPLDDGGSVITNYVVERRDVASAQWSPLSATSKKKSHFAKHLNEGNQYLFRVAAENQYGRGPFVETPKPIKALDPLHPPGPPKDLHHVDVDKTEVSLVWNKPDRDGGSPITGYLVEYQEEGTQDWIKFKTVTNLECVVTGLQQGKTYRFRVKAENIVGLGLPDTTIPIECQEKLVPPSVELDVKLIEGLVVKAGTTVRFPAIIRGVPVPTAKWTTDGSEIKTDEHYTVETDNFSSVLTIKNCLRRDTGEYQITVSNAAGSKTVAVHLTVLDVPGPPTGPINILDVTPEHMTISWQPPKDDGGSPVINYIVEKQDTRKDTWGVVSSGSSKTKLKIPHLQKGCEYVFRVRAENKIGVGPPLDSTPTVAKHKFSPPSPPGKPVVTDITENAATVSWTLPKSDGGSPITGYYMERREVTGKWVRVNKTPIADLKFRVTGLYEGNTYEFRVFAENLAGLSKPSPSSDPIKACRPIKPPGPPINPKLKDKSRETADLVWTKPLSDGGSPILGYVVECQKPGTAQWNRINKDELIRQCAFRVPGLIEGNEYRFRIKAANIVGEGEPRELAESVIAKDILHPPEVELDVTCRDVITVRVGQTIRILARVKGRPEPDITWTKEGKVLVREKRVDLIQDLPRVELQIKEAVRADHGKYIISAKNSSGHAQGSAIVNVLDRPGPCQNLKVTNVTKENCTISWENPLDNGGSEITNFIVEYRKPNQKGWSIVASDVTKRLIKANLLANNEYYFRVCAENKVGVGPTIETKTPILAINPIDRPGEPENLHIADKGKTFVYLKWRRPDYDGGSPNLSYHVERRLKGSDDWERVHKGSIKETHYMVDRCVENQIYEFRVQTKNEGGESDWVKTEEVVVKEDLQKPVLDLKLSGVLTVKAGDTIRLEAGVRGKPFPEVAWTKDKDATDLTRSPRVKIDTRADSSKFSLTKAKRSDGGKYVVTATNTAGSFVAYATVNVLDKPGPVRNLKIVDVSSDRCTVCWDPPEDDGGCEIQNYILEKCETKRMVWSTYSATVLTPGTTVTRLIEGNEYIFRVRAENKIGTGPPTESKPVIAKTKYDKPGRPDPPEVTKVSKEEMTVVWNPPEYDGGKSITGYFLEKKEKHSTRWVPVNKSAIPERRMKVQNLLPDHEYQFRVKAENEIGIGEPSLPSRPVVAKDPIEPPGPPTNFRVVDTTKHSITLGWGKPVYDGGAPIIGYVVEMRPKIADASPDEGWKRCNAAAQLVRKEFTVTSLDENQEYEFRVCAQNQVGIGRPAELKEAIKPKEILEPPEIDLDASMRKLVIVRAGCPIRLFAIVRGRPAPKVTWRKVGIDNVVRKGQVDLVDTMAFLVIPNSTRDDSGKYSLTLVNPAGEKAVFVNVRVLDTPGPVSDLKVSDVTKTSCHVSWAPPENDGGSQVTHYIVEKREADRKTWSTVTPEVKKTSFHVTNLVPGNEYYFRVTAVNEYGPGVPTDVPKPVLASDPLSEPDPPRKLEVTEMTKNSATLAWLPPLRDGGAKIDGYITSYREEEQPADRWTEYSVVKDLSLVVTGLKEGKKYKFRVAARNAVGVSLPREAEGVYEAKEQLLPPKILMPEQITIKAGKKLRIEAHVYGKPHPTCKWKKGEDEVVTSSHLAVHKADSSSILIIKDVTRKDSGYYSLTAENSSGTDTQKIKVVVMDAPGPPQPPFDISDIDADACSLSWHIPLEDGGSNITNYIVEKCDVSRGDWVTALASVTKTSCRVGKLIPGQEYIFRVRAENRFGISEPLTSPKMVAQFPFGVPSEPKNARVTKVNKDCIFVAWDRPDSDGGSPIIGYLIERKERNSLLWVKANDTLVRSTEYPCAGLVEGLEYSFRIYALNKAGSSPPSKPTEYVTARMPVDPPGKPEVIDVTKSTVSLIWARPKHDGGSKIIGYFVEACKLPGDKWVRCNTAPHQIPQEEYTATGLEEKAQYQFRAIARTAVNISPPSEPSDPVTILAENVPPRIDLSVAMKSLLTVKAGTNVCLDATVFGKPMPTVSWKKDGTLLKPAEGIKMAMQRNLCTLELFSVNRKDSGDYTITAENSSGSKSATIKLKVLDKPGPPASVKINKMYSDRAMLSWEPPLEDGGSEITNYIVDKRETSRPNWAQVSATVPITSCSVEKLIEGHEYQFRICAENKYGVGDPVFTEPAIAKNPYDPPGRCDPPVISNITKDHMTVSWKPPADDGGSPITGYLLEKRETQAVNWTKVNRKPIIERTLKATGLQEGTEYEFRVTAINKAGPGKPSDASKAAYARDPQYPPGPPAFPKVYDTTRSSVSLSWGKPAYDGGSPIIGYLVEVKRADSDNWVRCNLPQNLQKTRFEVTGLMEDTQYQFRVYAVNKIGYSDPSDVPDKHYPKDILIPPEGELDADLRKTLILRAGVTMRLYVPVKGRPPPKITWSKPNVNLRDRIGLDIKSTDFDTFLRCENVNKYDAGKYILTLENSCGKKEYTIVVKVLDTPGPPVNVTVKEISKDSAYVTWEPPIIDGGSPIINYVVQKRDAERKSWSTVTTECSKTSFRVANLEEGKSYFFRVFAENEYGIGDPGETRDAVKASQTPGPVVDLKVRSVSKSSCSIGWKKPHSDGGSRIIGYVVDFLTEENKWQRVMKSLSLQYSAKDLTEGKEYTFRVSAENENGEGTPSEITVVARDDVVAPDLDLKGLPDLCYLAKENSNFRLKIPIKGKPAPSVSWKKGEDPLATDTRVSVESSAVNTTLIVYDCQKSDAGKYTITLKNVAGTKEGTISIKVVGKPGIPTGPIKFDEVTAEAMTLKWAPPKDDGGSEITNYILEKRDSVNNKWVTCASAVQKTTFRVTRLHEGMEYTFRVSAENKYGVGEGLKSEPIVARHPFDVPDAPPPPNIVDVRHDSVSLTWTDPKKTGGSPITGYHLEFKERNSLLWKRANKTPIRMRDFKVTGLTEGLEYEFRVMAINLAGVGKPSLPSEPVVALDPIDPPGKPEVINITRNSVTLIWTEPKYDGGHKLTGYIVEKRDLPSKSWMKANHVNVPECAFTVTDLVEGGKYEFRIRAKNTAGAISAPSESTETIICKDEYEAPTIVLDPTIKDGLTIKAGDTIVLNAISILGKPLPKSSWSKAGKDIRPSDITQITSTPTSSMLTIKYATRKDAGEYTITATNPFGTKVEHVKVTVLDVPGPPGPVEISNVSAEKATLTWTPPLEDGGSPIKSYILEKRETSRLLWTVVSEDIQSCRHVATKLIQGNEYIFRVSAVNHYGKGEPVQSEPVKMVDRFGPPGPPEKPEVSNVTKNTATVSWKRPVDDGGSEITGYHVERREKKSLRWVRAIKTPVSDLRCKVTGLQEGSTYEFRVSAENRAGIGPPSEASDSVLMKDAAYPPGPPSNPHVTDTTKKSASLAWGKPHYDGGLEITGYVVEHQKVGDEAWIKDTTGTALRITQFVVPDLQTKEKYNFRISAINDAGVGEPAVIPDVEIVEREMAPDFELDAELRRTLVVRAGLSIRIFVPIKGRPAPEVTWTKDNINLKNRANIENTESFTLLIIPECNRYDTGKFVMTIENPAGKKSGFVNVRVLDTPGPVLNLRPTDITKDSVTLHWDLPLIDGGSRITNYIVEKREATRKSYSTATTKCHKCTYKVTGLSEGCEYFFRVMAENEYGIGEPTETTEPVKASEAPSPPDSLNIMDITKSTVSLAWPKPKHDGGSKITGYVIEAQRKGSDQWTHITTVKGLECVVRNLTEGEEYTFQVMAVNSAGRSAPRESRPVIVKEQTMLPELDLRGIYQKLVIAKAGDNIKVEIPVLGRPKPTVTWKKGDQILKQTQRVNFETTATSTILNINECVRSDSGPYPLTARNIVGEVGDVITIQVHDIPGPPTGPIKFDEVSSDFVTFSWDPPENDGGVPISNYVVEMRQTDSTTWVELATTVIRTTYKATRLTTGLEYQFRVKAQNRYGVGPGITSACIVANYPFKVPGPPGTPQVTAVTKDSMTISWHEPLSDGGSPILGYHVERKERNGILWQTVSKALVPGNIFKSSGLTDGIAYEFRVIAENMAGKSKPSKPSEPMLALDPIDPPGKPVPLNITRHTVTLKWAKPEYTGGFKITSYIVEKRDLPNGRWLKANFSNILENEFTVSGLTEDAAYEFRVIAKNAAGAISPPSEPSDAITCRDDVEAPKIKVDVKFKDTVILKAGEAFRLEADVSGRPPPTMEWSKDGKELEGTAKLEIKIADFSTNLVNKDSTRRDSGAYTLTATNPGGFAKHIFNVKVLDRPGPPEGPLAVTEVTSEKCVLSWFPPLDDGGAKIDHYIVQKRETSRLAWTNVASEVQVTKLKVTKLLKGNEYIFRVMAVNKYGVGEPLESEPVLAVNPYGPPDPPKNPEVTTITKDSMVVCWGHPDSDGGSEIINYIVERRDKAGQRWIKCNKKTLTDLRYKVSGLTEGHEYEFRIMAENAAGISAPSPTSPFYKACDTVFKPGPPGNPRVLDTSRSSISIAWNKPIYDGGSEITGYMVEIALPEEDEWQIVTPPAGLKATSYTITGLTENQEYKIRIYAMNSEGLGEPALVPGTPKAEDRMLPPEIELDADLRKVVTIRACCTLRLFVPIKGRPAPEVKWARDHGESLDKASIESTSSYTLLIVGNVNRFDSGKYILTVENSSGSKSAFVNVRVLDTPGPPQDLKVKEVTKTSVTLTWDPPLLDGGSKIKNYIVEKRESTRKAYSTVATNCHKTSWKVDQLQEGCSYYFRVLAENEYGIGLPAETAESVKASERPLPPGKITLMDVTRNSVSLSWEKPEHDGGSRILGYIVEMQTKGSDKWATCATVKVTEATITGLIQGEEYSFRVSAQNEKGISDPRQLSVPVIAKDLVIPPAFKLLFNTFTVLAGEDLKVDVPFIGRPTPAVTWHKDNVPLKQTTRVNAESTENNSLLTIKDACREDVGHYVVKLTNSAGEAIETLNVIVLDKPGPPTGPVKMDEVTADSITLSWGPPKYDGGSSINNYIVEKRDTSTTTWQIVSATVARTTIKACRLKTGCEYQFRIAAENRYGKSTYLNSEPTVAQYPFKVPGPPGTPVVTLSSRDSMEVQWNEPISDGGSRVIGYHLERKERNSILWVKLNKTPIPQTKFKTTGLEEGVEYEFRVSAENIVGIGKPSKVSECYVARDPCDPPGRPEAIIVTRNSVTLQWKKPTYDGGSKITGYIVEKKELPEGRWMKASFTNIIDTHFEVTGLVEDHRYEFRVIARNAAGVFSEPSESTGAITARDEVDPPRISMDPKYKDTIVVHAGESFKVDADIYGKPIPTIQWIKGDQELSNTARLEIKSTDFATSLSVKDAVRVDSGNYILKAKNVAGERSVTVNVKVLDRPGPPEGPVVISGVTAEKCTLAWKPPLQDGGSDIINYIVERRETSRLVWTVVDANVQTLSCKVTKLLEGNEYTFRIMAVNKYGVGEPLESEPVVAKNPFVVPDAPKAPEVTTVTKDSMIVVWERPASDGGSEILGYVLEKRDKEGIRWTRCHKRLIGELRLRVTGLIENHDYEFRVSAENAAGLSEPSPPSAYQKACDPIYKPGPPNNPKVIDITRSSVFLSWSKPIYDGGCEIQGYIVEKCDVSVGEWTMCTPPTGINKTNIEVEKLLEKHEYNFRICAINKAGVGEHADVPGPIIVEEKLEAPDIDLDLELRKIINIRAGGSLRLFVPIKGRPTPEVKWGKVDGEIRDAAIIDVTSSFTSLVLDNVNRYDSGKYTLTLENSSGTKSAFVTVRVLDTPSPPVNLKVTEITKDSVSITWEPPLLDGGSKIKNYIVEKREATRKSYAAVVTNCHKNSWKIDQLQEGCSYYFRVTAENEYGIGLPAQTADPIKVAEVPQPPGKITVDDVTRNSVSLSWTKPEHDGGSKIIQYIVEMQAKHSEKWSECARVKSLQAVITNLTQGEEYLFRVVAVNEKGRSDPRSLAVPIVAKDLVIEPDVKPAFSSYSVQVGQDLKIEVPISGRPKPTITWTKDGLPLKQTTRINVTDSLDLTTLSIKETHKDDGGQYGITVANVVGQKTASIEIVTLDKPDPPKGPVKFDDVSAESITLSWNPPLYTGGCQITNYIVQKRDTTTTVWDVVSATVARTTLKVTKLKTGTEYQFRIFAENRYGQSFALESDPIVAQYPYKEPGPPGTPFATAISKDSMVIQWHEPVNNGGSPVIGYHLERKERNSILWTKVNKTIIHDTQFKAQNLEEGIEYEFRVYAENIVGVGKASKNSECYVARDPCDPPGTPEPIMVKRNEITLQWTKPVYDGGSMITGYIVEKRDLPDGRWMKASFTNVIETQFTVSGLTEDQRYEFRVIAKNAAGAISKPSDSTGPITAKDEVELPRISMDPKFRDTIVVNAGETFRLEADVHGKPLPTIEWLRGDKEIEESARCEIKNTDFKALLIVKDAIRIDGGQYILRASNVAGSKSFPVNVKVLDRPGPPEGPVQVTGVTSEKCSLTWSPPLQDGGSDISHYVVEKRETSRLAWTVVASEVVTNSLKVTKLLEGNEYVFRIMAVNKYGVGEPLESAPVLMKNPFVLPGPPKSLEVTNIAKDSMTVCWNRPDSDGGSEIIGYIVEKRDRSGIRWIKCNKRRITDLRLRVTGLTEDHEYEFRVSAENAAGVGEPSPATVYYKACDPVFKPGPPTNAHIVDTTKNSITLAWGKPIYDGGSEILGYVVEICKADEEEWQIVTPQTGLRVTRFEISKLTEHQEYKIRVCALNKVGLGEATSVPGTVKPEDKLEAPELDLDSELRKGIVVRAGGSARIHIPFKGRPTPEITWSREEGEFTDKVQIEKGVNYTQLSIDNCDRNDAGKYILKLENSSGSKSAFVTVKVLDTPGPPQNLAVKEVRKDSAFLVWEPPIIDGGAKVKNYVIDKRESTRKAYANVSSKCSKTSFKVENLTEGAIYYFRVMAENEFGVGVPVETVDAVKAAEPPSPPGKVTLTDVSQTSASLMWEKPEHDGGSRVLGYVVEMQPKGTEKWSIVAESKVCNAVVTGLSSGQEYQFRVKAYNEKGKSDPRVLGVPVIAKDLTIQPSLKLPFNTYSIQAGEDLKIEIPVIGRPRPNISWVKDGEPLKQTTRVNVEETATSTVLHIKEGNKDDFGKYTVTATNSAGTATENLSVIVLEKPGPPVGPVRFDEVSADFVVISWEPPAYTGGCQISNYIVEKRDTTTTTWHMVSATVARTTIKITKLKTGTEYQFRIFAENRYGKSAPLDSKAVIVQYPFKEPGPPGTPFVTSISKDQMLVQWHEPVNDGGTKIIGYHLEQKEKNSILWVKLNKTPIQDTKFKTTGLDEGLEYEFKVSAENIVGIGKPSKVSECFVARDPCDPPGRPEAIVITRNNVTLKWKKPAYDGGSKITGYIVEKKDLPDGRWMKASFTNVLETEFTVSGLVEDQRYEFRVIARNAAGNFSEPSDSSGAITARDEIDAPNASLDPKYKDVIVVHAGETFVLEADIRGKPIPDVVWSKDGKELEETAARMEIKSTIQKTTLVVKDCIRTDGGQYILKLSNVGGTKSIPITVKVLDRPGPPEGPLKVTGVTAEKCYLAWNPPLQDGGANISHYIIEKRETSRLSWTQVSTEVQALNYKVTKLLPGNEYIFRVMAVNKYGIGEPLESGPVTACNPYKPPGPPSTPEVSAITKDSMVVTWARPVDDGGTEIEGYILEKRDKEGVRWTKCNKKTLTDLRLRVTGLTEGHSYEFRVAAENAAGVGEPSEPSVFYRACDALYPPGPPSNPKVTDTSRSSVSLAWSKPIYDGGAPVKGYVVEVKEAAADEWTTCTPPTGLQGKQFTVTKLKENTEYNFRICAINSEGVGEPATLPGSVVAQERIEPPEIELDADLRKVVVLRASATLRLFVTIKGRPEPEVKWEKAEGILTDRAQIEVTSSFTMLVIDNVTRFDSGRYNLTLENNSGSKTAFVNVRVLDSPSAPVNLTIREVKKDSVTLSWEPPLIDGGAKITNYIVEKRETTRKAYATITNNCTKTTFRIENLQEGCSYYFRVLASNEYGIGLPAETTEPVKVSEPPLPPGRVTLVDVTRNTATIKWEKPESDGGSKITGYVVEMQTKGSEKWSTCTQVKTLEATISGLTAGEEYVFRVAAVNEKGRSDPRQLGVPVIARDIEIKPSVELPFHTFNVKAREQLKIDVPFKGRPQATVNWRKDGQTLKETTRVNVSSSKTVTSLSIKEASKEDVGTYELCVSNSAGSITVPITIIVLDRPGPPGPIRIDEVSCDSITISWNPPEYDGGCQISNYIVEKKETTSTTWHIVSQAVARTSIKIVRLTTGSEYQFRVCAENRYGKSSYSESSAVVAEYPFSPPGPPGTPKVVHATKSTMLVTWQVPVNDGGSRVIGYHLEYKERSSILWSKANKILIADTQMKVSGLDEGLMYEYRVYAENIAGIGKCSKSCEPVPARDPCDPPGQPEVTNITRKSVSLKWSKPHYDGGAKITGYIVERRELPDGRWLKCNYTNIQETYFEVTELTEDQRYEFRVFARNAADSVSEPSESTGPIIVKDDVEPPRVMMDVKFRDVIVVKAGEVLKINADIAGRPLPVISWAKDGIEIEERARTEIISTDNHTLLTVKDCIRRDTGQYVLTLKNVAGTRSVAVNCKVLDKPGPPAGPLEINGLTAEKCSLSWGRPQEDGGADIDYYIVEKRETSHLAWTICEGELQMTSCKVTKLLKGNEYIFRVTGVNKYGVGEPLESVAIKALDPFTVPSPPTSLEITSVTKESMTLCWSRPESDGGSEISGYIIERREKNSLRWVRVNKKPVYDLRVKSTGLREGCEYEYRVYAENAAGLSLPSETSPLIRAEDPVFLPSPPSKPKIVDSGKTTITIAWVKPLFDGGAPITGYTVEYKKSDDTDWKTSIQSLRGTEYTISGLTTGAEYVFRVKSVNKVGASDPSDSSDPQIAKEREEEPLFDIDSEMRKTLIVKAGASFTMTVPFRGRPVPNVLWSKPDTDLRTRAYVDTTDSRTSLTIENANRNDSGKYTLTIQNVLSAASLTLVVKVLDTPGPPTNITVQDVTKESAVLSWDVPENDGGAPVKNYHIEKREASKKAWVSVTNNCNRLSYKVTNLQEGAIYYFRVSGENEFGVGIPAETKEGVKITEKPSPPEKLGVTSISKDSVSLTWLKPEHDGGSRIVHYVVEALEKGQKNWVKCAVAKSTHHVVSGLRENSEYFFRVFAENQAGLSDPRELLLPVLIKEQLEPPEIDMKNFPSHTVYVRAGSNLKVDIPISGKPLPKVTLSRDGVPLKATMRFNTEITAENLTINLKESVTADAGRYEITAANSSGTTKAFINIVVLDRPGPPTGPVVISDITEESVTLKWEPPKYDGGSQVTNYILLKRETSTAVWTEVSATVARTMMKVMKLTTGEEYQFRIKAENRFGISDHIDSACVTVKLPYTTPGPPSTPWVTNVTRESITVGWHEPVSNGGSAVVGYHLEMKDRNSILWQKANKLVIRTTHFKVTTISAGLIYEFRVYAENAAGVGKPSHPSEPVLAIDACEPPRNVRITDISKNSVSLSWQQPAFDGGSKITGYIVERRDLPDGRWTKASFTNVTETQFIISGLTQNSQYEFRVFARNAVGSISNPSEVVGPITCIDSYGGPVIDLPLEYTEVVKYRAGTSVKLRAGISGKPAPTIEWYKDDKELQTNALVCVENTTDLASILIKDADRLNSGCYELKLRNAMGSASATIRVQILDKPGPPGGPIEFKTVTAEKITLLWRPPADDGGAKITHYIVEKRETSRVVWSMVSEHLEECIITTTKIIKGNEYIFRVRAVNKYGIGEPLESDSVVAKNAFVTPGPPGIPEVTKITKNSMTVVWSRPIADGGSDISGYFLEKRDKKSLGWFKVLKETIRDTRQKVTGLTENSDYQYRVCAVNAAGQGPFSEPSEFYKAADPIDPPGPPAKIRIADSTKSSITLGWSKPVYDGGSAVTGYVVEIRQGEEEEWTTVSTKGEVRTTEYVVSNLKPGVNYYFRVSAVNCAGQGEPIEMNEPVQAKDILEAPEIDLDVALRTSVIAKAGEDVQVLIPFKGRPPPTVTWRKDEKNLGSDARYSIENTDSSSLLTIPQVTRNDTGKYILTIENGVGEPKSSTVSVKVLDTPAACQKLQVKHVSRGTVTLLWDPPLIDGGSPIINYVIEKRDATKRTWSVVSHKCSSTSFKLIDLSEKTPFFFRVLAENEIGIGEPCETTEPVKAAEVPAPIRDLSMKDSTKTSVILSWTKPDFDGGSVITEYVVERKGKGEQTWSHAGISKTCEIEVSQLKEQSVLEFRVFAKNEKGLSDPVTIGPITVKELIITPEVDLSDIPGAQVTVRIGHNVHLELPYKGKPKPSISWLKDGLPLKESEFVRFSKTENKITLSIKNAKKEHGGKYTVILDNAVCRIAVPITVITLGPPSKPKGPIRFDEIKADSVILSWDVPEDNGGGEITCYSIEKRETSQTNWKMVCSSVARTTFKVPNLVKDAEYQFRVRAENRYGVSQPLVSSIIVAKHQFRIPGPPGKPVIYNVTSDGMSLTWDAPVYDGGSEVTGFHVEKKERNSILWQKVNTSPISGREYRATGLVEGLDYQFRVYAENSAGLSSPSDPSKFTLAVSPVDPPGTPDYIDVTRETITLKWNPPLRDGGSKIVGYSIEKRQGNERWVRCNFTDVSECQYTVTGLSPGDRYEFRIIARNAVGTISPPSQSSGIIMTRDENVPPIVEFGPEYFDGLIIKSGESLRIKALVQGRPVPRVTWFKDGVEIEKRMNMEITDVLGSTSLFVRDATRDHRGVYTVEAKNASGSAKAEIKVKVQDTPGKVVGPIRFTNITGEKMTLWWDAPLNDGCAPITHYIIEKRETSRLAWALIEDKCEAQSYTAIKLINGNEYQFRVSAVNKFGVGRPLDSDPVVAQIQYTVPDAPGIPEPSNITGNSITLTWARPESDGGSEIQQYILERREKKSTRWVKVISKRPISETRFKVTGLTEGNEYEFHVMAENAAGVGPASGISRLIKCREPVNPPGPPTVVKVTDTSKTTVSLEWSKPVFDGGMEIIGYIIEMCKADLGDWHKVNAEACVKTRYTVTDLQAGEEYKFRVSAINGAGKGDSCEVTGTIKAVDRLTAPELDIDANFKQTHVVRAGASIRLFIAYQGRPTPTAVWSKPDSNLSLRADIHTTDSFSTLTVENCNRNDAGKYTLTVENNSGSKSITFTVKVLDTPGPPGPITFKDVTRGSATLMWDAPLLDGGARIHHYVVEKREASRRSWQVISEKCTRQIFKVNDLAEGVPYYFRVSAVNEYGVGEPYEMPEPIVATEQPAPPRRLDVVDTSKSSAVLAWLKPDHDGGSRITGYLLEMRQKGSDFWVEAGHTKQLTFTVERLVEKTEYEFRVKAKNDAGYSEPREAFSSVIIKEPQIEPTADLTGITNQLITCKAGSPFTIDVPISGRPAPKVTWKLEEMRLKETDRVSITTTKDRTTLTVKDSMRGDSGRYFLTLENTAGVKTFSVTVVVIGRPGPVTGPIEVSSVSAESCVLSWGEPKDGGGTEITNYIVEKRESGTTAWQLVNSSVKRTQIKVTHLTKYMEYSFRVSSENRFGVSKPLESAPIIAEHPFVPPSAPTRPEVYHVSANAMSIRWEEPYHDGGSKIIGYWVEKKERNTILWVKENKVPCLECNYKVTGLVEGLEYQFRTYALNAAGVSKASEASRPIMAQNPVDAPGRPEVTDVTRSTVSLIWSAPAYDGGSKVVGYIIERKPVSEVGDGRWLKCNYTIVSDNFFTVTALSEGDTYEFRVLAKNAAGVISKGSESTGPVTCRDEYAPPKAELDARLHGDLVTIRAGSDLVLDAAVGGKPEPKIIWTKGDKELDLCEKVSLQYTGKRATAVIKFCDRSDSGKYTLTVKNASGTKAVSVMVKVLDSPGPCGKLTVSRVTQEKCTLAWSLPQEDGGAEITHYIVERRETSRLNWVIVEGECPTLSYVVTRLIKNNEYIFRVRAVNKYGPGVPVESEPIVARNSFTIPSPPGIPEEVGTGKEHIIIQWTKPESDGGNEISNYLVDKREKKSLRWTRVNKDYVVYDTRLKVTSLMEGCDYQFRVTAVNAAGNSEPSEASNFISCREPSYTPGPPSAPRVVDTTKHSISLAWTKPMYDGGTDIVGYVLEMQEKDTDQWYRVHTNATIRNTEFTVPDLKMGQKYSFRVAAVNVKGMSEYSESIAEIEPVERIEIPDLELADDLKKTVTIRAGASLRLMVSVSGRPPPVITWSKQGIDLASRAIIDTTESYSLLIVDKVNRYDAGKYTIEAENQSGKKSATVLVKVYDTPGPCPSVKVKEVSRDSVTITWEIPTIDGGAPVNNYIVEKREAAMRAFKTVTTKCSKTLYRISGLVEGTMYYFRVLPENIYGIGEPCETSDAVLVSEVPLVPAKLEVVDVTKSTVTLAWEKPLYDGGSRLTGYVLEACKAGTERWMKVVTLKPTVLEHTVTSLNEGEQYLFRIRAQNEKGVSEPRETVTAVTVQDLRVLPTIDLSTMPQKTIHVPAGRPVELVIPIAGRPPPAASWFFAGSKLRESERVTVETHTKVAKLTIRETTIRDTGEYTLELKNVTGTTSETIKVIILDKPGPPTGPIKIDEIDATSITISWEPPELDGGAPLSGYVVEQRDAHRPGWLPVSESVTRSTFKFTRLTEGNEYVFRVAATNRFGIGSYLQSEVIECRSSIRIPGPPETLQIFDVSRDGMTLTWYPPEDDGGSQVTGYIVERKEVRADRWVRVNKVPVTMTRYRSTGLTEGLEYEHRVTAINARGSGKPSRPSKPIVAMDPIAPPGKPQNPRVTDTTRTSVSLAWSVPEDEGGSKVTGYLIEMQKVDQHEWTKCNTTPTKIREYTLTHLPQGAEYRFRVLACNAGGPGEPAEVPGTVKVTEMLEYPDYELDERYQEGIFVRQGGVIRLTIPIKGKPFPICKWTKEGQDISKRAMIATSETHTELVIKEADRGDSGTYDLVLENKCGKKAVYIKVRVIGSPNSPEGPLEYDDIQVRSVRVSWRPPADDGGADILGYILERREVPKAAWYTIDSRVRGTSLVVKGLKENVEYHFRVSAENQFGISKPLKSEEPVTPKTPLNPPEPPSNPPEVLDVTKSSVSLSWSRPKDDGGSRVTGYYIERKETSTDKWVRHNKTQITTTMYTVTGLVPDAEYQFRIIAQNDVGLSETSPASEPVVCKDPFDKPSQPGELEILSISKDSVTLQWEKPECDGGKEILGYWVEYRQSGDSAWKKSNKERIKDKQFTIGGLLEATEYEFRVFAENETGLSRPRRTAMSIKTKLTSGEAPGIRKEMKDVTTKLGEAAQLSCQIVGRPLPDIKWYRFGKELIQSRKYKMSSDGRTHTLTVMTEEQEDEGVYTCIATNEVGEVETSSKLLLQATPQFHPGYPLKEKYYGAVGSTLRLHVMYIGRPVPAMTWFHGQKLLQNSENITIENTEHYTHLVMKNVQRKTHAGKYKVQLSNVFGTVDAILDVEIQDKPDKPTGPIVIEALLKNSAVISWKPPADDGGSWITNYVVEKCEAKEGAEWQLVSSAISVTTCRIVNLTENAGYYFRVSAQNTFGISDPLEVSSVVIIKSPFEKPGAPGKPTITAVTKDSCVVAWKPPASDGGAKIRNYYLEKREKKQNKWISVTTEEIRETVFSVKNLIEGLEYEFRVKCENLGGESEWSEISEPITPKSDVPIQAPHFKEELRNLNVRYQSNATLVCKVTGHPKPIVKWYRQGKEIIADGLKYRIQEFKGGYHQLIIASVTDDDATVYQVRATNQGGSVSGTASLEVEVPAKIHLPKTLEGMGAVHALRGEVVSIKIPFSGKPDPVITWQKGQDLIDNNGHYQVIVTRSFTSLVFPNGVERKDAGFYVVCAKNRFGIDQKTVELDVADVPDPPRGVKVSDVSRDSVNLTWTEPASDGGSKITNYIVEKCATTAERWLRVGQARETRYTVINLFGKTSYQFRVIAENKFGLSKPSEPSEPTITKEDKTRAMNYDEEVDETREVSMTKASHSSTKELYEKYMIAEDLGRGEFGIVHRCVETSSKKTYMAKFVKVKGTDQVLVKKEISILNIARHRNILHLHESFESMEELVMIFEFISGLDIFERINTSAFELNEREIVSYVHQVCEALQFLHSHNIGHFDIRPENIIYQTRRSSTIKIIEFGQARQLKPGDNFRLLFTAPEYYAPEVHQHDVVSTATDMWSLGTLVYVLLSGINPFLAETNQQIIENIMNAEYTFDEEAFKEISIEAMDFVDRLLVKERKSRMTASEALQHPWLKQKIERVSTKVIRTLKHRRYYHTLIKKDLNMVVSAARISCGGAIRSQKGVSVAKVKVASIEIGPVSGQIMHAVGEEGGHVKYVCKIENYDQSTQVTWYFGVRQLENSEKYEITYEDGVAILYVKDITKLDDGTYRCKVVNDYGEDSSYAELFVKGVREVYDYYCRRTMKKIKRRTDTMRLLERPPEFTLPLYNKTAYVGENVRFGVTITVHPEPHVTWYKSGQKIKPGDNDKKYTFESDKGLYQLTINSVTTDDDAEYTVVARNKYGEDSCKAKLTVTLHPPPTDSTLRPMFKRLLANAECQEGQSVCFEIRVSGIPPPTLKWEKDGQPLSLGPNIEIIHEGLDYYALHIRDTLPEDTGYYRVTATNTAGSTSCQAHLQVERLRYKKQEFKSKEEHERHVQKQIDKTLRMAEILSGTESVPLTQVAKEALREAAVLYKPAVSTKTVKGEFRLEIEEKKEERKLRMPYDVPEPRKYKQTTIEEDQRIKQFVPMSDMKWYKKIRDQYEMPGKLDRVVQKRPKRIRLSRWEQFYVMPLPRITDQYRPKWRIPKLSQDDLEIVRPARRRTPSPDYDFYYRPRRRSLGDISDEELLLPIDDYLAMKRTEEERLRLEEELELGFSASPPSRSPPHFELSSLRYSSPQAHVKVEETRKDFRYSTYHIPTKAEASTSYAELRERHAQAAYRQPKQRQRIMAEREDEELLRPVTTTQHLSEYKSELDFMSKEEKSRKKSRRQREVTEITEIEEEYEISKHAQRESSSSASRLLRRRRSLSPTYIELMRPVSELIRSRPQPAEEYEDDTERRSPTPERTRPRSPSPVSSERSLSRFERSARFDIFSRYESMKAALKTQKTSERKYEVLSQQPFTLDHAPRITLRMRSHRVPCGQNTRFILNVQSKPTAEVKWYHNGVELQESSKIHYTNTSGVLTLEILDCHTDDSGTYRAVCTNYKGEASDYATLDVTGGDYTTYASQRRDEEVPRSVFPELTRTEAYAVSSFKKTSEMEASSSVREVKSQMTETRESLSSYEHSASAEMKSAALEEKSLEEKSTTRKIKTTLAARILTKPRSMTVYEGESARFSCDTDGEPVPTVTWLRKGQVLSTSARHQVTTTKYKSTFEISSVQASDEGNYSVVVENSEGKQEAEFTLTIQKARVTEKAVTSPPRVKSPEPRVKSPEAVKSPKRVKSPEPSHPKAVSPTETKPTPTEKVQHLPVSAPPKITQFLKAEASKEIAKLTCVVESSVLRAKEVTWYKDGKKLKENGHFQFHYSADGTYELKINNLTESDQGEYVCEISGEGGTSKTNLQFMGQAFKSIHEKVSKISETKKSDQKTTESTVTRKTEPKAPEPISSKPVIVTGLQDTTVSSDSVAKFAVKATGEPRPTAIWTKDGKAITQGGKYKLSEDKGGFFLEIHKTDTSDSGLYTCTVKNSAGSVSSSCKLTIKAIKDTEAQKVSTQKTSEITPQKKAVVQEEISQKALRSEEIKMSEAKSQEKLALKEEASKVLISEEVKKSAATSLEKSIVHEEITKTSQASEEVRTHAEIKAFSTQMSINEGQRLVLKANIAGATDVKWVLNGVELTNSEEYRYGVSGSDQTLTIKQASHRDEGILTCISKTKEGIVKCQYDLTLSKELSDAPAFISQPRSQNINEGQNVLFTCEISGEPSPEIEWFKNNLPISISSNVSISRSRNVYSLEIRNASVSDSGKYTIKAKNFRGQCSATASLMVLPLVEEPSREVVLRTSGDTSLQGSFSSQSVQMSASKQEASFSSFSSSSASSMTEMKFASMSAQSMSSMQESFVEMSSSSFMGISNMTQLESSTSKMLKAGIRGIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 2 - protein, 26927 aa, Homo sapiens
MTTQAPTFTQPLQSVVVLEGSTATFEAHISGFPVPEVSWFRDGQVISTSTLPGVQISFSDGRAKLTIPAVTKANSGRYSLKATNGSGQATSTAELLVKAETAPPNFVQRLQSMTVRQGSQVRLQVRVTGIPTPVVKFYRDGAEIQSSLDFQISQEGDLYSLLIAEAYPEDSGTYSVNATNSVGRATSTAELLVQGEEEVPAKKTKTIVSTAQISESRQTRIEKKIEAHFDARSIATVEMVIDGAAGQQLPHKTPPRIPPKPKSRSPTPPSIAAKAQLARQQSPSPIRHSPSPVRHVRAPTPSPVRSVSPAARISTSPIRSVRSPLLMRKTQASTVATGPEVPPPWKQEGYVASSSEAEMRETTLTTSTQIRTEERWEGRYGVQEQVTISGAAGAAASVSASASYAAEAVATGAKEVKQDADKSAAVATVVAAVDMARVREPVISAVEQTAQRTTTTAVHIQPAQEQVRKEAEKTAVTKVVVAADKAKEQELKSRTKEVITTKQEQMHVTHEQIRKETEKTFVPKVVISAAKAKEQETRISEEITKKQKQVTQEAIMKETRKTVVPKVIVATPKVKEQDLVSRGREGITTKREQVQITQEKMRKEAEKTALSTIAVATAKAKEQETILRTRETMATRQEQIQVTHGKVDVGKKAEAVATVVAAVDQARVREPREPGHLEESYAQQTTLEYGYKERISAAKVAEPPQRPASEPHVVPKAVKPRVIQAPSETHIKTTDQKGMHISSQIKKTTDLTTERLVHVDKRPRTASPHFTVSKISVPKTEHGYEASIAGSAIATLQKELSATSSAQKITKSVKAPTVKPSETRVRAEPTPLPQFPFADTPDTYKSEAGVEVKKEVGVSITGTTVREERFEVLHGREAKVTETARVPAPVEIPVTPPTLVVSGLKNVTVIEGESVTLECHISGYPSPTVTWYREDYQIESSIDFQITFQSGIARLMIREAFAEDSGRFTCSAVNEAGTVSTSCYLAVQVSEEFEKETTAVTEKFTTEEKRFVESRDVVMTDTSLTEEQAGPGEPAAPYFITKPVVQKLVEGGSVVFGCQVGGNPKPHVYWKKSGVPLTTGYRYKVSYNKQTGECKLVISMTFADDAGEYTIVVRNKHGETSASASLLEEADYELLMKSQQEMLYQTQVTAFVQEPKVGETAPGFVYSEYEKEYEKEQALIRKKMAKDTVVVRTYVEDQEFHISSFEERLIKEIEYRIIKTTLEELLEEDGEEKMAVDISESEAVESGFDSRIKNYRILEGMGVTFHCKMSGYPLPKIAWYKDGKRIKHGERYQMDFLQDGRASLRIPVVLPEDEGIYTAFASNIKGNAICSGKLYVEPAAPLGAPTYIPTLEPVSRIRSLSPRSVSRSPIRMSPARMSPARMSPARMSPARMSPGRRLEETDESQLERLYKPVFVLKPVSFKCLEGQTARFDLKVVGRPMPETFWFHDGQQIVNDYTHKVVIKEDGTQSLIIVPATPSDSGEWTVVAQNRAGRSSISVILTVEAVEHQVKPMFVEKLKNVNIKEGSRLEMKVRATGNPNPDIVWLKNSDIIVPHKYPKIRIEGTKGEAALKIDSTVSQDSAWYTATAINKAGRDTTRCKVNVEVEFAEPEPERKLIIPRGTYRAKEIAAPELEPLHLRYGQEQWEEGDLYDKEKQQKPFFKKKLTSLRLKRFGPAHFECRLTPIGDPTMVVEWLHDGKPLEAANRLRMINEFGYCSLDYGVAYSRDSGIITCRATNKYGTDHTSATLIVKDEKSLVEESQLPEGRKGLQRIEELERMAHEGALTGVTTDQKEKQKPDIVLYPEPVRVLEGETARFRCRVTGYPQPKVNWYLNGQLIRKSKRFRVRYDGIHYLDIVDCKSYDTGEVKVTAENPEGVIEHKVKLEIQQREDFRSVLRRAPEPRPEFHVHEPGKLQFEVQKVDRPVDTTETKEVVKLKRAERITHEKVPEESEELRSKFKRRTEEGYYEAITAVELKSRKKDESYEELLRKTKDELLHWTKELTEEEKKALAEEGKITIPTFKPDKIELSPSMEAPKIFERIQSQTVGQGSDAHFRVRVVGKPDPECEWYKNGVKIERSDRIYWYWPEDNVCELVIRDVTAEDSASIMVKAINIAGETSSHAFLLVQAKQLITFTQELQDVVAKEKDTMATFECETSEPFVKVKWYKDGMEVHEGDKYRMHSDRKVHFLSILTIDTSDAEDYSCVLVEDENVKTTAKLIVEGAVVEFVKELQDIEVPESYSGELECIVSPENIEGKWYHNDVELKSNGKYTITSRRGRQNLTVKDVTKEDQGEYSFVIDGKKTTCKLKMKPRPIAILQGLSDQKVCEGDIVQLEVKVSLESVEGVWMKDGQEVQPSDRVHIVIDKQSHMLLIEDMTKEDAGNYSFTIPALGLSTSGRVSVYSVDVITPLKDVNVIEGTKAVLECKVSVPDVTSVKWYLNDEQIKPDDRVQAIVKGTKQRLVINRTHASDEGPYKLIVGRVETNCNLSVEKIKIIRGLRDLTCTETQNVVFEVELSHSGIDVLWNFKDKEIKPSSKYKIEAHGKIYKLTVLNMMKDDEGKYTFYAGENMTSGKLTVAGGAISKPLTDQTVAESQEAVFECEVANPDSKGEWLRDGKHLPLTNNIRSESDGHKRRLIIAATKLDDIGEYTYKVATSKTSAKLKVEAVKIKKTLKNLTVTETQDAVFTVELTHPNVKGVQWIKNGVVLESNEKYAISVKGTIYSLRIKNCAIVDESVYGFRLGRLGASARLHVETVKIIKKPKDVTALENATVAFEVSVSHDTVPVKWFHKSVEIKPSDKHRLVSERKVHKLMLQNISPSDAGEYTAVVGQLECKAKLFVETLHITKTMKNIEVPETKTASFECEVSHFNVPSMWLKNGVEIEMSEKFKIVVQGKLHQLIIMNTSTEDSAEYTFVCGNDQVSATLTVTPIMITSMLKDINAEEKDTITFEVTVNYEGISYKWLKNGVEIKSTDKCQMRTKKLTHSLNIRNVHFGDAADYTFVAGKATSTATLYVEARHIEFRKHIKDIKVLEKKRAMFECEVSEPDITVQWMKDDQELQITDRIKIQKEKYVHRLLIPSTRMSDAGKYTVVAGGNVSTAKLFVEGRDVRIRSIKKEVQVIEKQRAVVEFEVNEDDVDAHWYKDGIEINFQVQERHKYVVERRIHRMFISETRQSDAGEYTFVAGRNRSSVTLYVNAPEPPQVLQELQPVTVQSGKPARFCAVISGRPQPKISWYKEEQLLSTGFKCKFLHDGQEYTLLLIEAFPEDAAVYTCEAKNDYGVATTSASLSVEVPEVVSPDQEMPVYPPAIITPLQDTVTSEGQPARFQCRVSGTDLKVSWYSKDKKIKPSRFFRMTQFEDTYQLEIAEAYPEDEGTYTFVASNAVGQVSSTANLSLEAPESILHERIEQEIEMEMKEFSSSFLSAEEEGLHSAELQLSKINETLELLSESPVYPTKFDSEKEGTGPIFIKEVSNADISMGDVATLSVTVIGIPKPKIQWFFNGVLLTPSADYKFVFDGDDHSLIILFTKLEDEGEYTCMASNDYGKTICSAYLKINSKGEGHKDTETESAVAKSLEKLGGPCPPHFLKELKPIRCAQGLPAIFEYTVVGEPAPTVTWFKENKQLCTSVYYTIIHNPNGSGTFIVNDPQREDSGLYICKAENMLGESTCAAELLVLLEDTDMTDTPCKAKSTPEAPEDFPQTPLKGPAVEALDSEQEIATFVKDTILKAALITEENQQLSYEHIAKANELSSQLPLGAQELQSILEQDKLTPESTREFLCINGSIHFQPLKEPSPNLQLQIVQSQKTFSKEGILMPEEPETQAVLSDTEKIFPSAMSIEQINSLTVEPLKTLLAEPEGNYPQSSIEPPMHSYLTSVAEEVLSPKEKTVSDTNREQRVTLQKQEAQSALILSQSLAEGHVESLQSPDVMISQVNYEPLVPSEHSCTEGGKILIESANPLENAGQDSAVRIEEGKSLRFPLALEEKQVLLKEEHSDNVVMPPDQIIESKREPVAIKKVQEVQGRDLLSKESLLSGIPEEQRLNLKIQICRALQAAVASEQPGLFSEWLRNIEKVEVEAVNITQEPRHIMCMYLVTSAKSVTEEVTIIIEDVDPQMANLKMELRDALCAIIYEEIDILTAEGPRIQQGAKTSLQEEMDSFSGSQKVEPITEPEVESKYLISTEEVSYFNVQSRVKYLDATPVTKGVASAVVSDEKQDESLKPSEEKEESSSESGTEEVATVKIQEAEGGLIKEDGPMIHTPLVDTVSEEGDIVHLTTSITNAKEVNWYFENKLVPSDEKFKCLQDQNTYTLVIDKVNTEDHQGEYVCEALNDSGKTATSAKLTVVKRAAPVIKRKIEPLEVALGHLAKFTCEIQSAPNVRFQWFKAGREIYESDKCSIRSSKYISSLEILRTQVVDCGEYTCKASNEYGSVSCTATLTVTVPGGEKKVRKLLPERKPEPKEEVVLKSVLRKRPEEEEPKVEPKKLEKVKKPAVPEPPPPKPVEEVEVPTVTKRERKIPEPTKVPEIKPAIPLPAPEPKPKPEAEVKTIKPPPVEPEPTPIAAPVTVPVVGKKAEAKAPKEEAAKPKGPIKGVPKKTPSPIEAERRKLRPGSGGEKPPDEAPFTYQLKAVPLKFVKEIKDIILTESEFVGSSAIFECLVSPSTAITTWMKDGSNIRESPKHRFIADGKDRKLHIIDVQLSDAGEYTCVLRLGNKEKTSTAKLVVEELPVRFVKTLEEEVTVVKGQPLYLSCELNKERDVVWRKDGKIVVEKPGRIVPGVIGLMRALTINDADDTDAGTYTVTVENANNLECSSCVKVVEVIRDWLVKPIRDQHVKPKGTAIFACDIAKDTPNIKWFKGYDEIPAEPNDKTEILRDGNHLYLKIKNAMPEDIAEYAVEIEGKRYPAKLTLGEREVELLKPIEDVTIYEKESASFDAEISEADIPGQWKLKGELLRPSPTCEIKAEGGKRFLTLHKVKLDQAGEVLYQALNAITTAILTVKEIELDFAVPLKDVTVPERRQARFECVLTREANVIWSKGPDIIKSSDKFDIIADGKKHILVINDSQFDDEGVYTAEVEGKKTSARLFVTGIRLKFMSPLEDQTVKEGETATFVCELSHEKMHVVWFKNDAKLHTSRTVLISSEGKTHKLEMKEVTLDDISQIKAQVKELSSTAQLKVLEADPYFTVKLHDKTAVEKDEITLKCEVSKDVPVKWFKDGEEIVPSPKYSIKADGLRRILKIKKADLKDKGEYVCDCGTDKTKANVTVEARLIKVEKPLYGVEVFVGETAHFEIELSEPDVHGQWKLKGQPLTASPDCEIIEDGKKHILILHNCQLGMTGEVSFQAANAKSAANLKVKELPLIFITPLSDVKVFEKDEAKFECEVSREPKTFRWLKGTQEITGDDRFELIKDGTKHSMVIKSAAFEDEAKYMFEAEDKHTSGKLIIEGIRLKFLTPLKDVTAKEKESAVFTVELSHDNIRVKWFKNDQRLHTTRSVSMQDEGKTHSITFKDLSIDDTSQIRVEAMGMSSEAKLTVLEGDPYFTGKLQDYTGVEKDEVILQCEISKADAPVKWFKDGKEIKPSKNAVIKADGKKRMLILKKALKSDIGQYTCDCGTDKTSGKLDIEDREIKLVRPLHSVEVMETETARFETEISEDDIHANWKLKGEALLQTPDCEIKEEGKIHSLVLHNCRLDQTGGVDFQAANVKSSAHLRVKPRVIGLLRPLKDVTVTAGETATFDCELSYEDIPVEWYLKGKKLEPSDKVVPRSEGKVHTLTLRDVKLEDAGEVQLTAKDFKTHANLFVKEPPVEFTKPLEDQTVEEGATAVLECEVSRENAKVKWFKNGTEILKSKKYEIVADGRVRKLVIHDCTPEDIKTYTCDAKDFKTSCNLNVVPPHVEFLRPLTDLQVREKEMARFECELSRENAKVKWFKDGAEIKKGKKYDIISKGAVRILVINKCLLDDEAEYSCEVRTARTSGMLTVLEEEAVFTKNLANIEVSETDTIKLVCEVSKPGAEVIWYKGDEEIIETGRYEILTEGRKRILVIQNAHLEDAGNYNCRLPSSRTDGKVKVHELAAEFISKPQNLEILEGEKAEFVCSISKESFPVQWKRDDKTLESGDKYDVIADGKKRVLVVKDATLQDMGTYVVMVGAARAAAHLTVIEKLRIVVPLKDTRVKEQQEVVFNCEVNTEGAKAKWFRNEEAIFDSSKYIILQKDLVYTLRIRDAHLDDQANYNVSLTNHRGENVKSAANLIVEEEDLRIVEPLKDIETMEKKSVTFWCKVNRLNVTLKWTKNGEEVPFDNRVSYRVDKYKHMLTIKDCGFPDEGEYIVTAGQDKSVAELLIIEAPTEFVEHLEDQTVTEFDDAVFSCQLSREKANVKWYRNGREIKEGKKYKFEKDGSIHRLIIKDCRLDDECEYACGVEDRKSRARLFVEEIPVEIIRPPQDILEAPGADVVFLAELNKDKVEVQWLRNNMVVVQGDKHQMMSEGKIHRLQICDIKPRDQGEYRFIAKDKEARAKLELAAAPKIKTADQDLVVDVGKPLTMVVPYDAYPKAEAEWFKENEPLSTKTIDTTAEQTSFRILEAKKGDKGRYKIVLQNKHGKAEGFINLKVIDVPGPVRNLEVTETFDGEVSLAWEEPLTDGGSKIIGYVVERRDIKRKTWVLATDRAESCEFTVTGLQKGGVEYLFRVSARNRVGTGEPVETDNPVEARSKYDVPGPPLNVTITDVNRFGVSLTWEPPEYDGGAEITNYVIELRDKTSIRWDTAMTVRAEDLSATVTDVVEGQEYSFRVRAQNRIGVGKPSAATPFVKVADPIERPSPPVNLTSSDQTQSSVQLKWEPPLKDGGSPILGYIIERCEEGKDNWIRCNMKLVPELTYKVTGLEKGNKYLYRVSAENKAGVSDPSEILGPLTADDAFVEPTMDLSAFKDGLEVIVPNPITILVPSTGYPRPTATWCFGDKVLETGDRVKMKTLSAYAELVISPSERSDKGIYTLKLENRVKTISGEIDVNVIARPSAPKELKFGDITKDSVHLTWEPPDDDGGSPLTGYVVEKREVSRKTWTKVMDFVTDLEFTVPDLVQGKEYLFKVCARNKCGPGEPAYVDEPVNMSTPATVPDPPENVKWRDRTANSIFLTWDPPKNDGGSRIKGYIVERCPRGSDKWVACGEPVAETKMEVTGLEEGKWYAYRVKALNRQGASKPSRPTEEIQAVDTQEAPEIFLDVKLLAGLTVKAGTKIELPATVTGKPEPKITWTKADMILKQDKRITIENVPKKSTVTIVDSKRSDTGTYIIEAVNVCGRATAVVEVNVLDKPGPPAAFDITDVTNESCLLTWNPPRDDGGSKITNYVVERRATDSEVWHKLSSTVKDTNFKATKLIPNKEYIFRVAAENMYGVGEPVQASPITAKYQFDPPGPPTRLEPSDITKDAVTLTWCEPDDDGGSPITGYWVERLDPDTDKWVRCNKMPVKDTTYRVKGLTNKKKYRFRVLAENLAGPGKPSKSTEPILIKDPIDPPWPPGKPTVKDVGKTSVRLNWTKPEHDGGAKIESYVIEMLKTGTDEWVRVAEGVPTTQHLLPGLMEGQEYSFRVRAVNKAGESEPSEPSDPVLCREKLYPPSPPRWLEVINITKNTADLKWTVPEKDGGSPITNYIVEKRDVRRKGWQTVDTTVKDTKCTVTPLTEGSLYVFRVAAENAIGQSDYTEIEDSVLAKDTFTTPGPPYALAVVDVTKRHVDLKWEPPKNDGGRPIQRYVIEKKERLGTRWVKAGKTAGPDCNFRVTDVIEGTEVQFQVRAENEAGVGHPSEPTEILSIEDPTSPPSPPLDLHVTDAGRKHIAIAWKPPEKNGGSPIIGYHVEMCPVGTEKWMRVNSRPIKDLKFKVEEGVVPDKEYVLRVRAVNAIGVSEPSEISENVVAKDPDCKPTIDLETHDIIVIEGEKLSIPVPFRAVPVPTVSWHKDGKEVKASDRLTMKNDHISAHLEVPKSVRADAGIYTITLENKLGSATASINVKVIGLPGPCKDIKASDITKSSCKLTWEPPEFDGGTPILHYVLERREAGRRTYIPVMSGENKLSWTVKDLIPNGEYFFRVKAVNKVGGGEYIELKNPVIAQDPKQPPDPPVDVEVHNPTAEAMTITWKPPLYDGGSKIMGYIIEKIAKGEERWKRCNEHLVPILTYTAKGLEEGKEYQFRVRAENAAGISEPSRATPPTKAVDPIDAPKVILRTSLEVKRGDEIALDASISGSPYPTITWIKDENVIVPEEIKKRAAPLVRRRKGEVQEEEPFVLPLTQRLSIDNSKKGESQLRVRDSLRPDHGLYMIKVENDHGIAKAPCTVSVLDTPGPPINFVFEDIRKTSVLCKWEPPLDDGGSEIINYTLEKKDKTKPDSEWIVVTSTLRHCKYSVTKLIEGKEYLFRVRAENRFGPGPPCVSKPLVAKDPFGPPDAPDKPIVEDVTSNSMLVKWNEPKDNGSPILGYWLEKREVNSTHWSRVNKSLLNALKANVDGLLEGLTYVFRVCAENAAGPGKFSPPSDPKTAHDPISPPGPPIPRVTDTSSTTIELEWEPPAFNGGGEIVGYFVDKQLVGTNEWSRCTEKMIKVRQYTVKEIREGADYKLRVSAVNAAGEGPPGETQPVTVAEPQEPPAVELDVSVKGGIQIMAGKTLRIPAVVTGRPVPTKVWTKEEGELDKDRVVIDNVGTKSELIIKDALRKDHGRYVITATNSCGSKFAAARVEVFDVPGPVLDLKPVVTNRKMCLLNWSDPEDDGGSEITGFIIERKDAKMHTWRQPIETERSKCDITGLLEGQEYKFRVIAKNKFGCGPPVEIGPILAVDPLGPPTSPERLTYTERTKSTITLDWKEPRSNGGSPIQGYIIEKRRHDKPDFERVNKRLCPTTSFLVENLDEHQMYEFRVKAVNEIGESEPSLPLNVVIQDDEVPPTIKLRLSVRGDTIKVKAGEPVHIPADVTGLPMPKIEWSKNETVIEKPTDALQITKEEVSRSEAKTELSIPKAVREDKGTYTVTASNRLGSVFRNVHVEVYDRPSPPRNLAVTDIKAESCYLTWDAPLDNGGSEITHYVIDKRDASRKKAEWEEVTNTAVEKRYGIWKLIPNGQYEFRVRAVNKYGISDECKSDKVVIQDPYRLPGPPGKPKVLARTKGSMLVSWTPPLDNGGSPITGYWLEKREEGSPYWSRVSRAPITKVGLKGVEFNVPRLLEGVKYQFRAMAINAAGIGPPSEPSDPEVAGDPIFPPGPPSCPEVKDKTKSSISLGWKPPAKDGGSPIKGYIVEMQEEGTTDWKRVNEPDKLITTCECVVPNLKELRKYRFRVKAVNEAGESEPSDTTGEIPATDIQEEPEVFIDIGAQDCLVCKAGSQIRIPAVIKGRPTPKSSWEFDGKAKKAMKDGVHDIPEDAQLETAENSSVIIIPECKRSHTGKYSITAKNKAGQKTANCRVKVMDVPGPPKDLKVSDITRGSCRLSWKMPDDDGGDRIKGYVIEKRTIDGKAWTKVNPDCGSTTFVVPDLLSEQQYFFRVRAENRFGIGPPVETIQRTTARDPIYPPDPPIKLKIGLITKNTVHLSWKPPKNDGGSPVTHYIVECLAWDPTGTKKEAWRQCNKRDVEELQFTVEDLVEGGEYEFRVKAVNAAGVSKPSATVGPVTVKDQTCPPSIDLKEFMEVEEGTNVNIVAKIKGVPFPTLTWFKAPPKKPDNKEPVLYDTHVNKLVVDDTCTLVIPQSRRSDTGLYTITAVNNLGTASKEMRLNVLGRPGPPVGPIKFESVSADQMTLSWFPPKDDGGSKITNYVIEKREANRKTWVHVSSEPKECTYTIPKLLEGHEYVFRIMAQNKYGIGEPLDSEPETARNLFSVPGAPDKPTVSSVTRNSMTVNWEEPEYDGGSPVTGYWLEMKDTTSKRWKRVNRDPIKAMTLGVSYKVTGLIEGSDYQFRVYAINAAGVGPASLPSDPATARDPIAPPGPPFPKVTDWTKSSADLEWSPPLKDGGSKVTGYIVEYKEEGKEEWEKGKDKEVRGTKLVVTGLKEGAFYKFRVRAVNIAGIGEPGEVTDVIEMKDRLVSPDLQLDASVRDRIVVHAGGVIRIIAYVSGKPPPTVTWNMNERTLPQEATIETTAISSSMVIKNCQRSHQGVYSLLAKNEAGERKKTIIVDVLDVPGPVGTPFLAHNLTNESCKLTWFSPEDDGGSPITNYVIEKRESDRRAWTPVTYTVTRQNATVQGLIQGKAYFFRIAAENSIGMGPFVETSEALVIREPITVPERPEDLEVKEVTKNTVTLTWNPPKYDGGSEIINYVLESRLIGTEKFHKVTNDNLLSRKYTVKGLKEGDTYEYRVSAVNIVGQGKPSFCTKPITCKDELAPPTLHLDFRDKLTIRVGEAFALTGRYSGKPKPKVSWFKDEADVLEDDRTHIKTTPATLALEKIKAKRSDSGKYCVVVENSTGSRKGFCQVNVVDRPGPPVGPVSFDEVTKDYMVISWKPPLDDGGSKITNYIIEKKEVGKDVWMPVTSASAKTTCKVSKLLEGKDYIFRIHAENLYGISDPLVSDSMKAKDRFRVPDAPDQPIVTEVTKDSALVTWNKPHDGGKPITNYILEKRETMSKRWARVTKDPIHPYTKFRVPDLLEGCQYEFRVSAENEIGIGDPSPPSKPVFAKDPIAKPSPPVNPEAIDTTCNSVDLTWQPPRHDGGSKILGYIVEYQKVGDEEWRRANHTPESCPETKYKVTGLRDGQTYKFRVLAVNAAGESDPAHVPEPVLVKDRLEPPELILDANMAREQHIKVGDTLRLSAIIKGVPFPKVTWKKEDRDAPTKARIDVTPVGSKLEIRNAAHEDGGIYSLTVENPAGSKTVSVKVLVLDKPGPPRDLEVSEIRKDSCYLTWKEPLDDGGSVITNYVVERRDVASAQWSPLSATSKKKSHFAKHLNEGNQYLFRVAAENQYGRGPFVETPKPIKALDPLHPPGPPKDLHHVDVDKTEVSLVWNKPDRDGGSPITGYLVEYQEEGTQDWIKFKTVTNLECVVTGLQQGKTYRFRVKAENIVGLGLPDTTIPIECQEKLVPPSVELDVKLIEGLVVKAGTTVRFPAIIRGVPVPTAKWTTDGSEIKTDEHYTVETDNFSSVLTIKNCLRRDTGEYQITVSNAAGSKTVAVHLTVLDVPGPPTGPINILDVTPEHMTISWQPPKDDGGSPVINYIVEKQDTRKDTWGVVSSGSSKTKLKIPHLQKGCEYVFRVRAENKIGVGPPLDSTPTVAKHKFSPPSPPGKPVVTDITENAATVSWTLPKSDGGSPITGYYMERREVTGKWVRVNKTPIADLKFRVTGLYEGNTYEFRVFAENLAGLSKPSPSSDPIKACRPIKPPGPPINPKLKDKSRETADLVWTKPLSDGGSPILGYVVECQKPGTAQWNRINKDELIRQCAFRVPGLIEGNEYRFRIKAANIVGEGEPRELAESVIAKDILHPPEVELDVTCRDVITVRVGQTIRILARVKGRPEPDITWTKEGKVLVREKRVDLIQDLPRVELQIKEAVRADHGKYIISAKNSSGHAQGSAIVNVLDRPGPCQNLKVTNVTKENCTISWENPLDNGGSEITNFIVEYRKPNQKGWSIVASDVTKRLIKANLLANNEYYFRVCAENKVGVGPTIETKTPILAINPIDRPGEPENLHIADKGKTFVYLKWRRPDYDGGSPNLSYHVERRLKGSDDWERVHKGSIKETHYMVDRCVENQIYEFRVQTKNEGGESDWVKTEEVVVKEDLQKPVLDLKLSGVLTVKAGDTIRLEAGVRGKPFPEVAWTKDKDATDLTRSPRVKIDTRADSSKFSLTKAKRSDGGKYVVTATNTAGSFVAYATVNVLDKPGPVRNLKIVDVSSDRCTVCWDPPEDDGGCEIQNYILEKCETKRMVWSTYSATVLTPGTTVTRLIEGNEYIFRVRAENKIGTGPPTESKPVIAKTKYDKPGRPDPPEVTKVSKEEMTVVWNPPEYDGGKSITGYFLEKKEKHSTRWVPVNKSAIPERRMKVQNLLPDHEYQFRVKAENEIGIGEPSLPSRPVVAKDPIEPPGPPTNFRVVDTTKHSITLGWGKPVYDGGAPIIGYVVEMRPKIADASPDEGWKRCNAAAQLVRKEFTVTSLDENQEYEFRVCAQNQVGIGRPAELKEAIKPKEILEPPEIDLDASMRKLVIVRAGCPIRLFAIVRGRPAPKVTWRKVGIDNVVRKGQVDLVDTMAFLVIPNSTRDDSGKYSLTLVNPAGEKAVFVNVRVLDTPGPVSDLKVSDVTKTSCHVSWAPPENDGGSQVTHYIVEKREADRKTWSTVTPEVKKTSFHVTNLVPGNEYYFRVTAVNEYGPGVPTDVPKPVLASDPLSEPDPPRKLEVTEMTKNSATLAWLPPLRDGGAKIDGYITSYREEEQPADRWTEYSVVKDLSLVVTGLKEGKKYKFRVAARNAVGVSLPREAEGVYEAKEQLLPPKILMPEQITIKAGKKLRIEAHVYGKPHPTCKWKKGEDEVVTSSHLAVHKADSSSILIIKDVTRKDSGYYSLTAENSSGTDTQKIKVVVMDAPGPPQPPFDISDIDADACSLSWHIPLEDGGSNITNYIVEKCDVSRGDWVTALASVTKTSCRVGKLIPGQEYIFRVRAENRFGISEPLTSPKMVAQFPFGVPSEPKNARVTKVNKDCIFVAWDRPDSDGGSPIIGYLIERKERNSLLWVKANDTLVRSTEYPCAGLVEGLEYSFRIYALNKAGSSPPSKPTEYVTARMPVDPPGKPEVIDVTKSTVSLIWARPKHDGGSKIIGYFVEACKLPGDKWVRCNTAPHQIPQEEYTATGLEEKAQYQFRAIARTAVNISPPSEPSDPVTILAENVPPRIDLSVAMKSLLTVKAGTNVCLDATVFGKPMPTVSWKKDGTLLKPAEGIKMAMQRNLCTLELFSVNRKDSGDYTITAENSSGSKSATIKLKVLDKPGPPASVKINKMYSDRAMLSWEPPLEDGGSEITNYIVDKRETSRPNWAQVSATVPITSCSVEKLIEGHEYQFRICAENKYGVGDPVFTEPAIAKNPYDPPGRCDPPVISNITKDHMTVSWKPPADDGGSPITGYLLEKRETQAVNWTKVNRKPIIERTLKATGLQEGTEYEFRVTAINKAGPGKPSDASKAAYARDPQYPPGPPAFPKVYDTTRSSVSLSWGKPAYDGGSPIIGYLVEVKRADSDNWVRCNLPQNLQKTRFEVTGLMEDTQYQFRVYAVNKIGYSDPSDVPDKHYPKDILIPPEGELDADLRKTLILRAGVTMRLYVPVKGRPPPKITWSKPNVNLRDRIGLDIKSTDFDTFLRCENVNKYDAGKYILTLENSCGKKEYTIVVKVLDTPGPPVNVTVKEISKDSAYVTWEPPIIDGGSPIINYVVQKRDAERKSWSTVTTECSKTSFRVANLEEGKSYFFRVFAENEYGIGDPGETRDAVKASQTPGPVVDLKVRSVSKSSCSIGWKKPHSDGGSRIIGYVVDFLTEENKWQRVMKSLSLQYSAKDLTEGKEYTFRVSAENENGEGTPSEITVVARDDVVAPDLDLKGLPDLCYLAKENSNFRLKIPIKGKPAPSVSWKKGEDPLATDTRVSVESSAVNTTLIVYDCQKSDAGKYTITLKNVAGTKEGTISIKVVGKPGIPTGPIKFDEVTAEAMTLKWAPPKDDGGSEITNYILEKRDSVNNKWVTCASAVQKTTFRVTRLHEGMEYTFRVSAENKYGVGEGLKSEPIVARHPFDVPDAPPPPNIVDVRHDSVSLTWTDPKKTGGSPITGYHLEFKERNSLLWKRANKTPIRMRDFKVTGLTEGLEYEFRVMAINLAGVGKPSLPSEPVVALDPIDPPGKPEVINITRNSVTLIWTEPKYDGGHKLTGYIVEKRDLPSKSWMKANHVNVPECAFTVTDLVEGGKYEFRIRAKNTAGAISAPSESTETIICKDEYEAPTIVLDPTIKDGLTIKAGDTIVLNAISILGKPLPKSSWSKAGKDIRPSDITQITSTPTSSMLTIKYATRKDAGEYTITATNPFGTKVEHVKVTVLDVPGPPGPVEISNVSAEKATLTWTPPLEDGGSPIKSYILEKRETSRLLWTVVSEDIQSCRHVATKLIQGNEYIFRVSAVNHYGKGEPVQSEPVKMVDRFGPPGPPEKPEVSNVTKNTATVSWKRPVDDGGSEITGYHVERREKKSLRWVRAIKTPVSDLRCKVTGLQEGSTYEFRVSAENRAGIGPPSEASDSVLMKDAAYPPGPPSNPHVTDTTKKSASLAWGKPHYDGGLEITGYVVEHQKVGDEAWIKDTTGTALRITQFVVPDLQTKEKYNFRISAINDAGVGEPAVIPDVEIVEREMAPDFELDAELRRTLVVRAGLSIRIFVPIKGRPAPEVTWTKDNINLKNRANIENTESFTLLIIPECNRYDTGKFVMTIENPAGKKSGFVNVRVLDTPGPVLNLRPTDITKDSVTLHWDLPLIDGGSRITNYIVEKREATRKSYSTATTKCHKCTYKVTGLSEGCEYFFRVMAENEYGIGEPTETTEPVKASEAPSPPDSLNIMDITKSTVSLAWPKPKHDGGSKITGYVIEAQRKGSDQWTHITTVKGLECVVRNLTEGEEYTFQVMAVNSAGRSAPRESRPVIVKEQTMLPELDLRGIYQKLVIAKAGDNIKVEIPVLGRPKPTVTWKKGDQILKQTQRVNFETTATSTILNINECVRSDSGPYPLTARNIVGEVGDVITIQVHDIPGPPTGPIKFDEVSSDFVTFSWDPPENDGGVPISNYVVEMRQTDSTTWVELATTVIRTTYKATRLTTGLEYQFRVKAQNRYGVGPGITSACIVANYPFKVPGPPGTPQVTAVTKDSMTISWHEPLSDGGSPILGYHVERKERNGILWQTVSKALVPGNIFKSSGLTDGIAYEFRVIAENMAGKSKPSKPSEPMLALDPIDPPGKPVPLNITRHTVTLKWAKPEYTGGFKITSYIVEKRDLPNGRWLKANFSNILENEFTVSGLTEDAAYEFRVIAKNAAGAISPPSEPSDAITCRDDVEAPKIKVDVKFKDTVILKAGEAFRLEADVSGRPPPTMEWSKDGKELEGTAKLEIKIADFSTNLVNKDSTRRDSGAYTLTATNPGGFAKHIFNVKVLDRPGPPEGPLAVTEVTSEKCVLSWFPPLDDGGAKIDHYIVQKRETSRLAWTNVASEVQVTKLKVTKLLKGNEYIFRVMAVNKYGVGEPLESEPVLAVNPYGPPDPPKNPEVTTITKDSMVVCWGHPDSDGGSEIINYIVERRDKAGQRWIKCNKKTLTDLRYKVSGLTEGHEYEFRIMAENAAGISAPSPTSPFYKACDTVFKPGPPGNPRVLDTSRSSISIAWNKPIYDGGSEITGYMVEIALPEEDEWQIVTPPAGLKATSYTITGLTENQEYKIRIYAMNSEGLGEPALVPGTPKAEDRMLPPEIELDADLRKVVTIRACCTLRLFVPIKGRPAPEVKWARDHGESLDKASIESTSSYTLLIVGNVNRFDSGKYILTVENSSGSKSAFVNVRVLDTPGPPQDLKVKEVTKTSVTLTWDPPLLDGGSKIKNYIVEKRESTRKAYSTVATNCHKTSWKVDQLQEGCSYYFRVLAENEYGIGLPAETAESVKASERPLPPGKITLMDVTRNSVSLSWEKPEHDGGSRILGYIVEMQTKGSDKWATCATVKVTEATITGLIQGEEYSFRVSAQNEKGISDPRQLSVPVIAKDLVIPPAFKLLFNTFTVLAGEDLKVDVPFIGRPTPAVTWHKDNVPLKQTTRVNAESTENNSLLTIKDACREDVGHYVVKLTNSAGEAIETLNVIVLDKPGPPTGPVKMDEVTADSITLSWGPPKYDGGSSINNYIVEKRDTSTTTWQIVSATVARTTIKACRLKTGCEYQFRIAAENRYGKSTYLNSEPTVAQYPFKVPGPPGTPVVTLSSRDSMEVQWNEPISDGGSRVIGYHLERKERNSILWVKLNKTPIPQTKFKTTGLEEGVEYEFRVSAENIVGIGKPSKVSECYVARDPCDPPGRPEAIIVTRNSVTLQWKKPTYDGGSKITGYIVEKKELPEGRWMKASFTNIIDTHFEVTGLVEDHRYEFRVIARNAAGVFSEPSESTGAITARDEVDPPRISMDPKYKDTIVVHAGESFKVDADIYGKPIPTIQWIKGDQELSNTARLEIKSTDFATSLSVKDAVRVDSGNYILKAKNVAGERSVTVNVKVLDRPGPPEGPVVISGVTAEKCTLAWKPPLQDGGSDIINYIVERRETSRLVWTVVDANVQTLSCKVTKLLEGNEYTFRIMAVNKYGVGEPLESEPVVAKNPFVVPDAPKAPEVTTVTKDSMIVVWERPASDGGSEILGYVLEKRDKEGIRWTRCHKRLIGELRLRVTGLIENHDYEFRVSAENAAGLSEPSPPSAYQKACDPIYKPGPPNNPKVIDITRSSVFLSWSKPIYDGGCEIQGYIVEKCDVSVGEWTMCTPPTGINKTNIEVEKLLEKHEYNFRICAINKAGVGEHADVPGPIIVEEKLEAPDIDLDLELRKIINIRAGGSLRLFVPIKGRPTPEVKWGKVDGEIRDAAIIDVTSSFTSLVLDNVNRYDSGKYTLTLENSSGTKSAFVTVRVLDTPSPPVNLKVTEITKDSVSITWEPPLLDGGSKIKNYIVEKREATRKSYAAVVTNCHKNSWKIDQLQEGCSYYFRVTAENEYGIGLPAQTADPIKVAEVPQPPGKITVDDVTRNSVSLSWTKPEHDGGSKIIQYIVEMQAKHSEKWSECARVKSLQAVITNLTQGEEYLFRVVAVNEKGRSDPRSLAVPIVAKDLVIEPDVKPAFSSYSVQVGQDLKIEVPISGRPKPTITWTKDGLPLKQTTRINVTDSLDLTTLSIKETHKDDGGQYGITVANVVGQKTASIEIVTLDKPDPPKGPVKFDDVSAESITLSWNPPLYTGGCQITNYIVQKRDTTTTVWDVVSATVARTTLKVTKLKTGTEYQFRIFAENRYGQSFALESDPIVAQYPYKEPGPPGTPFATAISKDSMVIQWHEPVNNGGSPVIGYHLERKERNSILWTKVNKTIIHDTQFKAQNLEEGIEYEFRVYAENIVGVGKASKNSECYVARDPCDPPGTPEPIMVKRNEITLQWTKPVYDGGSMITGYIVEKRDLPDGRWMKASFTNVIETQFTVSGLTEDQRYEFRVIAKNAAGAISKPSDSTGPITAKDEVELPRISMDPKFRDTIVVNAGETFRLEADVHGKPLPTIEWLRGDKEIEESARCEIKNTDFKALLIVKDAIRIDGGQYILRASNVAGSKSFPVNVKVLDRPGPPEGPVQVTGVTSEKCSLTWSPPLQDGGSDISHYVVEKRETSRLAWTVVASEVVTNSLKVTKLLEGNEYVFRIMAVNKYGVGEPLESAPVLMKNPFVLPGPPKSLEVTNIAKDSMTVCWNRPDSDGGSEIIGYIVEKRDRSGIRWIKCNKRRITDLRLRVTGLTEDHEYEFRVSAENAAGVGEPSPATVYYKACDPVFKPGPPTNAHIVDTTKNSITLAWGKPIYDGGSEILGYVVEICKADEEEWQIVTPQTGLRVTRFEISKLTEHQEYKIRVCALNKVGLGEATSVPGTVKPEDKLEAPELDLDSELRKGIVVRAGGSARIHIPFKGRPTPEITWSREEGEFTDKVQIEKGVNYTQLSIDNCDRNDAGKYILKLENSSGSKSAFVTVKVLDTPGPPQNLAVKEVRKDSAFLVWEPPIIDGGAKVKNYVIDKRESTRKAYANVSSKCSKTSFKVENLTEGAIYYFRVMAENEFGVGVPVETVDAVKAAEPPSPPGKVTLTDVSQTSASLMWEKPEHDGGSRVLGYVVEMQPKGTEKWSIVAESKVCNAVVTGLSSGQEYQFRVKAYNEKGKSDPRVLGVPVIAKDLTIQPSLKLPFNTYSIQAGEDLKIEIPVIGRPRPNISWVKDGEPLKQTTRVNVEETATSTVLHIKEGNKDDFGKYTVTATNSAGTATENLSVIVLEKPGPPVGPVRFDEVSADFVVISWEPPAYTGGCQISNYIVEKRDTTTTTWHMVSATVARTTIKITKLKTGTEYQFRIFAENRYGKSAPLDSKAVIVQYPFKEPGPPGTPFVTSISKDQMLVQWHEPVNDGGTKIIGYHLEQKEKNSILWVKLNKTPIQDTKFKTTGLDEGLEYEFKVSAENIVGIGKPSKVSECFVARDPCDPPGRPEAIVITRNNVTLKWKKPAYDGGSKITGYIVEKKDLPDGRWMKASFTNVLETEFTVSGLVEDQRYEFRVIARNAAGNFSEPSDSSGAITARDEIDAPNASLDPKYKDVIVVHAGETFVLEADIRGKPIPDVVWSKDGKELEETAARMEIKSTIQKTTLVVKDCIRTDGGQYILKLSNVGGTKSIPITVKVLDRPGPPEGPLKVTGVTAEKCYLAWNPPLQDGGANISHYIIEKRETSRLSWTQVSTEVQALNYKVTKLLPGNEYIFRVMAVNKYGIGEPLESGPVTACNPYKPPGPPSTPEVSAITKDSMVVTWARPVDDGGTEIEGYILEKRDKEGVRWTKCNKKTLTDLRLRVTGLTEGHSYEFRVAAENAAGVGEPSEPSVFYRACDALYPPGPPSNPKVTDTSRSSVSLAWSKPIYDGGAPVKGYVVEVKEAAADEWTTCTPPTGLQGKQFTVTKLKENTEYNFRICAINSEGVGEPATLPGSVVAQERIEPPEIELDADLRKVVVLRASATLRLFVTIKGRPEPEVKWEKAEGILTDRAQIEVTSSFTMLVIDNVTRFDSGRYNLTLENNSGSKTAFVNVRVLDSPSAPVNLTIREVKKDSVTLSWEPPLIDGGAKITNYIVEKRETTRKAYATITNNCTKTTFRIENLQEGCSYYFRVLASNEYGIGLPAETTEPVKVSEPPLPPGRVTLVDVTRNTATIKWEKPESDGGSKITGYVVEMQTKGSEKWSTCTQVKTLEATISGLTAGEEYVFRVAAVNEKGRSDPRQLGVPVIARDIEIKPSVELPFHTFNVKAREQLKIDVPFKGRPQATVNWRKDGQTLKETTRVNVSSSKTVTSLSIKEASKEDVGTYELCVSNSAGSITVPITIIVLDRPGPPGPIRIDEVSCDSITISWNPPEYDGGCQISNYIVEKKETTSTTWHIVSQAVARTSIKIVRLTTGSEYQFRVCAENRYGKSSYSESSAVVAEYPFSPPGPPGTPKVVHATKSTMLVTWQVPVNDGGSRVIGYHLEYKERSSILWSKANKILIADTQMKVSGLDEGLMYEYRVYAENIAGIGKCSKSCEPVPARDPCDPPGQPEVTNITRKSVSLKWSKPHYDGGAKITGYIVERRELPDGRWLKCNYTNIQETYFEVTELTEDQRYEFRVFARNAADSVSEPSESTGPIIVKDDVEPPRVMMDVKFRDVIVVKAGEVLKINADIAGRPLPVISWAKDGIEIEERARTEIISTDNHTLLTVKDCIRRDTGQYVLTLKNVAGTRSVAVNCKVLDKPGPPAGPLEINGLTAEKCSLSWGRPQEDGGADIDYYIVEKRETSHLAWTICEGELQMTSCKVTKLLKGNEYIFRVTGVNKYGVGEPLESVAIKALDPFTVPSPPTSLEITSVTKESMTLCWSRPESDGGSEISGYIIERREKNSLRWVRVNKKPVYDLRVKSTGLREGCEYEYRVYAENAAGLSLPSETSPLIRAEDPVFLPSPPSKPKIVDSGKTTITIAWVKPLFDGGAPITGYTVEYKKSDDTDWKTSIQSLRGTEYTISGLTTGAEYVFRVKSVNKVGASDPSDSSDPQIAKEREEEPLFDIDSEMRKTLIVKAGASFTMTVPFRGRPVPNVLWSKPDTDLRTRAYVDTTDSRTSLTIENANRNDSGKYTLTIQNVLSAASLTLVVKVLDTPGPPTNITVQDVTKESAVLSWDVPENDGGAPVKNYHIEKREASKKAWVSVTNNCNRLSYKVTNLQEGAIYYFRVSGENEFGVGIPAETKEGVKITEKPSPPEKLGVTSISKDSVSLTWLKPEHDGGSRIVHYVVEALEKGQKNWVKCAVAKSTHHVVSGLRENSEYFFRVFAENQAGLSDPRELLLPVLIKEQLEPPEIDMKNFPSHTVYVRAGSNLKVDIPISGKPLPKVTLSRDGVPLKATMRFNTEITAENLTINLKESVTADAGRYEITAANSSGTTKAFINIVVLDRPGPPTGPVVISDITEESVTLKWEPPKYDGGSQVTNYILLKRETSTAVWTEVSATVARTMMKVMKLTTGEEYQFRIKAENRFGISDHIDSACVTVKLPYTTPGPPSTPWVTNVTRESITVGWHEPVSNGGSAVVGYHLEMKDRNSILWQKANKLVIRTTHFKVTTISAGLIYEFRVYAENAAGVGKPSHPSEPVLAIDACEPPRNVRITDISKNSVSLSWQQPAFDGGSKITGYIVERRDLPDGRWTKASFTNVTETQFIISGLTQNSQYEFRVFARNAVGSISNPSEVVGPITCIDSYGGPVIDLPLEYTEVVKYRAGTSVKLRAGISGKPAPTIEWYKDDKELQTNALVCVENTTDLASILIKDADRLNSGCYELKLRNAMGSASATIRVQILDKPGPPGGPIEFKTVTAEKITLLWRPPADDGGAKITHYIVEKRETSRVVWSMVSEHLEECIITTTKIIKGNEYIFRVRAVNKYGIGEPLESDSVVAKNAFVTPGPPGIPEVTKITKNSMTVVWSRPIADGGSDISGYFLEKRDKKSLGWFKVLKETIRDTRQKVTGLTENSDYQYRVCAVNAAGQGPFSEPSEFYKAADPIDPPGPPAKIRIADSTKSSITLGWSKPVYDGGSAVTGYVVEIRQGEEEEWTTVSTKGEVRTTEYVVSNLKPGVNYYFRVSAVNCAGQGEPIEMNEPVQAKDILEAPEIDLDVALRTSVIAKAGEDVQVLIPFKGRPPPTVTWRKDEKNLGSDARYSIENTDSSSLLTIPQVTRNDTGKYILTIENGVGEPKSSTVSVKVLDTPAACQKLQVKHVSRGTVTLLWDPPLIDGGSPIINYVIEKRDATKRTWSVVSHKCSSTSFKLIDLSEKTPFFFRVLAENEIGIGEPCETTEPVKAAEVPAPIRDLSMKDSTKTSVILSWTKPDFDGGSVITEYVVERKGKGEQTWSHAGISKTCEIEVSQLKEQSVLEFRVFAKNEKGLSDPVTIGPITVKELIITPEVDLSDIPGAQVTVRIGHNVHLELPYKGKPKPSISWLKDGLPLKESEFVRFSKTENKITLSIKNAKKEHGGKYTVILDNAVCRIAVPITVITLGPPSKPKGPIRFDEIKADSVILSWDVPEDNGGGEITCYSIEKRETSQTNWKMVCSSVARTTFKVPNLVKDAEYQFRVRAENRYGVSQPLVSSIIVAKHQFRIPGPPGKPVIYNVTSDGMSLTWDAPVYDGGSEVTGFHVEKKERNSILWQKVNTSPISGREYRATGLVEGLDYQFRVYAENSAGLSSPSDPSKFTLAVSPVDPPGTPDYIDVTRETITLKWNPPLRDGGSKIVGYSIEKRQGNERWVRCNFTDVSECQYTVTGLSPGDRYEFRIIARNAVGTISPPSQSSGIIMTRDENVPPIVEFGPEYFDGLIIKSGESLRIKALVQGRPVPRVTWFKDGVEIEKRMNMEITDVLGSTSLFVRDATRDHRGVYTVEAKNASGSAKAEIKVKVQDTPGKVVGPIRFTNITGEKMTLWWDAPLNDGCAPITHYIIEKRETSRLAWALIEDKCEAQSYTAIKLINGNEYQFRVSAVNKFGVGRPLDSDPVVAQIQYTVPDAPGIPEPSNITGNSITLTWARPESDGGSEIQQYILERREKKSTRWVKVISKRPISETRFKVTGLTEGNEYEFHVMAENAAGVGPASGISRLIKCREPVNPPGPPTVVKVTDTSKTTVSLEWSKPVFDGGMEIIGYIIEMCKADLGDWHKVNAEACVKTRYTVTDLQAGEEYKFRVSAINGAGKGDSCEVTGTIKAVDRLTAPELDIDANFKQTHVVRAGASIRLFIAYQGRPTPTAVWSKPDSNLSLRADIHTTDSFSTLTVENCNRNDAGKYTLTVENNSGSKSITFTVKVLDTPGPPGPITFKDVTRGSATLMWDAPLLDGGARIHHYVVEKREASRRSWQVISEKCTRQIFKVNDLAEGVPYYFRVSAVNEYGVGEPYEMPEPIVATEQPAPPRRLDVVDTSKSSAVLAWLKPDHDGGSRITGYLLEMRQKGSDFWVEAGHTKQLTFTVERLVEKTEYEFRVKAKNDAGYSEPREAFSSVIIKEPQIEPTADLTGITNQLITCKAGSPFTIDVPISGRPAPKVTWKLEEMRLKETDRVSITTTKDRTTLTVKDSMRGDSGRYFLTLENTAGVKTFSVTVVVIGRPGPVTGPIEVSSVSAESCVLSWGEPKDGGGTEITNYIVEKRESGTTAWQLVNSSVKRTQIKVTHLTKYMEYSFRVSSENRFGVSKPLESAPIIAEHPFVPPSAPTRPEVYHVSANAMSIRWEEPYHDGGSKIIGYWVEKKERNTILWVKENKVPCLECNYKVTGLVEGLEYQFRTYALNAAGVSKASEASRPIMAQNPVDAPGRPEVTDVTRSTVSLIWSAPAYDGGSKVVGYIIERKPVSEVGDGRWLKCNYTIVSDNFFTVTALSEGDTYEFRVLAKNAAGVISKGSESTGPVTCRDEYAPPKAELDARLHGDLVTIRAGSDLVLDAAVGGKPEPKIIWTKGDKELDLCEKVSLQYTGKRATAVIKFCDRSDSGKYTLTVKNASGTKAVSVMVKVLDSPGPCGKLTVSRVTQEKCTLAWSLPQEDGGAEITHYIVERRETSRLNWVIVEGECPTLSYVVTRLIKNNEYIFRVRAVNKYGPGVPVESEPIVARNSFTIPSPPGIPEEVGTGKEHIIIQWTKPESDGGNEISNYLVDKREKKSLRWTRVNKDYVVYDTRLKVTSLMEGCDYQFRVTAVNAAGNSEPSEASNFISCREPSYTPGPPSAPRVVDTTKHSISLAWTKPMYDGGTDIVGYVLEMQEKDTDQWYRVHTNATIRNTEFTVPDLKMGQKYSFRVAAVNVKGMSEYSESIAEIEPVERIEIPDLELADDLKKTVTIRAGASLRLMVSVSGRPPPVITWSKQGIDLASRAIIDTTESYSLLIVDKVNRYDAGKYTIEAENQSGKKSATVLVKVYDTPGPCPSVKVKEVSRDSVTITWEIPTIDGGAPVNNYIVEKREAAMRAFKTVTTKCSKTLYRISGLVEGTMYYFRVLPENIYGIGEPCETSDAVLVSEVPLVPAKLEVVDVTKSTVTLAWEKPLYDGGSRLTGYVLEACKAGTERWMKVVTLKPTVLEHTVTSLNEGEQYLFRIRAQNEKGVSEPRETVTAVTVQDLRVLPTIDLSTMPQKTIHVPAGRPVELVIPIAGRPPPAASWFFAGSKLRESERVTVETHTKVAKLTIRETTIRDTGEYTLELKNVTGTTSETIKVIILDKPGPPTGPIKIDEIDATSITISWEPPELDGGAPLSGYVVEQRDAHRPGWLPVSESVTRSTFKFTRLTEGNEYVFRVAATNRFGIGSYLQSEVIECRSSIRIPGPPETLQIFDVSRDGMTLTWYPPEDDGGSQVTGYIVERKEVRADRWVRVNKVPVTMTRYRSTGLTEGLEYEHRVTAINARGSGKPSRPSKPIVAMDPIAPPGKPQNPRVTDTTRTSVSLAWSVPEDEGGSKVTGYLIEMQKVDQHEWTKCNTTPTKIREYTLTHLPQGAEYRFRVLACNAGGPGEPAEVPGTVKVTEMLEYPDYELDERYQEGIFVRQGGVIRLTIPIKGKPFPICKWTKEGQDISKRAMIATSETHTELVIKEADRGDSGTYDLVLENKCGKKAVYIKVRVIGSPNSPEGPLEYDDIQVRSVRVSWRPPADDGGADILGYILERREVPKAAWYTIDSRVRGTSLVVKGLKENVEYHFRVSAENQFGISKPLKSEEPVTPKTPLNPPEPPSNPPEVLDVTKSSVSLSWSRPKDDGGSRVTGYYIERKETSTDKWVRHNKTQITTTMYTVTGLVPDAEYQFRIIAQNDVGLSETSPASEPVVCKDPFDKPSQPGELEILSISKDSVTLQWEKPECDGGKEILGYWVEYRQSGDSAWKKSNKERIKDKQFTIGGLLEATEYEFRVFAENETGLSRPRRTAMSIKTKLTSGEAPGIRKEMKDVTTKLGEAAQLSCQIVGRPLPDIKWYRFGKELIQSRKYKMSSDGRTHTLTVMTEEQEDEGVYTCIATNEVGEVETSSKLLLQATPQFHPGYPLKEKYYGAVGSTLRLHVMYIGRPVPAMTWFHGQKLLQNSENITIENTEHYTHLVMKNVQRKTHAGKYKVQLSNVFGTVDAILDVEIQDKPDKPTGPIVIEALLKNSAVISWKPPADDGGSWITNYVVEKCEAKEGAEWQLVSSAISVTTCRIVNLTENAGYYFRVSAQNTFGISDPLEVSSVVIIKSPFEKPGAPGKPTITAVTKDSCVVAWKPPASDGGAKIRNYYLEKREKKQNKWISVTTEEIRETVFSVKNLIEGLEYEFRVKCENLGGESEWSEISEPITPKSDVPIQAPHFKEELRNLNVRYQSNATLVCKVTGHPKPIVKWYRQGKEIIADGLKYRIQEFKGGYHQLIIASVTDDDATVYQVRATNQGGSVSGTASLEVEVPAKIHLPKTLEGMGAVHALRGEVVSIKIPFSGKPDPVITWQKGQDLIDNNGHYQVIVTRSFTSLVFPNGVERKDAGFYVVCAKNRFGIDQKTVELDVADVPDPPRGVKVSDVSRDSVNLTWTEPASDGGSKITNYIVEKCATTAERWLRVGQARETRYTVINLFGKTSYQFRVIAENKFGLSKPSEPSEPTITKEDKTRAMNYDEEVDETREVSMTKASHSSTKELYEKYMIAEDLGRGEFGIVHRCVETSSKKTYMAKFVKVKGTDQVLVKKEISILNIARHRNILHLHESFESMEELVMIFEFISGLDIFERINTSAFELNEREIVSYVHQVCEALQFLHSHNIGHFDIRPENIIYQTRRSSTIKIIEFGQARQLKPGDNFRLLFTAPEYYAPEVHQHDVVSTATDMWSLGTLVYVLLSGINPFLAETNQQIIENIMNAEYTFDEEAFKEISIEAMDFVDRLLVKERKSRMTASEALQHPWLKQKIERVSTKVIRTLKHRRYYHTLIKKDLNMVVSAARISCGGAIRSQKGVSVAKVKVASIEIGPVSGQIMHAVGEEGGHVKYVCKIENYDQSTQVTWYFGVRQLENSEKYEITYEDGVAILYVKDITKLDDGTYRCKVVNDYGEDSSYAELFVKGVREVYDYYCRRTMKKIKRRTDTMRLLERPPEFTLPLYNKTAYVGENVRFGVTITVHPEPHVTWYKSGQKIKPGDNDKKYTFESDKGLYQLTINSVTTDDDAEYTVVARNKYGEDSCKAKLTVTLHPPPTDSTLRPMFKRLLANAECQEGQSVCFEIRVSGIPPPTLKWEKDGQPLSLGPNIEIIHEGLDYYALHIRDTLPEDTGYYRVTATNTAGSTSCQAHLQVERLRYKKQEFKSKEEHERHVQKQIDKTLRMAEILSGTESVPLTQVAKEALREAAVLYKPAVSTKTVKGEFRLEIEEKKEERKLRMPYDVPEPRKYKQTTIEEDQRIKQFVPMSDMKWYKKIRDQYEMPGKLDRVVQKRPKRIRLSRWEQFYVMPLPRITDQYRPKWRIPKLSQDDLEIVRPARRRTPSPDYDFYYRPRRRSLGDISDEELLLPIDDYLAMKRTEEERLRLEEELELGFSASPPSRSPPHFELSSLRYSSPQAHVKVEETRKDFRYSTYHIPTKAEASTSYAELRERHAQAAYRQPKQRQRIMAEREDEELLRPVTTTQHLSEYKSELDFMSKEEKSRKKSRRQREVTEITEIEEEYEISKHAQRESSSSASRLLRRRRSLSPTYIELMRPVSELIRSRPQPAEEYEDDTERRSPTPERTRPRSPSPVSSERSLSRFERSARFDIFSRYESMKAALKTQKTSERKYEVLSQQPFTLDHAPRITLRMRSHRVPCGQNTRFILNVQSKPTAEVKWYHNGVELQESSKIHYTNTSGVLTLEILDCHTDDSGTYRAVCTNYKGEASDYATLDVTGGDYTTYASQRRDEEVPRSVFPELTRTEAYAVSSFKKTSEMEASSSVREVKSQMTETRESLSSYEHSASAEMKSAALEEKSLEEKSTTRKIKTTLAARILTKPRSMTVYEGESARFSCDTDGEPVPTVTWLRKGQVLSTSARHQVTTTKYKSTFEISSVQASDEGNYSVVVENSEGKQEAEFTLTIQKARVTEKAVTSPPRVKSPEPRVKSPEAVKSPKRVKSPEPSHPKAVSPTETKPTPTEKVQHLPVSAPPKITQFLKAEASKEIAKLTCVVESSVLRAKEVTWYKDGKKLKENGHFQFHYSADGTYELKINNLTESDQGEYVCEISGEGGTSKTNLQFMGQAFKSIHEKVSKISETKKSDQKTTESTVTRKTEPKAPEPISSKPVIVTGLQDTTVSSDSVAKFAVKATGEPRPTAIWTKDGKAITQGGKYKLSEDKGGFFLEIHKTDTSDSGLYTCTVKNSAGSVSSSCKLTIKAIKDTEAQKVSTQKTSEITPQKKAVVQEEISQKALRSEEIKMSEAKSQEKLALKEEASKVLISEEVKKSAATSLEKSIVHEEITKTSQASEEVRTHAEIKAFSTQMSINEGQRLVLKANIAGATDVKWVLNGVELTNSEEYRYGVSGSDQTLTIKQASHRDEGILTCISKTKEGIVKCQYDLTLSKELSDAPAFISQPRSQNINEGQNVLFTCEISGEPSPEIEWFKNNLPISISSNVSISRSRNVYSLEIRNASVSDSGKYTIKAKNFRGQCSATASLMVLPLVEEPSREVVLRTSGDTSLQGSFSSQSVQMSASKQEASFSSFSSSSASSMTEMKFASMSAQSMSSMQESFVEMSSSSFMGISNMTQLESSTSKMLKAGIRGIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 3 - DNA, 19 bp, Artificial Sequence
  A synthetic sequence
agatttggacctgcgagcg

SEQ ID NO: 4 - DNA, 20 bp, Artificial Sequence
  A synthetic sequence
gagcggctgtctccacaagt

SEQ ID NO: 5 - DNA, 23 bp, Artificial Sequence
  A synthetic sequence
ttctgacctgaaggctctgcgcg

SEQ ID NO: 6 - DNA, 20 bp, Artificial Sequence
  A synthetic sequence
aaattttggggaccaggaac

SEQ ID NO: 7 - DNA, 20 bp, Artificial Sequence
  A synthetic sequence
tggcacctgtgtaggtcaac

SEQ ID NO: 8 - DNA, 20 bp, Artificial Sequence
  A synthetic sequence
atgtcgcgcattggcatgga

SEQ ID NO: 9 - DNA, 26 bp, Artificial Sequence
  A synthetic sequence
acaggtacgttaatagttaatagcgt

SEQ ID NO: 10 - DNA, 22 bp, Artificial Sequence
  A synthetic sequence
atattgcagcagtacgcacaca

SEQ ID NO: 11 - DNA, 26 bp, Artificial Sequence
  A synthetic sequence
acactagccatccttactgcgcttcg

SEQ ID NO: 12 - DNA, 19 bp, Artificial Sequence
  A synthetic sequence
agatttggacctgcgagcg

SEQ ID NO: 13 - DNA, 20 bp, Artificial Sequence
  A synthetic sequence
gagcggctgtctccacaagt

SEQ ID NO: 14 - DNA, 23 bp, Artificial Sequence
  A synthetic sequence
ttctgacctgaaggctctgcgcg

SEQ ID NO: 15 - DNA, 20 bp, Artificial Sequence
  A synthetic sequence
aaattttggggaccaggaac

SEQ ID NO: 16 - DNA, 20 bp, Artificial Sequence
  A synthetic sequence
tggcacctgtgtaggtcaac

SEQ ID NO: 17 - DNA, 20 bp, Artificial Sequence
  A synthetic sequence
atgtcgcgcattggcatgga